SEQUENCE LISTING (49 sequences)

SEQ ID NO: 1 - DNA, 354 bp, Artificial Sequence
  B7-VH Full Sequence
gaggtgcagctgttggagtctgggggaggcttggtacagcctggggggtccctgagactctcctgtgcagcctctggattcacctttagccactatgccatgaactgggtccgccaggctccagggaaggggctggagtgggtttcagccattaatagtaatggtgatagcacctactatccagacactgtgaaggaccgattcaccatctccagagacaattccaagaacacgctgtatctgcaaatgaacagcctgagagccgaggacacggccgtgtattactgtgcgagagatcggcgagcagggtactttgactactggggccagggcaccctggtcacagtctcctca

SEQ ID NO: 2 - protein, 118 aa, Artificial Sequence
  B7-VH  Full Sequence
EVQLLESGGGLVQPGGSLRLSCAASGFTFSHYAMNWVRQAPGKGLEWVSAINSNGDSTYYPDTVKDRFTISRDNSKNTLYLQMNSLRAEDTAVYYCARDRRAGYFDYWGQGTLVTVSS

SEQ ID NO: 3 - DNA, 75 bp, Artificial Sequence
  B7-VH FR1
gaggtgcagctgttggagtctgggggaggcttggtacagcctggggggtccctgagactctcctgtgcagcctct

SEQ ID NO: 4 - protein, 25 aa, Artificial Sequence
  B7-VH FR1
EVQLLESGGGLVQPGGSLRLSCAAS

SEQ ID NO: 5 - DNA, 24 bp, Artificial Sequence
  B7-VH CDRH1
ggattcacctttagccactatgcc

SEQ ID NO: 6 - protein, 8 aa, Artificial Sequence
  B7-VH CDRH1
GFTFSHYA

SEQ ID NO: 7 - DNA, 51 bp, Artificial Sequence
  B7-VH FR2
atgaactgggtccgccaggctccagggaaggggctggagtgggtttcagcc

SEQ ID NO: 8 - protein, 17 aa, Artificial Sequence
  B7-VH FR2
MNWVRQAPGKGLEWVSA

SEQ ID NO: 9 - DNA, 24 bp, Artificial Sequence
  B7-VH CDRH2
attaatagtaatggtgatagcacc

SEQ ID NO: 10 - protein, 8 aa, Artificial Sequence
  B7-VH CDRH2
INSNGDST

SEQ ID NO: 11 - DNA, 114 bp, Artificial Sequence
  B7-VH FR3
tactatccagacactgtgaaggaccgattcaccatctccagagacaattccaagaacacgctgtatctgcaaatgaacagcctgagagccgaggacacggccgtgtattactgt

SEQ ID NO: 12 - protein, 38 aa, Artificial Sequence
  B7-VH FR3
YYPDTVKDRFTISRDNSKNTLYLQMNSLRAEDTAVYYC

SEQ ID NO: 13 - DNA, 36 bp, Artificial Sequence
  B7-VH CDRH3
gcgagagatcggcgagcagggtactttgactactgg

SEQ ID NO: 14 - protein, 12 aa, Artificial Sequence
  B7-VH CDRH3
ARDRRAGYFDYW

SEQ ID NO: 15 - DNA, 30 bp, Artificial Sequence
  B7-VH J Region
ggccagggcaccctggtcacagtctcctca

SEQ ID NO: 16 - protein, 10 aa, Artificial Sequence
  B7-VH J Region
GQGTLVTVSS

SEQ ID NO: 17 - DNA, 330 bp, Artificial Sequence
  B7- VL (lambda) Full Sequence
gatatcgtgctgactcagccaccctcagcgtctgggacccccgggcagagggtcaccatctcctgctctggaagcagggacaacatcgggaagaattatgtgtcctggtaccagcagcttccaggaacagcccccaaactcctcatttataggaataatcagcggccctcaggggtccctgaccggttctctggctccaagtctggcacctcagcctccctggccatcagtgggctgcggtccgaggatgaggctgattattactgctcagcgtgggacaccagcctcaatgcttatgtcttcgggcctggcacccagctcaccgtcctc

SEQ ID NO: 18 - protein, 110 aa, Artificial Sequence
  B7- VL (lambda) Full Sequence
DIVLTQPPSASGTPGQRVTISCSGSRDNIGKNYVSWYQQLPGTAPKLLIYRNNQRPSGVPDRFSGSKSGTSASLAISGLRSEDEADYYCSAWDTSLNAYVFGPGTQLTVL

SEQ ID NO: 19 - DNA, 75 bp, Artificial Sequence
  B7- VL FR1
gatatcgtgctgactcagccaccctcagcgtctgggacccccgggcagagggtcaccatctcctgctctggaagc

SEQ ID NO: 20 - protein, 25 aa, Artificial Sequence
  B7- VL FR1
DIVLTQPPSASGTPGQRVTISCSGS

SEQ ID NO: 21 - DNA, 24 bp, Artificial Sequence
  B7- VL CDRL1
agggacaacatcgggaagaattat

SEQ ID NO: 22 - protein, 8 aa, Artificial Sequence
  B7- VL CDRL1
RDNIGKNY

SEQ ID NO: 23 - DNA, 51 bp, Artificial Sequence
  B7- VL FR2
gtgtcctggtaccagcagcttccaggaacagcccccaaactcctcatttat

SEQ ID NO: 24 - protein, 17 aa, Artificial Sequence
  B7- VL FR2
VSWYQQLPGTAPKLLIY

SEQ ID NO: 25 - DNA, 10 bp, Artificial Sequence
  B7- VL CDRL2
  misc_feature (10)..(10): n is null
aggaataatn

SEQ ID NO: 26 - protein, 4 aa, Artificial Sequence
  B7- VL CDRL2
  MISC_FEATURE (4)..(4): Xaa is null
RNNX

SEQ ID NO: 27 - DNA, 108 bp, Artificial Sequence
  B7- VL FR3
cagcggccctcaggggtccctgaccggttctctggctccaagtctggcacctcagcctccctggccatcagtgggctgcggtccgaggatgaggctgattattactgc

SEQ ID NO: 28 - protein, 36 aa, Artificial Sequence
  B7- VL FR3
QRPSGVPDRFSGSKSGTSASLAISGLRSEDEADYYC

SEQ ID NO: 29 - DNA, 27 bp, Artificial Sequence
  B7- VL CDRL3
tcagcgtgggacaccagcctcaatgct

SEQ ID NO: 30 - protein, 9 aa, Artificial Sequence
  B7- VL CDRL3
SAWDTSLNA

SEQ ID NO: 31 - DNA, 36 bp, Artificial Sequence
  B7- VL J Region
tatgtcttcgggcctggcacccagctcaccgtcctc

SEQ ID NO: 32 - protein, 12 aa, Artificial Sequence
  B7- VL J Region
YVFGPGTQLTVL

SEQ ID NO: 33 - protein, 17 aa, Artificial Sequence
  PEPTIDE #49
CTSAQKASQVAEEAADC

SEQ ID NO: 34 - protein, 17 aa, Artificial Sequence
  PEPTIDE #49-SC
CSSEVQAAKADTEAQAC

SEQ ID NO: 35 - protein, 17 aa, Artificial Sequence
  PEPTIDE #50
CKASQVAEEAADAAQEC

SEQ ID NO: 36 - protein, 17 aa, Artificial Sequence
  PEPTIDE #50-SC
CDEAVAQAAAEKEAQSC

SEQ ID NO: 37 - protein, 21 aa, Artificial Sequence
  PEPTIDE #49+50
CTSAQKASQVAEEAADAAQEC

SEQ ID NO: 38 - protein, 17 aa, Artificial Sequence
  PEPTIDE #78
CTDLINRMGQAARLAGC

SEQ ID NO: 39 - protein, 18 aa, Artificial Sequence
  EPITOPE OF B7
TSAQKASQVAEEAADAAQ

SEQ ID NO: 40 - protein, 321 aa, Escherichia coli
  MISC_FEATURE: EspB, EPEC Accession number WP_001091991.1
MNTIDNNNAAIAVNSVLSSTTDSTSSTTTSTSSISSSLLTDGRVDISKLLLEVQKLLREMVTTLQDYLQKQLAQSYDIQKAVFESQNKAIDEKKAGATAALIGGAISSVLGILGSFAAINSATKGASDVAQQAASTSAKSIGTVSEASTKALAKASEGIADAADDAAGAMQQTIATAAKAASRTSGITDDVATSAQKASQVAEEAADAAQELAQKAGLLSRFTAAAGRISGSTPFIVVTSLAEGTKTLPTTISESVKSNHDINEQRAKSVENLQASNLDTYKQDVRRAQDDISSRLRDMTTTARDLTDLINRMGQAARLAG

SEQ ID NO: 41 - DNA, 945 bp, Escherichia coli
  misc_feature: EspB, EPEC Accession number AAB69980.1
atgaatactattgattatactaatcaagtaatgacggttaattctgtttcggagaatactaccggctctaatgcaattaccgcatctgctattaattcatctttgcttaccgatggtaaggtcgatgtttctaaactgatgctggaaattcaaaaactcctgggcaagatggtgcgtatattgcaggattaccaacagcaacagttgtcgcagagctatcagatccaactggccgtttttgagagccagaataaagccattgatgaaaaaaaggccgctgcaacagccgctctggttggtggggctatttcatcagtattggggatcttaggctcttttgcagcaattaacagtgctacgaaaggcgcgagtgatattgctcaaaaaaccgcctctacatcttctaaggctattgatgcggcttctgatactgcgactaaaacgttgactaaggcaacggaaagcgttgctgatgctgttgaagatgcatccagcgtgatgcagcaagcgatgactacagcaacgagagcggccagccgtacatccgacgttgctgatgacattgccgattctgctcagagagcttctcagctggctgaaaacgctgcagatgccgctcagaaggcaagtcgggcaagccgctttatggctgcagtagataagattactggctctacaccatttattgccgttaccagtcttgccgaaggcacgaagacattgccaacaacggtatctgaatcagtcaaatctaaccatgagattagcgaacagcgttataagtctgtggagaacttccagcagggtaatttggatctgtataagcaagaagttcgcagagcgcaggatgatatcgctagccgtctgcgtgatatgacaacagccgctcgcgatctcactgatcttcagaatcgtatgggtcaatcggttcgcttagctgggtaa

SEQ ID NO: 42 - protein, 380 aa, Escherichia coli
  MISC_FEATURE: EspD, EPEC Accession number WP_000935767.1
MLNVNSDIQSMRSGASAATATSGINQPEVTSALDLQLVKSTAPSASWTESTALPTPPAGHSLVTPSAAEDVLSKLFGGISGEVRSRTEGTEPQRSTQNASSGYPYLSQVNNVDPQAMMMMVTLLSLDASAQKVASMKNSNEIYTDGQNKALDNKTLEFKKQLEEQQKAEEKAQKSKIVGQVFGWLGVAVTAIAAIFNPALWAVVAVSATAMALQTAVDVMGDKAPQALKTAAQVFGGVSIAASILTAGIGGVSSLMSKVGDVANKVGSNIVKTVTKLADVFVENVVSKVAATANGFTTSARSIGTTVLNNDSAQYSILAQLSAYAVQNLTRQSENLGESAKVELDKAAAELRNQASYLQNASQLISDSARVNSRIVSGRV

SEQ ID NO: 43 - DNA, 1000 bp, Escherichia coli
  misc_feature: EspD, EPEC Accession number CAI43861.1
atgctcaatgtaaatagcgatatccagtcgatgaggtctggggccagcgctgctacggctacttcaggtattaatcagcctgaagtgacttccgcgctggatttacaactggttaaatccacggccccatcagcaagttggacagaaagtaccgctttaccaacgccaccggcaggccactcattagtgacgccctctgctgctgaggatgtcctgagtaaactgtttggtggtattagtggtgaagttaggagtcgaacagagggaaccgaaccacagcgcagcactcagaatgcaagctctggctatccttatctttctcaggtgaataacgttgaccctcaggccatgatgatgatggtcaccctgttatccctggatgcttcagcgcagaaagttgcgagtatgaaaaactccaacgagatctacacggatgggcaaaataaggcgctggataataaaacgctggagtttaaaaagcagctcgaagaacaacagaaagccgaagagaaagcacaaaaaagtaaaattgttggtcaggtctttggttggttgggcgtcgcggtaactgctattgcagctatttttaacccggcactctgggctgttgttgccgttagcgcaacagcaatggcactgcaaactgcagttgatgtaatgggcgataaggccccgcaggcgttaaagacagcagcgcaagtctttggcggagtatctattgctgcaagtattctgacagccggcattggcggggtgtcttcacttatgtctaaagttggcgatgtcgctaacaaagttggttcaaacatcgtgaaaacagttactaaactggccgatgtatttgttgagaacgttgtttcaaaagttgcggcaacggcaaatggatttacgacctctgcacgttctattgggacaactgtgctgaataatgattctgcgcaatacagcatattggctcagttatctgcttacgcggtgcaaaacttaactcgacagagtg

SEQ ID NO: 44 - protein, 21 aa, Artificial Sequence
  PEPTIDE 49-50 SC
CAEATQEADVAEQASKASQAC

SEQ ID NO: 45 - protein, 312 aa, Escherichia coli
  MISC_FEATURE: EHEC-EspB
MNTIDNTQVTMVNSASESTTGASSAVAASALSIDSSLLTDGKVDICKLMLEIQKLLGKMVTLLQDYQQKQLAQSYQIQQAVFESQNKAIEEKKAAATAALVGGIISSALGILGSFAAMNNAAKGAGEIAEKASSASSKAAGAASEVANKALVKATESVADVAEEASSAMQKAMATTTKAASRASGVADDVAKASDFAEDLADAAEKTSRINKLLNSVDKLTNTTAFVAVTSLAEGTKTLPTTISESVKSTHEVNEQRAKSLENFQQGNLELYKQDVRRTQDDITTRLRDITSAVRDLLEVQNRMGQSGRLAG

SEQ ID NO: 46 - protein, 321 aa, Citrobacter rodentium
  MISC_FEATURE: C. rodentium (CR)-EspB
MNTIDYNNAAIAVNPVVSSTTDSASSTTTLTSSSSSSLLTAGKVDISKLLLEVQKLMREMVTTLQDYLQRQLAQSYNIQQAIFQSQNKAIEEKKAGATAALIGGAISSVLGILGSFAAINSATKGASDVVQKATSASAKSVNTASEVSTKALVKASESIADAADETVGTLQQGIAAATKAANRTSGIADDVTTSAQKASQVAEEAADAAQDLTRKANLLSRFTAAAGRISGSTAFVVVTSLAEGTKTLPTTISESVKSNHDINEQRAKSVENLQASNLETYKQDVRRSQDDIASRLRDMTATARDLNDLINRMGQSARLAG

SEQ ID NO: 47 - protein, 409 aa, Artificial Sequence
  Salmonella-EspB A
MLISNVGINPAAYLNNHSVENSSQTASQSVSAKDILNSIGISSSKVSDLGLSPTLSAPAPGVLTQTPGTITSFLKASIQNTDMNQDLNALANNVTTKANEVVQTQLREQQAEVGKFFDISGMSSSAVALLAAANTLMLTLNQADSKLSGKLSLVSFDAAKTTASSMMREGMNALSGSISQSALQLGITGVGAKLEYKGLQNERGALKHNAAKIDKLTTESHSIKNVLNGQNSVKLGAEGVDSLKSLNMKKTGTDATKNLNDATLKSNAGTSATESLGIKDSNKQISPEHQAILSKRLESVESDIRLEQNTMDMTRIDARKMQMTGDLIMKNSVTVGGIAGASGQYAATQERSEQQISQVNNRVASTASDEARESSRKSTSLIQEMLKTMESINQSKASALAAIAGNIRA

SEQ ID NO: 48 - protein, 27 aa, Artificial Sequence
  peptide #9-12
SISSSLLTDGRVDISKLLLEVQKLLRE

SEQ ID NO: 49 - protein, 11 aa, Artificial Sequence
  Epitope peptide
KASQVAEEAAD

(19) United States
(12) Patent Application Publication
(10) Pub. No.: US 2025/0277767 A1
(43) Pub. Date: Sep. 4, 2025

(54) MONOCLONAL ANTIBODY-BASED BIOSENSOR FOR POINT-OF-CARE DETECTION OF TYPE III SECRETION SYSTEM EXPRESSING PATHOGENS

(71) Applicants: RAMOT AT TEL-AVIV UNIVERSITY LTD., Tel Aviv (IL); B.G. NEGEV TECHNOLOGIES AND APPLICATIONS LTD., AT BEN-GURION UNIVERSITY, Beer Sheva (IL); THE STATE OF ISRAEL, MINISTRY OF AGRICULTURE & RURAL DEVELOPMENT, AGRICULTURAL RESEARCH ORGANIZATION, Rishon Lezion (IL)

(72) Inventors: Yariv WINE, Tel Aviv (IL); Yaron HILLMAN, Tel Aviv (IL); Dan EVEN, Tel Aviv (IL); Dan LUSTIGER, Tel Aviv (IL); Neta SAL-MAN, Beer Sheva (IL); Dor BRAVERMAN, Beer Sheva (IL); Jenia GERSHBERG, Beer Sheva (IL); Sefi VERNICK, Rishon Lezion (IL); Idan ASHUR, Rishon Lezion (IL)

(21) Appl. No.: 18/254,489

(22) PCT Filed: Nov. 15, 2021
(86) PCT No.: PCT/IL2021/051406

§ 371 Date: May 25, 2023

Related U.S. Application Data
(60) Provisional application No. 63/118,371, filed on Nov. 25, 2020.

(51) Int. Cl. G01N 27/327; G01N 27/02; G01N 33/543; G01N 33/569; G01N 33/68

(52) U.S. Cl. CPC G01N 27/3276; G01N 27/026; G01N 33/5438; G01N 33/56911; G01N 33/6893; G01N 2333/245

(57) ABSTRACT

The present disclosure relates to a biosensor chip device usable for identifying and/or quantifying a target in a sample by electrochemical impedance spectroscopy (EIS) analysis. The biosensor chip device disclosed herein comprises a plurality of electrodes connectable to at least one electronic device, wherein, at least one of the electrodes is a working electrode connected directly or indirectly to at least one target binding site and/or moiety. It should be noted that the target binding site and/or moiety specifically binds the at least one target or any component thereof. Still further, in some embodiments, the plurality of electrodes is configured for electrochemical impedance spectroscopy (EIS) analysis of the sample. More specifically, the invention relates to monoclonal-antibody-based biosensor chip, devices, kits and diagnostic methods for detection of pathogens expressing Type III secretion system (T3SS) in a sample.

The Sequence Listing in ASCII text file format of 31,649 bytes in size, created on Jan. 25, 2024, with the file name “2024-01-26SequenceListing_WINE1A,” filed in the U.S. Patent and Trademark Office on even date herewith, is hereby incorporated herein by reference.

### FIELD OF THE INVENTION

The present invention generally relates to diagnostic devices, kits, methods and uses thereof in detection of pathogens. More specifically, the invention relates to monoclonal-antibody-based biosensor chip, devices, kits and diagnostic methods for detection of pathogens expressing Type III secretion system (T3SS).

### BACKGROUND ART

References considered to be relevant as background to the presently disclosed subject matter are listed below:
1. Croxen, M. A.; Law, R. J.; Scholz, R.; Keeney, K. M.; Wlodarska, M.; Finlay, B. B., Recent advances in understanding enteric pathogenic Escherichia coli. Clin Microbiol Rev 2013, 26 (4), 822-80.
2. Guttikonda, S.; Tang, X. L.; Yang, B. M.; Armstrong, G. D.; Suresh, M. R., Monospecific and bispecific antibodies against E. coli 0157 for diagnostics. J Immunol Methods 2007, 327 (1-2), 1-9.
3. Jampasa, S.; Lae-Ngee, P.; Patarakul, K.; Ngamrojanavanich, N.; Chailapakul, O.; Rodthongkum, N., Electrochemical immunosensor based on gold-labeled monoclonal anti-LipL32 for leptospirosis diagnosis. Biosens Bioelectron 2019, 142, 111539.
4. Kaper, J. B.; Nataro, J. P.; Mobley, H. L., Pathogenic Escherichia coli. Nature reviews. Microbiology 2004, 2 (2), 123-40.
5. Cornelis, G. R., The type III secretion injectisome. Nature reviews. Microbiology 2006, 4 (11), 811-25.
6. Mellies, J. L.; Barron, A. M.; Carmona, A. M., Enteropathogenic and enterohemorrhagic Escherichia coli virulence gene regulation. Infect Immun 2007, 75 (9), 4199-210.
7. Daniell, S. J.; Takahashi, N.; Wilson, R.; Friedberg, D.; Rosenshine, I.; Booy, F. P.; Shaw, R. K.; Knutton, S.; Frankel, G.; Aizawa, S., The filamentous type III secretion translocon of enteropathogenic Escherichia coli. Cell Microbiol 2001, 3 (12), 865-71.
8. Luo, W.; Donnenberg, M. S., Analysis of the function of enteropathogenic Escherichia coli EspB by random mutagenesis. Infect Immun 2006, 74 (2), 810-20.
9. Azriel-Rosenfeld, R.; Valensi, M.; Benhar, I., A human synthetic combinatorial library of arrayable single-chain antibodies based on shuffling in vivo formed CDRs into general framework regions. Journal of molecular biology 2004, 335 (1), 177-92.
10. Vaisman-Mentesh, A.; Rosenstein, S.; Yavzori, M.; Dror, Y.; Fudim, E.; Ungar, B.; Kopylov, U.; Picard, O.; Kigel, A.; Ben-Horin, S.; Benhar, I.; Wine, Y., Molecular Landscape of Anti-Drug Antibodies Reveals the Mechanism of the Immune Response Following Treatment With TNFalpha Antagonists. Front Immunol 2019, 10, 2921.
11. Porat-Ophir, C.; Belkin, A.; Vernick, S.; Dergachev, V.; Freynd, G.; Katsnelson, M.; Shacham-Diamand, Y., Electrochemical Biochip Characterization of the Effect of Formaldehyde on the Activity of Alkaline Phosphatase. Ecs Electrochem Lett 2013, 2 (12), G8-G10.
12. Vernick, S.; Niv, Y.; Vilkin, A.; Freeman, A.; Shacham-Diamand, Y., Colon Cancer Diagnosis by Multiple Biomarker Electrobiochemical Detection in Biopsy Slices. Gastroenterology 2012, 142 (5), S345-S345.
13. Vernick, S.; Freeman, A.; Rishpon, J.; Niv, Y.; Vilkin, A.; Shacham-Diamand, Y., Electrochemical Biosensing for Direct Biopsy Slices Screening for Colorectal Cancer Detection. Journal of The Electrochemical Society 2011, 158 (1), P1-P4.
14. Luo, W.; Donnenberg, M. S., Interactions and predicted host membrane topology of the enteropathogenic Escherichia coli translocator protein EspB. J Bacteriol 2011, 193 (12), 2972-80.
15. Randviir, E. P.; Banks, C. E., Electrochemical impedance spectroscopy: an overview of bioanalytical applications. Analytical Methods 2013, 5 (5), 1098-1115.
16. Siddiqui, S.; Dai, Z.; Stavis, C. J.; Zeng, H.; Moldovan, N.; Hamers, R. J.; Carlisle, J. A.; Arumugam, P. U., A quantitative study of detection mechanism of a label-free impedance biosensor using ultrananocrystalline diamond microelectrode array. Biosens Bioelectron 2012, 35 (1), 284-290.
17. Lasia, A., Impedance of the Faradaic Reactions in the Presence of Mass Transfer. In Electrochemical Impedance Spectroscopy and its Applications, Springer New York: New York, NY, 2014; pp 85-125.
18. Hearty, S.; Leonard, P.; Quinn, J.; O'Kennedy, R., Production, characterisation and potential application of a novel monoclonal antibody for rapid identification of virulent Listeria monocytogenes. J Microbiol Methods 2006, 66 (2), 294-312.
19. Barreiros dos Santos, M.; Agusil, J. P.; Prieto-Simon, B.; Sporer, C.; Teixeira, V.; Samitier, J., Highly sensitive detection of pathogen Escherichia coli O157:H7 by electrochemical impedance spectroscopy. Biosens Bioelectron 2013, 45, 174-80.
20. Joung, C. K.; Kim, H. N.; Lim, M. C.; Jeon, T. J.; Kim, H. Y.; Kim, Y. R., A nanoporous membrane-based impedimetric immunosensor for label-free detection of pathogenic bacteria in whole milk. Biosens Bioelectron 2013, 44, 210-5.
21. Lu, L.; Chee, G.; Yamada, K.; Jun, S., Electrochemical impedance spectroscopic technique with a functionalized microwire sensor for rapid detection of foodborne pathogens. Biosens Bioelectron 2013, 42, 492-5.
22. Barreiros dos Santos, M.; Sporer, C.; Sanvicens, N.; Pascual, N.; Errachid, A.; Martinez, E.; Marco, M. P.; Teixeira, V.; Samiter, J., Detection of pathogenic Bacteria by Electrochemical Impedance Spectroscopy: Influence of the immobilization strategies on the sensor performance. Procedia Chemistry 2009, 1 (1), 1291-1294.

### BACKGROUND OF THE INVENTION

The emergence of multiple-drug resistant (MDR) bacterial strains stems largely from the extensive, and sometimes inappropriate, usage of antibiotics in the community and in agriculture, as this misuse has exerted a strong selective pressure on bacteria to develop resistance mechanisms against various antibiotics. In turn, the implications of the increasing numbers of MDR bacterial infections in the clinic, in the community, and in agriculture are constituting a growing global public health concern. MDR bacterial infections are harder to treat and are associated with higher medical costs than antibiotic-sensitive infections, and, perhaps more importantly, there is a significant risk that MDR mechanisms will be spread to other bacterial strains. A parallel public health concern is that the development and approval of new antibiotics has not kept pace with the rising rates of morbidity and mortality due to bacterial infections, giving rise to a predicted annual death rate of 10 million people by 2050 due to resistance to antimicrobials. The lack of progress in the development of antibiotics may be attributed not only to the limited discovery of suitable molecular targets, but also to the absence of significant investment of large pharmaceutical companies.

Pivotal to the efficiency of controlling antibiotic resistance is the ability to provide rapid and accurate surveillance and diagnosis, as is embodied in the WHO One Health concept for addressing the MDR crisis. In this regard, the major disadvantages of currently available laboratory-based diagnostics for the detection of bacterial infections are long processing times, low sensitivity and specificity, and/or the need for specialized equipment that is expensive and requires highly trained personnel. Among the laboratory-based methods currently in use for bacterial diagnosis, bacterial culturing is probably the most frequently used method, but it is relatively slow and it is limited to bacteria that can be cultured in the laboratory. Other methods are based on immunoassays [including enzyme-linked immunosorbent assays (ELISA) and agglutination assays] that detect surface bacterial antigens and on genetic analyses that allow rapid identification of bacterial strains by employing a polymerase chain reaction (PCR). The latter methods are the most sensitive, but even they may yield false-positive results and they may overlook genetically mutated strains. A possible solution was thought to lie in rapid real-time PCR or mass-spectroscopy techniques, but these, too, require specialized equipment and reagents and trained personnel [1]. The above-described obstacles may culminate in misdiagnosed or belatedly diagnosed bacterial infections and the misuse of antibiotics, and hence, ultimately, in the exacerbation of the antibiotic resistance crisis.

A particularly promising means for providing such diagnosis lies in monoclonal antibodies (mAbs) targeted against pathogen-specific antigens. mAbs were previously demonstrated as diagnostic agents for the detection of harmful bacteria [2, 3]. In keeping with this line of thought, recent advances in the discovery, engineering, production, and clinical development of mAbs indicate their potential in the design of rapid diagnostics.

A major need for rapid diagnosis includes strains of Gram-negative bacterial pathogens, such as Escherichia coli, and species of Salmonella, Shigella, Yersinia, and Pseudomonas, which cause serious diseases, ranging from lethal diarrhea to sepsis, leading to millions of deaths annually [1]. An essential component common to these bacterial pathogens is termed the type 3 secretion system (T3SS). The T3SS is a syringe-like protein complex, which is responsible for injecting virulence factors from the bacterial cytoplasm directly into the human host cell [4]. This T3SS complex is essential for bacterial virulence, as the injected proteins (effectors) manipulate key intracellular host pathways (e.g., cell cycle, immune response, cytoskeletal organization, metabolic processes and intracellular trafficking) that ultimately promote bacterial replication and transmission [5].

The present disclosure focused on the development of a T3SS-specific mAb and its use in a bioelectronic diagnostic device for the detection of enteropathogenic E. coli (EPEC), that according to [1], is the causative agent of infantile diarrhea. EPEC contains a T3SS, which is absent from the non-pathogenic strains of E. coli. The EPEC T3SS comprises more than 20 proteins, three of which, EspA, EspB, and EspD, are highly exposed to the extracellular environment. EspA forms a long filamentous structure that bridges between the bacterial and host cells, and EspB and EspD together form a translocator pore complex that facilitates the passage of effectors across the host plasma membrane.

Sensitive devices and systems based on antibodies specific for T3SS components, specifically, antibodies directed at EspB, may provide a powerful and rapid point-of-care diagnostic solution for monitoring bacterial infections in the community. There is, thus, an imperative need for more rapid, cost-effective, and sensitive assays that can identify infective agents at the point of care (POC), without the requirement for multistep processing.

### SUMMARY OF THE INVENTION

A first aspect of the present disclosure relates to a biosensor chip device usable for identifying and/or quantifying at least one target in a sample by electrochemical impedance spectroscopy (EIS) analysis, the chip device comprises a plurality of electrodes connectable to at least one electronic device. In some embodiments, at least one of the electrodes is a working electrode. Such working electrode is connected, directly or indirectly, to at least one target binding site/moiety. In yet some further embodiments, the target binding site and/or moiety, specifically binds the at least one target or any component thereof. Still further, it should be noted that in some embodiments, the plurality of electrodes is configured for electrochemical impedance spectroscopy (EIS) analysis of the sample.

In yet some further embodiments, the chip device of the present disclosure comprises an electrode arrangement having electric contacts connectable to an electronic device configured for EIS, and active ends positioned within a measurement chamber. Still further, in some embodiments, the electrode arrangement comprises two or more electrodes, wherein at least one of the electrodes (referred to herein as the working electrode) carries at least one target binding site and/or moiety for binding one or more targets or any components thereof, in a sample to be tested.

### In Some Embodiments, the Chip Device Comprises:

(a) a substrate portion having a plurality of electrodes formed in an electrodes portion thereof, and at least one electronic circuitry (e.g., potentiostat circuitry) electrically connected to the electrodes; wherein at least one of the electrodes is connected directly or indirectly to at least one target binding site and/or moiety; and (b) a packaging assembly configured to sealably enclose the electrodes portion of the substrate and define a measurement chamber encompassing said electrodes.

### A Further Aspect of the Present Disclosure Relates to a Kit Comprising:

First (a), at least one biosensor chip device usable for identifying and/or quantifying a target in a sample by electrochemical impedance spectroscopy (EIS) analysis, the chip device comprising: a plurality of electrodes connectable to at least one electronic device. In some embodiments, at least one of the electrodes is a working electrode. More specifically, the working electrode is connected either directly or indirectly to at least one target binding site and/or moiety. It should be noted that the target binding site and/or moiety specifically targets and binds the at least one target or any component thereof. Still further, in some embodiments, the plurality of electrodes is configured for electrochemical impedance spectroscopy (EIS) analysis of the sample.

In some embodiments, the at least one biosensor chip device of the disclosed kit may further comprise a substrate portion having a plurality of electrodes formed in an electrodes portion thereof. According to these embodiments, the at least one electronic device of the disclosed biosensor device comprises a plurality of potentiostat circuitries. Accordingly, in some embodiments the biosensor chip device of the present disclosure comprises at least one potentiostat circuitry electrically connected to the electrodes. It should be noted that at least one of the electrodes is connected directly or indirectly to at least one target binding site and/or moiety. Still further, in some embodiments, the biosensor chip device of the disclosed kit may further comprise a packaging assembly configured to sealably enclose the electrodes portion of the substrate and define a measurement chamber encompassing the electrodes.

In some embodiments, the kit of the present disclosure optionally further comprises at least one of: (b) at least one control sample and/or control standard value, and (c) instructions for use. Another aspect of the present disclosure relates to a method for identifying and/or quantifying at least one target in a sample. More specifically, the method comprising:
contacting a plurality of electrodes comprising at least one working electrode and at least one reference electrode with the sample. Still further, in some embodiments, the plurality of electrodes used by the disclosed methods also comprises at least one counter electrode. It should be noted that at least one working electrode is connected directly or indirectly to at least one target binding site and/or moiety. The next step involves applying voltage signal between the at least one working electrode and the at least one counter electrode and determining electrical current between the electrodes in response to the voltage signals for a selected number of one or more signal frequencies. The applied voltage may be determined based on electrical voltage between the working electrode and reference electrode. The next step involves determining relations between electrical current response and voltage signal for the one or more signal frequencies; and determining electrical impedance between the at least one working electrode and the at least one counter electrode. It should be noted that the impedance variation being indicative of presence and/or quantity of the at least one target in the sample.

A further aspect of the present disclosure relates to a method for identifying and/or quantifying at least one target in a sample. More specifically, the method comprising the following steps: The first step involves contacting at least one sample with a plurality of electrodes comprising at least one working electrode and at least one reference electrode. Still further, in some embodiments, the plurality of electrodes used by the disclosed methods also comprises at least one counter electrode. It should be noted that the at least one working electrode is connected directly or indirectly to at least one target binding site and/or moiety.

The next step involves measuring electrical voltages between the at least one working electrode and said at least one reference electrode in response to electric currents of different frequencies applied by the at least one counter electrode.

The next step involves determining electrical impedances based on the measured electrical voltage and the electric currents applied at the different frequencies.

In the next step, determining a charge transfer electrical resistance based on the determined impedances is performed.

The following step involves determining presence of the target in the sample whenever the charge transfer electrical resistance determined in the previous step is greater than a predetermined threshold value.

A further aspect of the present disclosure relates to a method of treating, preventing, ameliorating, reducing or delaying the onset of an infection by at least one bacteria expressing at least one T3SS in a subject in need thereof. In more specific embodiments, the method comprising:

In step (a), classifying a subject as infected by said bacteria if the presence of at least one T3SS component is determined in at least one sample of the subject. In some embodiments, determination of the presence of the at least one T3SS component in the sample comprises the step of:
contacting the at least one sample of the subject with a plurality of electrodes comprising at least one working electrode and at least one reference electrode, or any biosensor chip or kit comprising the electrodes. Still further, in some embodiments, the plurality of electrodes may further comprise at least one counter electrode. In some embodiments, at least one working electrode is connected directly or indirectly to at least one target binding site and/or moiety;
measuring electrical voltages between the at least one working electrode and the at least one reference electrode in response to electric currents of different frequencies applied by the at least one counter electrode;
determining electrical impedances based on the measured electrical voltage and the electric currents applied at the different frequencies;
determining a charge transfer electrical resistance based on the determined impedances; and determining presence of the bacteria expressing at least one T3SS in said sample whenever said charge transfer electrical resistance is greater than a predetermined threshold value, thereby classifying said subject as infected by the bacteria.

The next step (b), involves administering to a subject classified in step (a), as an infected subject, a therapeutically effective amount of at least one anti-bacterial agent.

Still further, in an additional aspects thereof, the present disclosure provides t least one system comprising any of the biosensor chip devices disclosed herein.

These and other aspects of the invention will become apparent by the hand of the following description.

### BRIEF DESCRIPTION OF THE DRAWINGS

In order to better understand the subject matter that is disclosed herein and to exemplify how it may be carried out in practice, embodiments will now be described, by way of non-limiting example only, with reference to the accompanying drawings, in which:

FIG. 1A-1B. mAb-EspB-B7 Binds EspB with High Affinity

FIG. 1A. mAb-EspB-B7 binding affinity to purified EspB was evaluated by ELISA. A 96-well plate coated with EspB was incubated with serially diluted mAb-EspB-B7. mAb-EspB-B7 binding was determined using anti-human IgG HRP-conjugated antibody. Error bars represent ±SD.

FIG. 1B. SPR sensorgrams of mAb-EspB-B7 binding to an EspB-coated chip. mAb-EspB-B7 was added at various concentrations between 10 and 90 nM. Sensorgrams were fitted to the steady-state model.

FIG. 2A-2B. mAb-EspB-B7 Binds to Recombinant and Native EspB

FIG. 2A. EPEC wild type (WT), ΔescN, ΔespB and ΔespB expressing EspB-His strains were grown under T3SS-inducing conditions for 6 hr. The bacterial pellets and supernatants were separated and analyzed using SDS-PAGE and western blotting with mAb-EspB-B7. EspB expression within the bacteria (pellet) was observed only for the ΔespB+EspB-His strain, while EspB secretion (supernatant) was observed for both WT EPEC and the complemented ΔespB+EspB-His strain.

FIG. 2B. EPEC WT, ΔescN, ΔespB and ΔespB+EspB-His bacteria were grown under T3SS-inducing conditions for 3 hr. Thereafter, 1×10⁷ bacteria were incubated with mAb-EspB-B7, washed, and stained with Alexa Fluor 488 goat anti-human IgG antibody. Flow cytometry analysis was performed on a Gallios instrument (Beckman coulter).

FIG. 3A-3C. mAb-EspB-B7 Binding to EspB Under Various Conditions

mAb-EspB-B7 binding to EspB was evaluated by ELISA.

FIG. 3A. shows evaluation of the binding in different media.

FIG. 3B. shows evaluation of the binding under various pH conditions.

FIG. 3C. shows evaluation of the binding at different NaCl concentrations. Error bars represent ±SD.

FIG. 4. mAb-EspB-B7 is thermally stable

The melting temperatures (Tₘ) of mAb-EspB-B7 alone or in combination with recombinant EspB were determined by nano Differential Scanning Fluorimetry (nanoDSF), Prometheus NT.48, NanoTemper.

FIG. 5. mAb-EspB-B7 does not Interfere with the EspB-EspD Interaction

Supernatants of EPEC ΔespD expressing EspD-³⁵His were purified using Ni-NTA beads. EPEC ΔespD strain without the pEspD-³⁵His expression vector, was used as a negative control. Samples of supernatants (S) and elution (E) fractions were loaded on SDS-PAGE and analyzed by western blotting with mouse anti-His and anti-EspB antibodies (to avoid detection of the human EspB antibody). Analysis of the supernatants confirmed EspB and EspD secretion into the extracellular medium. The co-elution of EspB with EspD-³⁵His was not affected by the absence or presence (100 nM and 200 nM) of mAb-EspB-B7. Low EspB non-specific binding to the Ni-NTA beads was detected (in the absence of EspD-³⁵His).

FIG. 6A-6D. mAb-EspB-B7 Epitope Mapping

FIG. 6A. An EspB pepstar peptide array of 78 cyclic peptides (15-residue long peptides with an 11-residue overlap) was examined for mAb-EspB-B7 binding. Image analysis was carried out with Genepix Pro 6.0 analysis software (Molecular Devices) to detect antibody binding; fluorescence signals were normalized showing their relative intensities. The putative binding site of mAb-EspB-B7 along the EspB protein is marked in light gray. Arrows indicate the signals obtained from peptides #49 and #50, which displayed the highest signal intensities. The EspB amino acid sequence in the FIGURE is denoted by SEQ ID NO. 40.

FIG. 6B. shows mAb-EspB-B7 binding to EspB following pre-incubation with peptide #49 and peptide #49 scrambled.

FIG. 6C. shows mAb-EspB-B7 binding to EspB following pre-incubation with peptide #50 and peptide #50 scrambled.

FIG. 6D. shows mAb-EspB-B7 binding to EspB following pre-incubation with peptide #49+50. The binding was evaluated by competitive ELISA and detected using anti-human IgG HRP-conjugated antibody. Peptide #78 was used as a negative control. Error bars represent ±SD.

FIG. 7A-7D. mAb-EspB-B7 Binding to EspB Peptides

FIG. 7A. shows mAb-EspB-B7 binding to peptide #49 and peptide #49 scrambled (SEQ ID NO. 33, and 34, respectively).

FIG. 7B. shows mAb-EspB-B7 binding to peptide #50 and peptide #50 scrambled (SEQ ID NO. 35, 36, respectively).

FIG. 7C. shows mAb-EspB-B7 binding to peptide #49+50 (SEQ ID NO. 37). mAb-EspB-B7 binding to the various peptides was evaluated by ELISA. A 96 well plate was coated with the peptides before being incubated with serially diluted mAb-B7 and detected using anti-human IgG HRP-conjugated antibody. The Peptide #78 (SEQ ID NO. 38), was used as a negative control. Error bars represent +/−SD.

FIG. 7D. The table in the FIGURE shows sequences of peptides #49, #49 scrambled (SEQ ID NO. 33, 34, respectively), #50, #50 scrambled (SEQ ID NO. 35, 36, respectively), #49+50 and #78 (SEQ ID NO. 37, 38, respectively). Each peptide was synthesized with the addition of cysteine residues at the C and N-termini, to enable peptide cyclization.

FIG. 8A-8B. mAb-EspB-B7 Binds EspB Homologs in Other T3SS-Expressing Bacteria

FIG. 8A. Wild type and mutant EPEC, EHEC, C. rodentium and Salmonella were grown under T3SS-inducing conditions. EPEC, EHEC and C. rodentium mutant strains contain a deletion in the escN gene, while Salmonella contains a deletion in the invA gene, which results in non-functional T3SSs in these mutants. The bacterial cultures were centrifuged, and the supernatants were collected, normalized, and analyzed by SDS-PAGE and western blotting using mAb-EspB-B7.

FIG. 8B (8B-1, 8B-2, 8B-3). Amino acid sequence alignment of EspB from EPEC (SEQ ID NO. 40) with C. rodentium (SEQ ID NO. 46), EHEC (SEQ ID NO. 45), or Salmonella (SEQ ID NO. 47) EspB homologs. The dark bars and/or dots represent identical, amino acids in each corresponding sequence, the different residues are indicated. The mAb-EspB-B7 epitope is annotated above the amino acids that are part of the epitope.

FIG. 9A-9B. mAb-EspB-B7 Does not Inhibit EPEC Translocation Activity into HeLa Cells

FIG. 9A. Scheme of the effector translocation assay. Infection of HeLa cells with EPEC was monitored by detecting the degradation profile of JNK, a human kinase that is subjected to cleavage by the EPEC effector, NleD.

FIG. 9B. HeLa cells were infected with wild-type (WT) EPEC in the presence or absence of 400 nM mAb-EspB-B7. After 3 hr, cells were washed, and host cell proteins were extracted and subjected to western blot analysis using anti-JNK and anti-actin (loading control) antibodies. JNK and its degradation fragments are indicated at the right of the gel. Degradation of JNK was evident in the WT EPEC, sample but not in the uninfected sample or in the samples infected with EPEC ΔescN. HeLa cells infected with WT EPEC in the presence of 400 nM mAb-EspB-B7 showed a JNK degradation profile similar to that of WT EPEC in the absence of mAb-EspB-B7.

### FIG. 10A-10C. Electrochemical Chip Device

The FIGURE schematically illustrates an electrochemical chip device configuration for detection of cell (e.g., EPEC) suspension based on EIS techniques according to some possible embodiments.

FIG. 10A. shows the electrochemical chip device and a sample collector.

FIG. 10B shows an exploded view of the chip device.

FIG. 10c shows a sectional view of the Chip device.

### FIG. 11A-11B. Fabrication of Electrochemical Chips

FIG. 11A. shows process flow of chip fabrication by photolithography and sputtering: (a) The wafer is cleaned with acetone, isopropanol, and distilled water; (b) Photoresist (PR) coat is spun onto the wafer and soft baked. (c) Patterns are projected onto the wafer (photolithography); (d) the substrate is developed and unexposed PR is removed. (e) Titanium and gold are sputtered onto the substrate (f) the PR and gold are removed by a lift-off process. Following this, the wafer is rinsed with ACT, IPA, and DI, and (g) the wafer is ready for electroplating.

FIG. 11B (11B-1, 11B-2). Electroplating of reference electrodes. top: Silver (Ag) electroplating setup showing the wafer immersed in an Ag plating bath while a common pad contacts all (thirty one) electrodes to be plated. A silver plate is used as anode (11B-1). The silver chloride (AgCl) layer is anodically generated in HCl by chronoamperometry. Potential is fixed vs a commercial Ag/AgCl reference electrode and a Pt wire is used as counter electrode (11B-2).

### FIG. 12A-12B. Characterization of the Reference Electrode

FIG. 12A. Following fabrication (and surface characterization of the deposited electrodes), the reference electrodes are electroplated. The electroplating of silver yields a typical white luster deposit that appears, in a SEM analysis, as a homogenous crystalline deposit with dense Ag nuclei of ˜1 μm (Bar: 5 μm).

FIG. 12B. Verification of a newly formed Ag/AgCl reference electrode is carried out by measuring its potential versus a commercial reference electrode in varying electrolyte (KCl) concentrations. The response of the electrode is plotted against the log[KCl] such that any log change in KCl concentration is expected to yield a 59 mV potential difference, according to the Nernst equation. In practice, deviations from this value are expected to evolve from the nature of the measured electrode (an ‘open’ reference electrode), the quality differences, and experimental conditions (mainly varying distances between the measuring electrodes that affect solution resistance). The reference electrodes demonstrate a ‘Nernstian behavior’, close to the theoretical value. measurements were performed in triplicates. Error bars denote SD from the mean.

### FIG. 13A-13C. Characterization of the EC Cell

Verification of the whole cell is obtained cyclic voltammetry with the well-known redox couple ferricyanide.

FIG. 13A. shows CV at different scan rates with a solution of 20 Mm ferricyanide/ferrocyanide. Four different scan rates were used consecutively.

FIG. 13B. Corresponding analysis obtained from the biochip. The peak height increased as scan rate increased and was linearly proportional to the square root of the scan rate, showing the anodic peaks (top) and cathodic (bottom).

FIG. 13C. peak separation is relatively independent of scan rate. Error bars are the SD from the mean for triplicates.

### FIG. 14A-14B. Biofunctionalization of EC Chips

FIG. 14A. Immobilization of antibodies is based on covalent attachment using well-established gold-thiol chemistry. Antibodies were thiolated by using the thiolating reagent 2-iminothiolane hydrochloride (Traut's reagent), which reacts with primary amines (—NH₂) to introduce sulfhydryl (—SH) groups while maintaining charge properties similar to the original amino group. The reaction was optimized to obtain an average of ˜6—SH group per antibody.

FIG. 14B. Ellman assay using DTNB (left) was used to assess the thiolation efficiency. The reaction is monitored by a spectrophotometer.

### FIG. 15A-15F. Surface Characterization of Functionalized Electrodes

FIG. 15A-15D. Assessment of thiolated antibodies immobilization to the gold working electrode is carried out by fluorescence microscopy analysis. Thiolated Cy3-labeled antibody is incubated on the gold WE. As a control, a non-thiolated Cy3 antibody was used. Incubation is followed by rigorous rinsing of the electrodes.

FIG. 15E-15F. AFM image of gold working electrode surface before and after the covalent immobilization of thiol-modified antibodies.

FIG. 16. The determination process

The FIGURE illustrates cell suspension determination process according to possible embodiments. More specifically, the FIGURE shows a flowchart exemplifying EIS characterization process.

### FIG. 17A-17B. Impedance Spectra

FIG. 17A. shows fitting parameters of a typical mAb-modified electrode.

FIG. 17B. shows the fitting result of a typical mAb-modified electrode.

FIG. 18. Custom fabricated apparatus

Figure shows image of a machined PTFE apparatus providing electrical contacts to electrochemical chips and chambers for interrogating multiple samples.

FIG. 19. The device Scheme

The FIGURE shows a possible embodiments of an electrochemical chip device packaged in a chamber along with an inlet “rough” filter (2 μm) and an outlet fine filter (500 nm). Once a sample collector perforates the seal an integrated syringe plunger is operated, extracting bacteria cells from the sampler towards the measurement chamber. The microelectrode array is connected through pads that are perpendicular to the package and are inserted into a ‘dongle-like’ potentiostat device. The measurement is handled by e.g., a smartphone application displaying electrochemical impedance spectroscopy (EIS) readouts, which is also responsible for data acquisition and storage, and is potentially capable of uploading the results to a designated cloud (not shown).

### FIG. 20A-20E. Mab-EspB-B7-Based Impedimetric Biosensor

FIGURE shows schematically illustrates a biosensor (e.g., mAb-EspB-B7-based impedimetric biosensor), and cell suspension measurement conducted therewith, according to some possible embodiments;

FIG. 20A. demonstrates EIS-based detection of whole bacterial EPEC cells. In this non-limiting example electrochemical chips (with a working electrode e_{w} radius of about 0.3 mm, counter electrode e_{c} having radius of about 0.6 mm, and a square reference electrode eᵣ having surface area of about 0.25 mm², and respective contact pads 13w,13c,13r electrically connecting thereto) fabricated in/on a substrate (13) using microelectronic fabrication technologies and are subsequently modified with a thiolated mAb-EspB-B7 using thiol-gold chemistry. The electrodes e_{w},eᵣ,e_{c} are sealably enclosed inside an electrochemical cell structure, configured to receive a sample. The immobilization of mAb-EspB-B7 and capture of antigen affect the impedance measured between the underlying electrodes. An ETS measurement thus allow for the interrogation of the electrochemical system and separation of the individual components that affect the electrochemical cell circuit established by introducing the sample into the electrochemical cell (cᵢ). The generated Nyquist plot is fitted to an equivalent circuit from which the different resistance values are extracted (inset).

FIG. 20B. Shows the Nyquist plots obtained from measurements of a bare gold working electrode (bare GE), from the working electrode after the immobilization of mAb-EspB-B7 (GE+mAb) thereon, and the mAb-EspB-B7-coated working electrode after incubation with 250 μg/mL purified EspB protein (GE+mAb+EspB).

FIG. 20C. Shows relative R_{ct} (charge transfer resistance) values of purified EspB protein (1, 4, 10 and 250 μg/ml) demonstrating a dose-dependent increase in the detected R_{ct} values. Relative R_{ct} values are the means of the R_{ct} ratios (before and after antigen capture) calculated from 3-6 measurements. Error bars represent the ±SD.

FIG. 20D. Shows that the change in the detected R_{ct} values is exponentially dependent on EspB concentration.

In FIG. 20E. specific binding of WT EPEC cells is indicated, resulting in a larger contribution to R_{ct} compared with the ΔespB null strain. The percent change in R_{ct} ratios measured for EPEC WT and ΔespB was calculated and averaged from 20 repeats (five measurements each containing four samples) for each strain. The mean of the averaged ratios and the standard error of the mean were calculated.

FIG. 21. Electrochemical cell device

The FIGURE shows schematically illustrates an electrochemical cell device (cᵢ) with a potentiostat (PS) and connection thereof to a computer device (e.g., smartphone), demonstrating how the binding of the EPEC cells to the mAb-EspB-B7 coated working electrode affects the EIS measurements.

FIG. 22. Electrochemical cell device

The FIGURE shows modification of a gold electrode (or any other suitable electrically conducting metal or carbon, or other conductive polymeric material that can be used as a working electrode in an electrochemical setup) with anti-pathogenic E. coli monoclonal antibodies such as: anti-EspB or others specific mAb's, and the impedance response measured over a predefined frequency range, according to possible embodiments. The impedance spectra is fitted to an electric circuit (right). Specific binding of antigens affects certain circuit parameters and enable detection and quantification of the bound antigen.

FIG. 23A-23D. electrochemical cells chip device

These figures schematically exemplify selected configuration of electrochemical cells chip device configured with plurality of electrode arrangement according to some possible embodiments.

FIG. 23A shows a chip configuration (60) comprising a plurality of electrochemical cells (C₁,C₂, . . . Cₙ) and respective plurality of electronic circuitries (65) electrically connected thereto.

FIG. 23B shows a chip configuration (69) comprising a plurality of working electrodes (e₁,e₂, . . . eₙ) enclosed inside a single electrochemical cells (cᵢ) operated using a single electronic circuitry (65).

FIGS. 23C and 23D. show the exploded and assembled chip configuration configured with a plurality of working and reference electrode and a common counter electrode.

### DETAILED DESCRIPTION OF THE INVENTION

Before specific aspects and embodiments of the invention are described in detail, it is to be understood that this invention is not limited to particular methods, and experimental conditions described, as such methods and conditions may vary. It is also to be understood that the terminology used herein is for the purpose of describing particular embodiments only, and is not intended to be limiting, since the scope of the present invention will be limited only by the appended claims.

In recent years, advances in mAb discovery and production have ushered in the development of pathogen-specific mAb's to be used either per se as antibacterial drugs or to be integrated into various diagnostic platforms for the detection of specific pathogens. In the latter regard, the high affinity and specificity of mAbs are characteristics that can be exploited in diagnostic tools giving reduced false positive/negative results. Such tools could provide rapid and accurate identification of bacterial agents at POC, thus supporting better clinical management of patients and preventing the transmission of infectious diseases in the community.

The present disclosure describes a mAb raised against EspB, an essential component within the T3SS that is crucial for the infectivity of numerous Gram-negative bacteria, including EPEC. The results disclosed herein demonstrate that mAb-EspB-B7 binds EspB with nM affinity and high specificity. As commercial monoclonal antibodies against bacterial species, targeted mostly against common bacterial antigen such as the flagella or the bacterial Lipopolysaccharides (LPS), have been reported to have micromolar affinities [18], the mAb-EspB-B7 holds greater potential to allow efficient detection of bacterial pathogens due to its nM affinity.

The antibody binding to its EspB target was stable over a wide range of pH values, excluding acidic pH values, and across various salt concentrations. A reduced binding capacity was detected only under high salt concentrations (>250 mM), suggesting that the antibody-antigen binding interface is governed by electrostatic interactions. This idea is supported by the observation that the identified EspB epitope contains nearly 50% of charged amino acids, which might be involved in the antibody-antigen binding. mAb-EspB-B7 demonstrated a relatively high melting temperature, which was moderately elevated when the antibody was complexed with its antigen. This result suggests that EspB binding has a stabilizing effect on the antibody, as was previously reported for anti-ricin neutralizing antibody. Furthermore, the melting temperature profile of mAb-EspB-B7 showed three distinct events that probably correspond to the melting order of the C_{H}2 region, followed by the Fab and C_{H}3, as reported previously. This melting profile indicates that the mAb-EspB-B7 would be suitable for applications that require relatively high thermal stability. The rational for pinpointing EspB derived from the fact that EspB is getting exposed to the extracellular environment following EPEC entrance to the digestive system and in response to thermal and chemical signals [6]. Based on the number of T3SS complexes expressed on each bacteria and the predicted number of EspB subunits found in each T3SS complex, the inventors estimate that there are approximately 100 EspB molecules per each bacterial cell [7]. The present disclosure reports the development and characterization of mAb-EspB-B7 and further demonstrate its potential as a bio-recognition element in a reliable and easy to use electrochemical biosensor. The mAb-EspB-B7 demonstrated high specificity and affinity towards EspB, binding capacity to soluble EspB and in the context of whole bacteria, and high stability under a variety of conditions. These characteristics make mAb-EspB-B7 an excellent candidate to serve as an integral component of a mAb-based biosensor. Indeed, a biosensor based on mAb-EspB-B7 demonstrated excellent performance in recognizing both soluble EspB and in the context of the whole bacteria. Such a biosensor can be used as a powerful tool for more rapid, cost-effective, and sensitive assays that can identify infective agents at the point of care (POC).

Epitope mapping using the specially designed cyclic-peptide array of the present disclosure revealed that mAb-EspB-B7 binds mostly to a specific amino acid sequence located at positions 193-210 along the EspB sequence (SEQ ID NO. 39). In a previous study, it was shown that this region was not important for EspB-EspD interactions, a fact that was further corroborated by our observation that mAb-EspB-B7 does not disrupt the interaction between the two proteins. Moreover, the observation that mAb-EspB-B7 binds EspB as a component of the fully assembled T3SS complex supports the notion that the epitope of EspB is exposed and not buried within the EspB-EspD interface. It is noteworthy that the peptide array results also identified an additional region, corresponding to peptides #9-12 (SEQ ID NO. 48), that demonstrated mAb-EspB-B7 binding. This finding could perhaps suggest that the epitope recognized by mAb-EspB-B7 is conformational rather than linear. As the main epitope sequence (positions 193-210) is fully conserved in EPEC and C. rodentium, the lower similarity along this second region might provide an explanation for the reduced western blot signal that was observed for C. rodentium EspB (FIG. 8A). In addition, while mAb-EspB-B7 binding to a protein was observed in the supernatants of WT EHEC and C. rodentium, no binding was detected in the Salmonella supernatant. This result is in agreement with the presence of the epitope in EHEC and C. rodentium but not in Salmonella (FIG. 8B).

The ability of mAb-EspB-B7 to recognize and bind C. rodentium EspB is highly important, as it provides the scientific grounds for the use of the mAb-EspB-B7 antibody as diagnosis tool of mice infection model. In addition, while mAb-EspB-B7 did not demonstrate a reduction of bacterial infectivity in the ex vivo system, the inventors posit that examining it in a mouse model will provide a more comprehensive picture that will include the effect of the antibody in promoting certain activities of the immune system against bacteria, such as opsonization and phagocytic clearance. These activities may prevent the spread of the bacterial infection within the host body and induce a humoral response with serological memory that will shorten the infection duration, promote recovery and provide cellular and serological memory.

Another key aspect of mAb-EspB-B7 is its ability to bind both the secreted form of EspB and EspB as a component of the assembled T3SS complex within the bacterial cell. This finding provides further support for its potential as a diagnostic agent capable of detecting bacterial infections directly in clinical samples in a short time with high accuracy, as previously reported [19, 20].

Demonstrating the diagnostic potential of mAb-EspB-B7 in electrochemical biosensing is particularly interesting. Electrochemical biosensors are perfectly suited for POC diagnosis due to their inherently high sensitivity and direct electronic transduction. Direct electronic detection avoids the use of optics and light sources and allows for small form-factor devices. Moreover, bioelectrochemical sensing is indifferent to sample turbidity thus obviating the need for extensive sample purification steps. Finally, these devices are attractive since they are amenable for miniaturization and can be manufactured using conventional microelectronic fabrication techniques. The inventors developed a biochip, functionalized it with the specific mAb-EspB-B7, and applied a label-free, EIS-based detection of EspB or alternatively, EspB-presenting bacteria by simply incubating the sample for several minutes. This direct approach to electrode functionalization is advantageous compared to well-established self-assembled monolayer (SAM) generation methods since it involves a straightforward preparation and avoids a complete electrode passivation often achieved with SAM. As shown in the present disclosure, despite obvious limitations related to nonspecific adsorption and sample inhomogeneity, the biosensor provides a concentration-dependent signal that can be fit to an exponential function yielding a calibration curve. Nonlinear calibration curves have been previously reported in impedimetric biosensors [21, 22]. In addition, the inventors observed that the biosensor differentiates between T3SS-containing- and lacking-bacteria, thus providing a simple tool to detect pathogenic bacteria.

In the present disclosure, the mAb-EspB-B7 that binds with high affinity and selectivity to a T3SS-exposed protein, has been characterized and provided clear indication for using this antibody integrated into a miniaturized electrochemical biosensor to identify T3SS-containing bacteria. The mAb-EspB-B7 antibody may also be used in development of anti-bacterial drug. The present disclosure provides the use of this antibody as a part of high throughput diagnostic device, such as a portable standalone antibody-based biosensor described herein.

Therefore, in a first aspect, the present disclosure provides a biosensor chip device.

More specifically, a biosensor chip device usable for identifying and/or quantifying a target in a sample by electrochemical impedance spectroscopy (EIS) analysis.

In some embodiments, the biosensor chip device disclosed herein comprises a plurality of electrodes connectable to at least one electronic device. It should be noted that at least one of the electrodes is a working electrode, the working electrode is connected directly or indirectly to at least one target binding site and/or moiety. It should be noted that the target binding site and/or moiety specifically targets and binds the at least one target or any component thereof. Still further, in some embodiments, the plurality of electrodes is configured for electrochemical impedance spectroscopy (EIS) analysis of the sample.

In yet some further embodiments, the chip device includes an arrangement of two or more electrodes configured to be in contact with a sample, typically within a measurement chamber. One of the two or more electrodes carries one or more binding sites, e.g., carrying antibodies such as the above described mAb-EspB-B7. The electrode arrangement is connectable to an electronic device for providing selected voltage variations between the two or more electrodes, enabling EIS analysis of material in the sample. In some examples, the EIS analysis enables to determine data on one or more bacteria cells in accordance with binding of the bacteria cells to respective binding sites on the electrodes.

For example, in some embodiments the chip device comprises:
a substrate portion having a plurality of electrodes formed in an electrodes portion thereof, and at least one electronic circuitry (e.g., potentiostat circuitry) electrically connected to said electrodes. In some embodiments, at least one of the electrodes is connected directly or indirectly to at least one target binding site and/or moiety; and
a packaging assembly configured to sealably enclose the electrodes portion of the substrate and define a measurement chamber encompassing the electrodes.

It should be understood that in some embodiments the target binding moiety is attached, connected, comprised within, deposited, integrated into, printed onto the at least one working electrode. Thus, the working electrode in some embodiments, is connected to, attached to and/or carries at least one target binding moiety. Still further, in some embodiments, the target binding site and/or moiety may be connected directly to the working electrode, or alternatively, via at least one linker or any other linking moiety that may be any chemical entity or modification, or alternatively, any peptide linker. In some embodiments, where the target binding moiety is an antibody, attaching the antibody (binding moiety), to the working electrode involves gold-thiol chemistry, specifically, attaching the thiolated antibody to the electrode. Still further, in some specific and non-limiting embodiments, antibodies were thiolated by using the thiolating reagent 2-iminothiolane hydrochloride (Traut's reagent), which reacts with primary amines (—NH₂) to introduce sulfhydryl (—SH) groups while maintaining charge properties similar to the original amino group. The reaction was optimized to obtain an average of ˜6—SH group per antibody. Thus, in some embodiments, the working electrode is covalently attached to immobilized thiol-modified antibodies, that serve as a target binding moiety.

FIGS. 10A to 10C schematically illustrate an electrochemical chip device (10) configuration for detection of cells (e.g., EPEC) suspension based some embodiments of the present disclosure. The biochip (10) contains an electrochemical cell (cᵢ) configured for holding an arrangement of a micro-working electrode array in communication of sample to be inspected. the electrode array includes a plurality of two or more electrodes, typically including at least one working electrode (e_{w}), at least one reference electrode (eᵣ) and at least one counter electrode (e_{c}). The electrode array may be formed on a substrate (13) to simplify alignment and electrical connections. In some embodiments, the electrode array may be made of Polytetrafluoroethylene (Teflon) or Acetal homopolymer (Delrin) or polypropylene, or polymethyl methacrylate or polyimide or polyvinylidene fluoride or polystyrene or other thermoplastics or heat-resistant plastic materials. FIG. 10A shows the electrochemical chip device (10) and, a sample collector (12) usable for introducing a sample into the chip device (10). The portion of the substrate (13) carrying active end of the electrodes (e_{w},eᵣ,e_{c}) of the chip device (10) is packaged in a chamber (cᵢ) and electrical contacts of the electrodes are shown (13w,13r,13e). Generally, the working electrode is connected to, or carrying, one or more binding sites/moieties selected to interact with one or more bacteria cells as described hereinabove. At least the working electrode (e_{w}) may preferably be formed and/or coated by a layer of gold, to enable biofunctionalization thereof.

FIG. 10B illustrates an exploded view of the electrochemical chip device (10). In this example the measurement chamber (11c) is defined between a base portion (11b) to which the electrodes portion of the substrate (13) is fitted, and a cover portion (11v) configured to sealably attach over the electrodes' portion of the substrate. The cover (11v) comprises a cavity (11c) configured to enclose the electrodes and define the measurement chamber of the chip device (10), and a sample insertion opening (11p). The sample insertion opening may generally be sealably covered by a sealer (11r). The chamber may further comprise one or more filters along general flow of sample material between sample insertion opening (11p) and the measurement chamber (11c), and downstream of the measurement chamber toward optional output port (not specifically shown). The one or more filters may include an inlet filter (11x), generally configured to be a “rough” filter, e.g., having pores in a range between 1 μm and 5 μm, and an outlet filter (11y), generally configured to be a fine filter, e.g., having pores in range between 100 nm and 1000 nm. Generally, the “rough” filter (11x) is configured to separate the electrolyte-containing sample loading chamber from the measurement chamber where large objects, such as cell debris, are filtered out. For example, the rough filter (11x) may have pores with average size of 2 μm. The fine filter (11y) is generally configured to separate the measurement chamber from a reservoir and to filter all objects, organisms, molecules, or any entity, that may be associated with the measurement, thereby maintaining such objects within the measurement chamber (11c). In some examples, filter (11y) may have pores of average size of 500 nm.

FIG. 10C exemplifies insertion of sample into chip device (10) using sample collector (12). In this example, sample collector (12) is configured to perforate the sealer (11c) and introduce bacteria cells from the sampler into the chip device (10). The bacteria cells are transmitted into the measurement chamber (11c) enabling interaction of the bacteria cells with one or more binding sites/moieties on the working electrode (e_{w}). The electrode array (e_{w},eᵣ,e_{c}) is connectable to an electronic device through respective contact pads (11w,11r,11c) e.g., extending perpendicular to the package, for providing electrical current/voltage and enabling measurement of impedance between the electrodes. For example, contact pads (11w,11r,11c) may extend outside of chip device (10) enabling inserting of the contact pads end as a ‘dongle-like’ attachment to a selected electronic device for performing measurements. In some configurations, the electronic device is configured to provide potentiostat measurements, typically acting as potentiostat device. The electronic device may be connectable/operated by one or more processors and corresponding computer readable instructions. For example, in some embodiments, the electronic device may be connectable (using wired or wireless connection) to a hand-held electronic device (e.g., a smartphone) carrying computer readable instructions for performing electrochemical impedance spectroscopy (EIS) measurement using the electrode array (e_{w},eᵣ,e_{c}) and provide corresponding readouts. The electronic device may also include a user interface enabling presentation of EIS readout, as well as storage and/or network communication ports for storing the readout data and transmitting such data to remote systems for analyzing. The electronic device may also be responsible for data acquisition and storage e.g., using internal storage and/or remote/cloud storage.

Biofunctionalization of the working electrode (e_{w}) can be carried out using thiol chemistry. The mAbs are first thiolated by incubation with Traut's reagent at a molar ratio of 1:15 for 1 hour at room temperature followed by washing with 0.1 M phosphate buffer pH 5 to remove the unreacted reagent. Thiolated mAbs are then covalently immobilized onto the gold working electrodes (e_{w}) of the chips devices (10) by drop-casting after thoroughly cleaning the electrodes by immersing 20 min in a solution of 50 mM KOH and 25% H₂O₂ followed by thorough rinsing with Milli-Q water.

An example of electrode manufacturing process is illustrated in FIGS. 11A and 11B. FIG. 11A shows a process flow of chip fabrication by photolithography and sputtering, FIG. 11B shows Electroplating of reference electrodes. The electrochemical chip (or biochips) may be designed as a miniaturized electrochemical cell having a three-electrode configuration (e.g., including working, counter and reference electrodes). The chip was microfabricated on a p-doped Si/SiO₂ substrate (with 285 nm thermally grown oxide) by a combination of photolithography defining electrode patterns and metal deposition (e.g., by sputtering Ti/Au 10 nm/90 nm). The process is illustrated in FIG. 11A in a step-by-step fabrication of electrochemical chips. In the fabrication process the wafer may typically be cleaned (a) generally using acetone, isopropanol, and distilled water. A Photoresist (PR) coat is spun onto the wafer and soft baked (b). A selected electrode pattern is projected onto the wafer (c) by photolithography. Following the photolithography, the substrate is developed and unexposed (d). The electrode material, e.g., including titanium and gold layers is sputtered onto the substrate (e). The photoresist layer and excess metals are removed by a lift-off process (f). Following this, the wafer is rinsed with ACT, IPA, and DI, to provide the final electrode pattern ready for electroplating (g).

The wafer-scale fabrication may be directed for producing a selected number of chips on each wafer. In the illustrated example, the fabrication process yielded 31 chips. The chips are formed of a selected arrangement of electrodes (typically three electrodes), generally formed of gold and associated with contact pads. The working electrode diameter was 0.6 mm.

FIG. 11B shows electroplating of reference electrodes. In the top image the wafer is inserted into a silver (Ag) electroplating setup in an Ag plating bath while a common pad contacts all the electrodes that are to be plated. A silver plate was used as anode. In the bottom image, the silver chloride (AgCl) layer is anodically generated in HCl by chronoamperometry. Potential is fixed vs a commercial Ag/AgCl reference electrode and a Pt wire was used as counter electrode. The on-chip reference electrodes may generally be prepared by various other techniques. In some examples, the reference electrode may be formed of Ag/AgCl.

Following electrode production, the chips may be diced in accordance with desired arrangement selected for chip device (10) design. FIGS. 12A and 12B show characterization of the generated chips conducted using scanning electron microscopy. The characterization indicates quality of the electroplated Ag/AgCl reference electrode (RE), and of the entire cell and electrode array. The electron microscopy shows typical white luster deposit that appears, in a SEM analysis, as a homogenous crystalline deposit with dense Ag nuclei of ˜1 μm (Bar: 5 μm). Verification of a reference electrode was carried out by measuring its potential versus a commercial reference electrode in varying electrolyte (NaCl or KCl) concentrations. The RE potential demonstrated a linear dependence on the log of the electrolyte concentration, as expected, following the Nernst equation. Briefly, Verification of the newly formed Ag/AgCl reference electrode may be carried out by constructing a simple EC cell with the new RE used as indicator electrode having its potential checked versus a commercial Ag/AgCl (saturated) electrode with fixed potential. Varying concentrations of KCl solution are used in order to plot the RE response to a change in KCl concentration, according to Nernst equation. The measured potentials are plotted against log of KCl molar concentration. In theory, one should expect to obtain a potential difference of 59 mV for each log of KCl concentration. In practice, deviations from this value are expected to evolve from the nature of the measured electrode (an open reference electrode), the quality differences and experimental conditions (temperature, varying distances between the measured electrodes, which affect solution resistance, etc.).

FIGS. 13A to 13C exemplify cyclic voltammetry (CV) for a chip device as described above. FIG. 13A shows cyclic voltammetry in the presence of the electroactive redox couple ferrocyanide/ferricyanide using four different scan rates consecutively. Two measured parameters of interest on these i-E curves (cyclic voltammograms) are the ratio of peak currents, iₚₐ/i_{pc}, and the separation of peak potentials, Eₚₐ-E_{pc}. For a voltametric Nernstian wave with stable product, iₚₐ/i_{pc}=1 regardless of scan rate and diffusion coefficients. Deviation of the ratio iₚₐ/i_{pc} from unity is indicative of homogeneous kinetic or other complications in the electrode process. The peak current for a reversible process is given by the Randles-Sevick equation.

FIG. 13B shows linear peak heights increase proportional to square root of the scan rates. This agrees with the Randles-Sevick equation. FIG. 13C shows a peak separation for different scan rates, indicating that the peak separation was not significantly affected by the scan rate.

Following manufacturing of the chip device, the working electrode (e_{w}) thereof undergoes biofunctionalization to provide suitable binding sites for selected bacteria cells or other biological materials. FIGS. 14A-14B and FIGS. 15A to 15D, which are described in more details further below exemplify biofunctionalization and characterization of the working electrode according to some embodiments of the present disclosure.

FIG. 16 generally describes technique for characterization of sample impedance using EIS technique according to some embodiments of the present disclosure. FIG. 16 illustrates operational actions typically implemented by the electronic circuit connectable to the electrical contacts (13w,13r,13e) of the electrodes in accordance with EIS techniques. As shown, the technique includes applying a voltage probe signal S1, typically in a selected signal frequency, and monitoring current passing through the electrodes in response S2. Based on the amplitude and phase relation between voltage and current the technique include determining cell impedance response S3. This can be visualized using Nyquist plot associated with equivalent electronic circuit S4. The impedance is given by the general notation indicting v=Zi, all being functions of signal frequency. Generally, impedance of the cell depends on interaction between any binding site on the working electrode (e_{w}) and biological materials in the measurement chamber (11c). In accordance with impedance variations, typically visualized by Nyquist plot, the technique includes determining charge transfer resistance S5. Impedance signature, including generally resistance, capacitance, and inductance, i.e., real and imaginary portions of the impedance, provide a signature of cells in the sample S6. Generally, in accordance with the selected binding sites and/or target binding/recognition moieties, carried by the working electrode, this enables determining data on one or more target, for example, bacteria types in the sample based on interaction of the target with the respective binding sites.

FIG. 17A-17B show impedance measurement results. The measurement relates to a circuit having equivalent configuration of capacitors, resistors, and inductors. The impedance is illustrated within complex numbers plane including real portion of the impedance (Re(Z)) and imaginary portion of the impedance (Im(Z)).

As indicated above, the biosensor chip device as described herein may be configured as a chip array having two or more electrodes, where at least one electrode carries selected binding sites. The electrodes are places in communication with a sample enabling the use of EIS technique for determining data on one or more materials based on interaction thereof with the selected binding sites. The present disclosure further provides a combined measurement system configured of a plurality of measurement chambers suitable for analysis of one or more samples simultaneously and/or sequentially. FIG. 18 shows an image of a machined PTFE apparatus carrying an array of a plurality of measurement chambers, each includes an electrode arrangement as described above. The electrode arrangement provides electrical contacts for each measurement chamber and may include similar or different binding sites on the working electrode of each measurement chamber. This enables detection of bacteria in a plurality of samples.

FIG. 19 shows a further detailed view of an electrochemical chip device (10) according to some embodiments of the present disclosure. As shown, the electrochemical chip device (10) is generally formed by an electrode arrangement (13), carrying at least one working electrode and at least one counter electrode, and typically also at least one reference electrode. The measurement chamber (11c) may be defined using one or more filters (11x) as described above, as well as sample input port (111). Once, a sample collector (12) is placed at the input port, e.g., perforates the seal, the device may utilize a plunger (110) for introducing sample material into the measurement chamber (11c). The plunger is illustrated in FIG. 19 by a syringe (110), and may be integral to the chip device or connectable thereto. Plunger operation generally pushes liquids through the chamber (11c), extracting bacteria cells from the sampler towards the measurement chamber. Within the measurement chamber, the introduced bacteria may interact with one or more binding sites on the working electrode end located therein, Interaction between the bacteria and the binding sites (e.g., antibody) varies electrical characteristics between the working and counter electrodes, measurable using EIS technique. To this end, the electrode array may be connectable to an electronic device, exemplified in FIG. 19 by a smartphone device (20) carrying a USB stick potentiostat (65), for providing electrical signals in accordance with EIS technique. The electronic device may also include one or more processors, memory, and communication ports for providing voltage signals, determining current response between the electrodes and determining impedance variation of the circuit as described above. The electronic device thereby provides electrochemical impedance spectroscopy (EIS) readouts, store such results, transmit the results and/or provide further processing. FIG. 19 also illustrates a simplified potentiostat circuit scheme. As described hereinbelow, the chip device may include an array of measurement chambers associated with respective plurality of arrays of electrodes, where each array of electrodes is associated with a potentiostat, or all utilizing a common potentiostat and a multiplexer that directs signal between the different electrode arrays.

FIGS. 20A to 20E illustrate the use of mAb-EspB-B7 as binding site in electrochemical chip device as described herein. FIG. 20A illustrates binding of bacterial EPEC cells to mAb-EspB-B7 and respective Nyquist plot; FIG. 20B shows Nyquist plot measurements using bare electrode, electrode carrying mAb-EspB-B7 binding sites and detection in a sample containing purified EspB protein; FIG. 20C shows relative charge transfer resistances (R_{ct}) for samples containing different amounts of charge transfer resistance compared to reference electrodes and samples; FIG. 20D show an exponential fit (using log scale) between detected R_{ct} values and EspB concentration; and FIG. 20E illustrates changes in R_{ct} for specific binding of WT EPEC cells is indicated, resulting in a larger contribution to R_{ct} compared between EPEC WT and ΔespB samples. FIG. 20A illustrates the details of EIS-based detection of whole bacterial EPEC cells. In this non-limiting example, electrochemical chips as described herein interact with bacterial EPEC cells, thereby varying impedance response along the electrode array. In this example, the electrode array includes a working electrode e_{w} radius of about 0.3 mm, counter electrode e_{c} having radius of about 0.6 mm, and a square reference electrode eᵣ having surface area of about 0.25 mm², and respective contact pads 13w,13c,13r electrically connecting thereto. The working electrode is modified with a thiolated mAb-EspB-B7 using thiol-gold chemistry. The electrodes e_{w},eᵣ,e_{c} are enclosed inside an electrochemical cell structure, configured to receive a sample. The immobilization of mAb-EspB-B7 and capture of antigen affect the impedance measured between the underlying electrodes as shown in FIGS. 20B to 20E. As shown, an EIS measurement allows for the interrogation of the electrochemical system and separation of the individual components that affect the electrochemical cell circuit established by introducing the sample into the electrochemical cell (cᵢ). The generated Nyquist plot may be fitted to an equivalent circuit from which the different resistance values are extracted (illustrated in an inset in FIG. 20A). The Nyquist plots shown in Figured 20B were obtained by EIS measurements of a bare gold working electrode (bare GE), working electrode after the immobilization of mAb-EspB-B7 (GE+mAb) thereon, the mAb-EspB-B7-coated working electrode after incubation with 250 μg/mL purified EspB protein (GE+mAb+EspB). Variation between the Nyquist plots indicates the electrochemical effects of the binding sites and interaction thereof of materials in the sample, thus enabling characterization of the sample. A suitable one-dimensional parameter that can be extracted from the Nyquist plots, using equivalent circuit fitting, in the relative charge transfer resistance (R_{ct}) values. FIG. 20C shows measured R_{ct} values for different concentrations of purified EspB protein (1, 4, 10 and 250 μg/ml), reference sample using modified working electrode. This variation demonstrates a dose-dependent increase in the detected R_{ct} values. Relative R_{ct} values are the means of the R-ratios (before and after antigen capture) calculated from 3-6 measurements. Error bars represent the ±SD. The variation in R_{ct} was fitted to exponential formula as a function of EspB protein concentration as shown in FIG. 20D. This model provides a fit R² of 0.978 indicating good agreement with the results. FIG. 20E shows measurement of specific binding of WT EPEC cells. The specific binding is indicated by larger contribution to R_{ct} compared with the ΔespB null strain. The percent change in R_{ct} ratios measured for EPEC WT and ΔespB was calculated and averaged from 20 repeating measurements (five measurements each containing four samples) for each strain.

FIG. 21 schematically illustrates an electrochemical cell device (cᵢ) using electrode array and electronic circuit for EIS measurement. The working electrode carried binding sites formed of the mAb-EspB-B7 to provide selective binding to EPEC cells. This is illustrated in FIG. 21 as E. coli cells do not attach to the binding sites and therefor provide EIS measurement associated with working electrode coated by the mAb-EspB-B7 binding sites that do not interact with bacterial cells. Presence of EPEC cells result in suitable interaction varying the EIS results as shown in FIG. 20C.

FIG. 22 shows an arrangement of electrode arrays on a chip device (PCB) and modification of the working electrode with selected binding sites. The working electrode may be formed of gold, or any other suitable electrically conducting metal, carbon, or conductive polymeric material that can be used as a working electrode in an electrochemical setup. The working electrode is coated by anti-pathogenic E. coli monoclonal antibodies such as: anti-EspB or others specific mAb's. Inset image of FIG. 22 shows impedance response measured over a predefined frequency range, according to some embodiments. The impedance spectra is fitted to an electric circuit (right) to determine simplified parameter such as charge transfer resistance R_{ct}. Specific binding of selected antigens affects certain circuit parameters and enable detection and quantification of the antigen bound hereto.

FIGS. 23A to 23D illustrate various configuration of electrochemical cells chip devices and electrode arrangement thereof. FIG. 23A shows chip configuration having a plurality of electrochemical cells; FIG. 23B illustrates an arrangement of a plurality of working electrodes in a single electrochemical cell; FIGS. 23C and 23D illustrate components in exploded and assembled views.

As shown in FIG. 23A, the device 60 may be formed as a printed circuit (e.g., chip) including a plurality of individual electrochemical cells (C₁,C₂, . . . Cₙ). Each electrochemical cells (Cᵢ) includes at least working (e_{w}) and counter (e_{c}) electrodes and is shows to also include a reference electrode (Eᵣ). Generally, electrode arrangement of each cell is associated with respective electronic circuit represented by respective potentiostat circuitries (65) for applying EIS measurement technique therethrough. The different electrochemical cells (C₁,C₂, . . . Cₙ) may be placed within a common measurement chamber, where each cell carries different binding sites, or configured to be placed in separated measurement chambers to simultaneous analysis of different samples.

Thus, the multiple electrochemical cell arrays (C₁,C₂, . . . ,Cₙ) shown in FIG. 23A are formed on a common substrate (13), where each electrochemical cell (Cᵢ, where 0≤i≤n is an integer) includes individual working, reference and counter electrodes (e_{w},eᵣ,e_{c}). In such embodiments the “reader” circuitry can be implemented utilizing respective potentiostat circuitries (65) for each one of the electrochemical cells. The measurement data generated by the potentiostat circuitries (65) may be used in various processing technique. For example, the measurement data may be digitized by digitizer unit (60a) for processing using a processing unit (60u) to determine amounts of bacteria suspension over the mAbs coated working electrodes (e_{w}) in the different electrochemical cells. The determined results can be locally stored in the memory device (60m), and/or communicated (wirelessly or over data lines) to external system/device (not shown) by the interface unit (60i).

The chip configuration (69) illustrated in FIG. 23B utilizes a plurality of working electrodes (e₁,e₂, . . . eₙ) associated with a single electrochemical cells (cᵢ). the different working electrodes may carry respective one or more different binding sites and may be operated using a common electronic circuit (e.g., single potentiostat circuitry) (65), or using one or more different electronic circuits. When operating using a common circuit, the readout may be enables using a multiplexer device (60x) providing selective signal feed to the different working electrodes (e₁,e₂, . . . eₙ), enabling to differentiate between readout from the different electrodes. Similarly to FIG. 23A output EIS signals may be digitized (60a) and transmitted for processing by processor (60u) to provide indication of one or more bacteria in the sample. This configuration enables (multiplexed) sequential measurements of a sample for various different agents (different bacterial agents).

FIGS. 23C and 23D illustrate another chip configuration (69) including a plurality of working electrodes (e_{w}) a respective plurality of reference electrodes (eᵣ) and a common counter electrode (e_{c}). As seen, in this non-limiting example each reference electrode (eᵣ) is positioned adjacent its respective working electrodes (e_{w}), and the common counter electrode is positioned around the arrangement of the plurality of working electrodes (e_{w}) and reference electrode (eᵣ). The different working electrodes may be modified to carry similar or different binding sites in accordance with desired sample analysis profile.

The substrate (13) carrying the electrodes may be any insulating substrate. Generally, the respective electronic EIS circuitry (e.g., potentiostat circuitry) may be placed on the same substrate as the electrodes, or connectable thereto via contact pads. Accordingly, this configuration may be implemented as a printed circuit boards, foils or film on which the electrode arrangement is deposited. In some embodiments the substrate may be fabricated using a semiconductor (e.g., Silicon) substrate and conventional semiconductor production techniques to implement the circuitries and electrodes on/in the substrate.

Accordingly, in some embodiments, the present disclosure provides a biosensor chip carrying an electrode arrangement formed of at least two electrodes comprising at least one working electrode carrying at least one target binding site and/or moiety, and at least one reference electrode. The biosensor chip may be configured to place the electrodes within a measurement chamber to be in liquid communication with sample solution, for analysis of one or more agents within the sample solution that attach to the at least one target binding site and/or moiety. The biosensor chip is connectable to an electronic device for electrical analysis of impedance between the electrodes, thereby determining data on the one or more agents within the sample solution.

Generally, the EIS analysis describe above may refer to faradic current transmitted between the working electrode and another electrodes (e.g., counter electrode), passing through the sample solution. This current may vary in response with attachment of one or more agents within the sample solution to the working electrode, thereby adjusting charge transmission into the sample solution.

In some embodiments, the plurality of electrodes of the biosensor chip device of the present disclosure may comprise at least one working electrode, at least one counter electrode configured to introduce electrical currents into the measurement chamber, and at least one reference electrode for measuring electrical voltage between the at least one working electrode and the at least one reference electrode. In more specific embodiments, the at least one working electrode is connected directly or indirectly to at least one target binding site and/or moiety. The reference electrode may provide reference impedance data associated with electrical characteristics of the sample solution, while being generally invariant to the one or more agents within the sample solution that attach to the at least one target binding site and/or moiety of the working electrode.

In yet some further embodiments, the biosensor chip device of the present disclosure may further comprise at least one inlet for introducing the sample into the measurement chamber; and at least one inlet filter for selectively passing the sample from the inlet into said measurement chamber.

In some other embodiments, the chip device of the present disclosure may comprise an outlet formed in the packaging assembly and at least one outlet filter for selectively passing sample material from the measurement chamber to the outlet.

Still further, in some embodiments, the packaging assembly of the biosensor chip device of the present disclosure comprises a base portion configured to receive the electrodes portion of the substrate, and a cover portion having an open cavity and configured to sealably attach to the base portion over the electrodes portion of the substrate and define the measurement chamber by its open cavity.

In some embodiments, the at least one electronic device of the disclosed biosensor chip comprises a plurality of potentiostat circuitries, the chip device of the present disclosure may comprise a plurality of measurement chambers, each comprising at least three of the plurality of electrodes defining a working electrode, a reference electrode, and a counter electrode, and a respective plurality of potentiostat circuitries each of which electrically connected to the at least three electrodes of its respective measurement chamber. More specifically, when referring to a plurality of measurement chambers and/o to a plurality of electrodes and/or a plurality of potentiostat circuitries, it is meant that in some embodiments, at least 3, 6, 9, 12, 15, 18, 21, 24, 27, 30 or more, 60, 90, 120, 150, 180, 210, 240, 270, 300 or more.

In some embodiments, the plurality of electrodes in the measurement chamber comprises define a plurality of working electrodes, at least one reference electrode, and at least one counter electrode. Still further, the device may comprise an electronic circuit (e.g., potentiostat circuitry) and a multiplexer device configured to selective transfer signals measured by the plurality of working electrodes to the potentiostat circuitry.

In some embodiments, Biosensor measurements are based on Electrochemical Impedance Spectroscopy (EIS). The faradaic current response of a routinely employed redox couple (10 mM K₃Fe(CN)₆) found within the measurement buffer, is monitored by EIS. The impedance spectra are obtained with a potential amplitude of 5 mV at a frequency range between 100 kHz and 10 Hz. The charge transfer resistance (R_{ct}) values may be obtained by fitting the generated Nyquist plots to equivalent circuits. The percent change in charge transmission resistance R_{ct} ratios between the biofunctionalized electrodes and varying EspB concentrations may be determined in accordance with ΣR_{ct}(EspB)/R_{ct}(mAb)−1.

In some embodiments of the device of the present disclosure, a variety of different types of working electrode may be used. For example, the working electrode may be carbon electrode, including glassy carbon, activated carbon cloth electrode, carbon felt, platinized carbon cloth, plain carbon cloth etc. the working electrode may be made of any conductive metal, for example, gold, platinum or silver, or any other conductive material including polymeric materials. The counter electrode may be made of similar material as the working electrode, or of a selected different conductive material. The reference electrode may for example be saturated calomel electrode, may be an Ag/AgCl electrode. Furthermore, the electrodes may be of a screen-printed electrode which can be inserted into the vessel comprising the cells without the need to withdraw a sample and transport it into a separate electrochemical cell. The electrodes used in the device of the invention, to detect the target according to the methods of the present disclosure, may be reusable electrodes or disposable ones. Reusable electrodes may for example be electrodes made of glassy carbon in a disk or rod shape which are embedded in Teflon. Disposable electrodes may for-example be electrodes in the form of carbon paper, carbon cloth, carbon felts, or the screen-printed electrode of the kind noted above. According to some embodiments, the electrochemical cell is a three-electrode cell. According to other embodiments, the electrochemical cell is a two-electrode cell. According to some further embodiments, the electrochemical cells are provided as an array (i.e. chip) comprising a plurality of such cells i.e. a multi-well/multi-spot array where each well is of a nano-volume size.

The device of the present disclosure, or any system for measuring the electrical signal generated by the reaction product may further comprise a control module which may be a computer, electronic device/circuitry (e.g., a potentiostat) and may include one or more multiplexer modules for providing separation between plurality of measurement channels when used.

As indicated above, the biosensor chip disclosed herein is usable for identifying and/or quantifying a target in a sample. As used herein, in some embodiments, the target is any entity comprising a proteineous material recognized by the target binding site/entity of the disclosed biosensor chip. As used herein, proteineous material may comprise proteins, peptides and any amino acid sequence as disclosed herein after.

In yet some further embodiments, the target identified and/or quantified is a pathogen comprising at least one proteineous material recognized by the binding moiety of the working electrode of the disclosed device.

Still further, a target pathogen as used herein refers to any pathogenic agents include any pathogens, such as viruses, prokaryotic microorganisms, lower eukaryotic microorganisms, complex eukaryotic organisms, fungi, prions, parasites, yeasts, as well as toxins and venoms. Of particular relevance are bacterial pathogens. A prokaryotic microorganism includes bacteria such as Gram positive, Gram negative and Gram variable bacteria and intracellular bacteria. Examples of bacteria contemplated herein include the species of the genera Treponema sp., Borrelia sp., Neisseria sp., Legionella sp., Bordetella sp., Escherichia sp., Salmonella sp., Shigella sp., Klebsiella sp., Pseudomonas sp., Yersinia sp., Vibrio sp., Hemophilus sp., Rickettsia sp., Chlamydia sp., Mycoplasma sp., Staphylococcus sp., Streptococcus sp., Bacillus sp., Clostridium sp., Corynebacterium sp., Propionibacterium sp., Mycobacterium sp., Ureaplasma sp. and Listeria sp.

A lower eukaryotic organism includes a yeast or fungus such as but not limited to Pneumocystis carinii, Candida albicans, Aspergillus, Histoplasma capsulatum, Blastomyces dermatitidis, Cryptococcus neoformans, Trichophyton and Microsporum.

A complex eukaryotic organism includes worms, insects, arachnids, nematodes, amoeba, Entamoeba histolytica, Giardia lamblia, Trichomonas vaginalis, Trypanosoma brucei gambiense, Trypanosoma cruzi, Balantidium coli, Toxoplasma gondii, Cryptosporidium or Leishmania.

In some further embodiments, viral pathogen/s may be detected and/or quantified by the biosensor chip of the present disclosure. The term “viruses” is used in its broadest sense to include viruses of the families adenoviruses, papovaviruses, herpesviruses: simplex, varicella-zoster, Epstein-Barr, CMV, pox viruses: smallpox, vaccinia, hepatitis B, rhinoviruses, coronaviruses, retroviruses, zika virus, Ebola virus, hepatitis A, poliovirus, rubella virus, hepatitis C, arboviruses, rabies virus, influenza viruses A and B, measles virus, mumps virus, HIV, HTLV I and II. The term “fungi” includes for example, fungi that cause diseases such as ringworm, histoplasmosis, blastomycosis, aspergillosis, cryptococcosis, sporotrichosis, coccidioidomycosis, paracoccidio-idoinycosis, and candidiasis. The term “parasite” includes, but not limited to, infections caused by somatic tapeworms, blood flukes, tissue roundworms, ameba, and Plasmodium, Trypanosoma, Leishmania, and Toxoplasma species.

In some embodiments, the target detected and/or quantified by the chip device of the present disclosure is at least one pathogen expressing at least one component of the Type III Secretion System (T3SS).

Still further, in some embodiments, the chip device of the present disclosure comprises at least one target binding site and/or moiety that may be comprised within or comprises at least one antibody that recognizes and binds at least one proteineous component of any of the disclosed pathogens. In some embodiments, the chip device of the present disclosure comprises at least one target binding site and/or moiety that may be comprised within at least one antibody that recognizes and binds at least one component of the T3SS, or any combination or complex thereof. In certain specific embodiments, at least one antibody is used as a target binding site, such antibody or any functional fragments thereof is directly or indirectly immobilized in some embodiments to the at least one working electrodes. A wide range of Ab immobilization chemistries are applicable in the present disclosure, provided that they all must assure that the immobilized antibody strongly retained to the surface (the working electrode) in a functionally oriented fashion such that its antigen-binding sites are free to bind the antigen, that is the target discussed herein. Some include simply adsorption of the antibody onto the substrate after a prolonged incubation by passive adsorption. In yet some further embodiments, various functionalization and cross-linking strategies may be used, for example, those described by the present methods that include the direct covalent attachment of thiolated antibodies to a gold electrode surface. More specifically, the thiolation reaction is optimized to obtain an average of ˜6—SH group per antibody by tuning the ratio of reagent to antibody. This fine-tuning enables control of the level of thiolation and ensures that antibody molecules are introduced with a sufficient number of thiols allowing their immobilization. Antibodies are thiolated in order to obtain a firm immobilization via gold-sulfur covalent bond, as discussed in the experimental procedures.

Still further, in some embodiments, the target binding site or moiety in the biosensor chip device of the present disclosure, is according to certain embodiments, at least one antibody that specifically recognizes and binds at least one component of the Type III Secretion System (T3SS) of at least one bacteria. Specifically, T3SS of Enteropathogenic Escherichia coli (EPEC).

The “Type III Secretion System or T3SS” is a complex structure composed of several subunits, which in turn are made up of approximately 20 bacterial proteins. The proteins that make up the T3SS apparatus are termed structural proteins. Additional proteins called “translocators” serve the function of translocating another set of proteins into the host cell cytoplasm. The translocated proteins are termed “effectors,” since they are the virulence factors that affect the changes in the host cells, allowing the invading pathogen to colonize, multiply, and in some cases chronically persist in the host. Briefly, the T3SS apparatus consists of two rings that provide a continuous path across the inner and outer membranes, including the peptidoglycan layer. The inner membrane ring is the larger of the two coaxial rings, and protein components that make up the inner ring have been identified for a number of bacteria. The outer membrane ring is composed of the secretin protein family, which is also known to be involved in type 2 secretion and in the assembly of type IV bacterial pili. A needle-like structure associates with the outer membrane ring and projects from the bacterial surface. It varies in length among the different pathogens and, in the case of pathogenic Escherichia coli, is extended by the addition of filaments that are thought to facilitate attachment to the host cells through the thick glycocalyx layer. Effectors are thought to be transported through the hollow tube-like needle into the host cell through the pores formed in the host cell membrane by the translocator proteins. Translocators are usually conserved among the different pathogens possessing a T3SS and show functional complementarity for secretion and translocation, whereas the effectors are most often distinct, having unique functions suited to a particular pathogen's virulence strategy. However, effector homologues also exist among different T3SS-possessing bacteria.

Still further, in some embodiments, the antibody comprised in the chip device of the present disclosure recognizes at least one component of the T3SS, for example, at least one of the Enteropathogenic Escherichia coli (EPEC) secreted protein A (EspA), EPEC secreted protein B (EspB), and EPEC secreted protein D (EspD), or any fragments or peptides thereof, and any combination or complex thereof.

In some embodiments, the chip device of the present disclosure comprises at least one antibody that recognizes and binds the EspB protein, or any fragments or peptides thereof, or any complex thereof with EspD protein.

In some embodiments, an antibody useful as a target binding site in the diagnostic biosensor chip devices, kits and methods of the invention may bind the Escherichia Coli secreted protein B (EspB) expressed by the bacterium, or any fragments or peptides thereof. Thus, in some embodiments, the diagnostic biosensor chip devices, kits and methods disclosed herein are used for detecting EspB expressing bacteria.

Among the virulence factors comprising the T3SS of these bacteria are the secreted proteins (Esps). The Esp responsible for the syringe-like structure of T3SS is secreted protein A (EspA), which is the needle-shaped protein of approximately 25 kDa, while secreted proteins B [Escherichia coli-secreted protein B (EspB)] and D [Escherichia coli-secreted protein D (EspD)] are responsible for the pore structure assembled in the eukaryotic membrane. Escherichia coli-secreted protein B is approximately 37 kDa in size and forms the pore assembled “needle tip” in the host cell membrane together with EspD. Also, EspB participates in phagocytosis evasion and binding to eukaryotic cell myosin, inhibition of actin interaction, and damage to the microvilli. There are three variants of EspB, i.e., α, β, and γ, where the α variant is subdivided into 1, 2, and 3. Allele frequency studies have shown a EspB to be the most prevalent, followed by β EspB.

In some embodiment, the EspB protein comprises the amino acid sequence as denoted by SEQ ID NO: 40 (Accession number: WP_001091991.1), or any homologs or derivatives thereof. In some specific embodiments, the EspB protein is encoded by a nucleic sequence as denoted by SEQ ID NO: 41 (Accession number: AAB69980.1), or any homologs or derivatives thereof. In some further embodiments, the EspD protein comprises the amino acid sequence as denoted by SEQ ID NO: 42 (Accession number: WP_000935767.1), or any homologs or derivatives thereof. In some specific embodiments, the EspD protein is encoded by a nucleic sequence as denoted by SEQ ID NO: 43 (Accession number: CAI43861.1).

In some embodiments, the isolated antibody used in the diagnostic biosensor chip devices, kits and methods of the invention, specifically recognizes and binds an epitope comprising residues 185 to 250, specifically residues 190 to 215, more specifically, residues 193 to 210, of the EspB protein, specifically, the EspB protein that comprises the amino acid sequence as denoted by SEQ ID NO. 40. In yet some further embodiments, the epitope recognized by the antibody of the invention may comprise the amino acid sequence of TSAQKASQVAEEAADAAQ, or at least part thereof. In yet some further embodiments, the epitope recognized by the antibody of the invention may comprise the amino acid sequence as denoted by SEQ ID NO: 39.

In some specific embodiments, the chip device of the present disclosure comprises (optionally directly or indirectly immobilized therein) at least one antibody that recognizes and binds the EspB protein. In more specific and non-limiting embodiments, the antibody of the chip device of the present disclosure comprises a heavy chain complementarity determining region (CDRH) 1 comprising the amino acid sequence GFTFSHYA, as denoted by SEQ ID NO. 6, CDRH2 comprising the amino acid sequence INSNGDST, as denoted by SEQ ID NO. 10, CDRH3 comprising the amino acid sequence ARDRRAGYFDYW, as denoted by SEQ ID NO. 14, and a light chain complementarity determining region (CDRL) 1 comprising the amino acid sequence RDNIGKNY as denoted by SEQ ID NO. 22, a CDRL2 comprising the amino acid sequence RNN as denoted by SEQ ID NO. 26, and a CDRL3 comprising the amino acid sequence SAWDTSLNA as denoted by SEQ ID NO. 30, or any derivative, variant and biosimilar thereof. As used herein, the term “biosimilar” relates in some embodiments, to a biological product, for example, proteins such as antibodies, antibody fragments (for example, antigen binding portions) and fusion proteins. A protein biosimilar may have an amino acid sequence that has minor modifications in the amino acid structure (including for example deletions, additions, and/or substitutions of amino acids) which do not significantly affect the function of the polypeptide. The biosimilar may comprise an amino acid sequence having a sequence identity of 97 percent or greater to the amino acid sequence of its reference medicinal product, e.g., 97 percent, 98 percent, 99 percent or 100 percent. The biosimilar may comprise one or more post-translational modifications, for example, although not limited to, glycosylation, oxidation, deamidation, and/or truncation which is/are different to the post-translational modifications of the reference medicinal product, provided that the differences do not result in a change in safety and/or efficacy of the medicinal product. The biosimilar may have an identical or different glycosylation pattern to the reference medicinal product. Particularly, although not exclusively, the biosimilar may have a different glycosylation pattern if the differences address or are intended to address safety concerns associated with the reference medicinal product.

In some embodiments, the antibody may comprise a heavy chain complementarity determining region (CDRH) 1 comprising the amino acid sequence GFTFSHYA, as denoted by SEQ ID NO. 6, or any homologs or derivatives thereof, CDRH2 comprising the amino acid sequence INSNGDST, as denoted by SEQ ID NO. 10, or any homologs or derivatives thereof, CDRH3 comprising the amino acid sequence ARDRRAGYFDYW, as denoted by SEQ ID NO. 14, or any homologs or derivatives thereof, and a light chain complementarity determining region (CDRL) 1 comprising the amino acid sequence RDNIGKNY as denoted by SEQ ID NO. 22, or any homologs or derivatives thereof, a CDRL2 comprising the amino acid sequence RNN as denoted by SEQ ID NO. 26, or any homologs or derivatives thereof, and a CDRL3 comprising the amino acid sequence SAWDTSLNA as denoted by SEQ ID NO. 30, or any homologs or derivatives thereof, or any derivative, variant and biosimilar of the antibody of the invention. In some embodiments, the antibody may comprise a heavy chain variable region and a light chain variable region, specifically, comprising CDR sequences as described above. In some specific embodiments, the heavy chain variable region is encoded by a nucleic acid sequence which is at least 70% identical to the nucleic acid sequence denoted by SEQ ID NO. 1, or any homologs or derivatives thereof. In yet some further embodiments, the light chain variable region is encoded by a nucleic acid sequence which is at least 70% identical to SEQ ID NO. 17, or any homologs or derivatives thereof.

In some other embodiments, the antibody may comprise a heavy chain variable region comprising the amino acid sequence denoted by SEQ ID NO. 2 or any homologs, derivatives or variants thereof and a light chain variable region comprising the amino acid sequence denoted by SEQ ID NO. 18 or any homologs, derivatives or variants thereof.

In more specific embodiments, the isolated monoclonal antibody or any antigen-binding fragment thereof may comprise a Heavy chain Framework Region 1 (FR1) comprising the amino acid sequence denoted by SEQ ID NO: 4, or any homologs or derivatives thereof, a heavy chain FR2 comprising the amino acid sequence denoted by SEQ ID NO: 8, or any homologs or derivatives thereof and a heavy chain FR3 comprising the amino acid sequence denoted by SEQ ID NO: 12, or any homologs or derivatives thereof, and a Light chain Framework Region 1 (FR1) comprising the amino acid sequence denoted by SEQ ID NO: 20, or any homologs or derivatives thereof, a Light chain FR2 comprising the amino acid sequence denoted by SEQ ID NO: 24, or any homologs or derivatives thereof, and a Light chain FR3 comprising the amino acid sequence denoted by SEQ ID NO: 28, or any homologs or derivatives thereof.

The term “antibody” as used herein, means any antigen-binding molecule or molecular complex that specifically binds to or interacts with a particular antigen of any fragments thereof. The term “antibody” includes immunoglobulin molecules comprising four polypeptide chains, two heavy (H) chains and two light (L) chains inter-connected by disulfide bonds, as well as multimers thereof (e.g., IgM). Each heavy chain comprises a heavy chain variable region (abbreviated herein as HCVR or V_{H}) and a heavy chain constant region (CH). The heavy chain constant region comprises three domains, CH1, CH2 and CH3. Each light chain comprises a light chain variable region (abbreviated herein as LCVR or V_{L}) and a light chain constant region. The light chain constant region comprises one domain (CL1). The V_{H} and V_{L} regions can be further subdivided into regions of hypervariability, termed complementarity determining regions (CDRs), interspersed with regions that are more conserved, termed framework regions (FR). Each V_{H} and V_{L} is composed of three CDRs and four FRs, arranged from amino-terminus to carboxy-terminus in the following order: FR1, CDR1, FR2, CDR2, FR3, CDR3, FR4.

Typically, an antibody is composed of two immunoglobulin (Ig) heavy chains and two Ig light chains. In humans, antibodies are encoded by three independent gene loci, namely kappa (κ) chain (Igκ) and lambda (λ) chain (Igλ) genes for the Light chains and IgH genes for the Heavy chains, which are located on chromosome 2, chromosome 22, and chromosome 14, respectively.

The antibody of the invention may be a monoclonal antibody, and in some embodiments a humanized or human antibody or any antigen-binding fragment thereof. In some embodiments, the antibody of the invention is a monoclonal antibody. A monoclonal antibody, as used herein refers to an antibody produced by a single clone of cells or cell line producing identical antibody molecules. Monoclonal antibodies display monovalent affinity in binding the same epitope. It should be further understood that the present invention further encompasses any functional fragments of then antibody of the invention, such fragments are referred to herein as antigen binding fragments. The term “an antigen-binding fragment” refers to any portion of an antibody that retains binding to the antigen.

Non-limiting examples of antigen-binding fragments include: (i) Fab fragments; (ii) F(ab′)2 fragments; (iii) Fd fragments; (iv) Fv fragments; (v) single-chain Fv (scFv) molecules; (vi) dAb fragments; and (vii) minimal recognition units consisting of the amino acid residues that mimic the hypervariable region of an antibody (e.g., an isolated complementarity determining region (CDR)). Other engineered molecules, such as domain-specific antibodies, single domain antibodies, domain-deleted antibodies, chimeric antibodies, CDR-grafted antibodies, diabodies, triabodies, tetrabodies, minibodies, nanobodies (e.g. monovalent nanobodies, bivalent nanobodies, etc.), small modular immunopharmaceuticals (SMIPs), and shark variable IgNAR domains, are also encompassed within the expression “antigen-binding fragment,” as used herein.

Examples of antibody functional fragments include, but are not limited to a single-domain antibody (sdAb) which refers to an antibody fragment consisting of a single monomeric variable antibody domain. The first single-domain antibodies were engineered from heavy-chain antibodies found in camelids; these are called VHH fragments. Cartilaginous fishes also have heavy-chain antibodies (IgNAR, ‘immunoglobulin new antigen receptor’), from which single-domain antibodies called variable new antigen receptor antibody (V-NAR) fragments can be obtained.

An alternative approach is to split the dimeric variable domains from common immunoglobulin G (IgG) from humans or mice into monomers. Although most research into single-domain antibodies is currently based on heavy chain variable domains, nanobodies derived from light chains have also been shown to bind specifically to target epitopes. Thus, it should be further appreciated that in some embodiments, the invention further encompasses a polypeptide comprising a variable region of a light chain comprising at least one of the CDR comprising the amino acid sequences as denoted by SEQ ID NO. 22, 26 and 30, or any homologs or derivatives thereof. In yet some further embodiments, the polypeptide of the invention may comprise the sequence of a variable region, as denoted by SEQ ID NO. 18, or any homologs thereof. In yet some further embodiments, the invention further provides a polypeptide comprising a variable region of an antibody heavy chain. In some specific embodiments, such polypeptide may comprise the amino acid sequence of at least one of the following CDRs, specifically, CDRs comprising the amino acid sequences as denoted by any one of SEQ ID NO. 6, 10 and 14, or any homologs or derivatives thereof. In yet some further embodiments, the polypeptide of the invention may comprise the variable region of the heavy chain as denoted by SEQ ID NO. 2, or any homologs or derivatives thereof.

As appreciated by one of skill in the art, various antibody fragments can be obtained by a variety of methods, for example, digestion of an intact antibody with an enzyme, such as pepsin, or de novo synthesis. Antibody fragments are often synthesized de novo either chemically or by using recombinant DNA methodology. Thus, the term antibody, as used herein, includes antibody fragments either produced by the modification of whole antibodies, or those synthesized de novo using recombinant DNA methodologies (e.g., single chain Fv) or those identified using phage display libraries. The term antibody also includes multivalent antibodies, specifically, bivalent molecules, diabodies, triabodies, tetrabodies and the like.

References to “V_{H}” or a “VH” refer to the variable region of an immunoglobulin heavy chain, including an Fv, scFv, a disulfide-stabilized Fv (dsFv) or Fab. References to “V_{L}” or a “VL” refer to the variable region of an immunoglobulin light chain, including of an Fv, scFv, dsFv or Fab.

More specifically, the phrase “single chain Fv” or “scFv” refers to an antibody in which the variable domains of the heavy chain and of the light chain of a traditional two chain antibody have been joined to form one chain. Typically, a linker peptide is inserted between the two chains to allow for the stabilization of the variable domains without interfering with the proper folding and creation of an active binding site. A single chain antibody applicable for the invention, e.g., may bind as a monomer. Other exemplary single chain antibodies may form diabodies, triabodies, and tetrabodies.

It should be appreciated that in some embodiments, any antibody provided by the present disclosure and used by the diagnostic biosensor chip device, methods, and kits of the present disclosure is not a naturally occurring antibody. Specifically, any of the antibodies used herein cannot be considered as a product of nature. In yet some further embodiments, it should be noted that the epitope recognized by the antibodies of the invention may comprise, at least part of residues 185 to 250, specifically, residues 190 to 215, more specifically, 193 to 210 of the EspB protein, specifically, the EspB as denoted by SEQ ID NO. 40. Still further, in some embodiments, the antibody of the invention comprises at least part of the amino acid sequence TSAQKASQVAEEAADAAQ, as denoted by SEQ ID NO. 39. According to Donnenberg et al. (Donnenberg et a. (2011) Journal of Bacteriology; p 2972-2980), the EspB protein adopts a transmembrane topology with its C-terminus facing the host cytoplasm. Therefore, the epitope should be found inside the host cell following bacterial infection. It should be appreciated that the invention further encompasses in some embodiments thereof any antibody that recognizes and binds an epitope comprising the amino acid sequence as denoted by SEQ ID NO. 39, or any homologs or derivatives thereof.

The term “epitope” is meant to refer to that portion of any molecule capable of being bound by an antibody which can also be recognized by that antibody. Epitopes or “antigenic determinants” usually consist of chemically active surface groupings of molecules such as amino acids or sugar side chains and have specific three-dimensional structural characteristics as well as specific charge characteristics.

In yet some further embodiments, the antibody of the invention cannot be considered as naturally occurring antibody. As such, the antibody of the invention is not a product of nature. Still further, it should be understood, that in some embodiments thereof, the invention further encompasses the use of any antibody that competes with any of the antibodies disclosed herein, specifically, any antibody that competes with an antibody comprising at least one of the CDRs as denoted by SEQ ID NO. 6, 10, 14, 22, 26 and 30, or any homologs or derivatives thereof. In yet some further embodiments, the invention further encompasses any antibody that competes with an antibody comprising the variable heavy chain as denoted by SEQ ID NO. 2, or any homologs or derivatives thereof, and/or the variable light chain that comprises the amino acid sequence as denoted by SEQ ID NO. 18, or any homologs or derivatives thereof. In yet some further embodiments, the term “competes” as used herein refers to any competition that results in reduction, attenuation, decrease or inhibition of binding of at least one of, the binding of the antibody of the invention to its epitope.

The invention relates to the use of antibodies that are polypeptides comprising amino acid sequences. “Amino acid sequence” or “peptide sequence” is the order in which amino acid residues connected by peptide bonds, lie in the chain in peptides and proteins. The sequence is generally reported from the N-terminal end containing free amino group to the C-terminal end containing amide. Amino acid sequence is often called peptide, protein sequence if it represents the primary structure of a protein, however one must discern between the terms “Amino acid sequence” or “peptide sequence” and “protein”, since a protein is defined as an amino acid sequence folded into a specific three-dimensional configuration and that had typically undergone post-translational modifications, such as phosphorylation, acetylation, glycosylation, mannosylation, amidation, carboxylation, sulfhydryl bond formation, cleavage and the like.

It should be appreciated that the invention encompasses the use of any variant or derivative of the antibody of the invention and any antibodies that are substantially identical or homologue to the antibodies encoded by the nucleic acid sequence of the invention. The term “derivative” is used to define amino acid sequences (polypeptide), with any insertions, deletions, substitutions and modifications to the amino acid sequences (polypeptide) that do not alter the activity of the original polypeptides. By the term “derivative” it is also referred to homologues, variants and analogues thereof. Proteins orthologs or homologues having a sequence homology or identity to the proteins of interest in accordance with the invention, specifically antibodies described herein, may share at least 50%, at least 60% and specifically 65%, 70%, 75%, 80%, 85%, 90%, 91%, 92%, 93%, 94%, 95%, 96%, 97%, 98%, 99%, or higher, specifically as compared to the entire sequence of the proteins of interest in accordance with the invention, for example, any of the antibodies that comprise the amino acid sequence as denoted by any one of SEQ ID NO. 2 and 18, or any one of the CDRs of SEQ ID NO. 6, 10, 14, 22, 26 and 30. Specifically, homologs that comprise or consists of an amino acid sequence that is identical in at least 50%, at least 60% and specifically 65%, 70%, 75%, 80%, 85%, 90%, 91%, 92%, 93%, 94%, 95%, 96%, 97%, 98%, 99%, or higher to SEQ ID NO. 2 and 18 specifically, the entire sequence as denoted by SEQ ID NO. 2 and 18, or any one of the CDRs of SEQ ID NO. 6, 10, 14, 22, 26 and 30.

In some embodiments, derivatives refer to antibodies, which differ from the antibodies specifically defined in the present invention by insertions, deletions or substitutions of amino acid residues. It should be appreciated that by the terms “insertion/s”, “deletion/s” or “substitution/s”, as used herein it is meant any addition, deletion or replacement, respectively, of amino acid residues to the polypeptides disclosed by the invention, of between 1 to 50 amino acid residues, between 20 to 1 amino acid residues, and specifically, between 1 to 10 amino acid residues. More particularly, insertion/s, deletion/s or substitution/s may be of any one of 1, 2, 3, 4, 5, 6, 7, 8, 9, or 10 amino acids. It should be noted that the insertion/s, deletion/s or substitution/s encompassed by the invention may occur in any position of the modified peptide, as well as in any of the N′ or C′ termini thereof.

With respect to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologues, and alleles of the invention.

For example, substitutions may be made wherein an aliphatic amino acid (G, A, I, L, or V) is substituted with another member of the group, or substitution such as the substitution of one polar residue for another, such as arginine for lysine, glutamic for aspartic acid, or glutamine for asparagine. Each of the following eight groups contains other exemplary amino acids that are conservative substitutions for one another:
1) Alanine (A), Glycine (G);
2) Aspartic acid (D), Glutamic acid (E);
3) Asparagine (N), Glutamine (Q);
4) Arginine (R), Lysine (K);
5) Isoleucine (I), Leucine (L), Methionine (M), Valine (V);
6) Phenylalanine (F), Tyrosine (Y), Tryptophan (W);
7) Serine (S), Threonine (T); and
8) Cysteine (C), Methionine (M).

More specifically, amino acid “substitutions” are the result of replacing one amino acid with another amino acid having similar structural and/or chemical properties, i.e., conservative amino acid replacements. Amino acid substitutions may be made on the basis of similarity in polarity, charge, solubility, hydrophobicity, hydrophilicity, and/or the amphipathic nature of the residues involved. For example, nonpolar “hydrophobic” amino acids are selected from the group consisting of Valine (V), Isoleucine (I), Leucine (L), Methionine (M), Phenylalanine (F), Tryptophan (W), Cysteine (C), Alanine (A), Tyrosine (Y), Histidine (H), Threonine (T), Serine (S), Proline (P), Glycine (G), Arginine (R) and Lysine (K); “polar” amino acids are selected from the group consisting of Arginine (R), Lysine (K), Aspartic acid (D), Glutamic acid (E), Asparagine (N), Glutamine (Q); “positively charged” amino acids are selected form the group consisting of Arginine (R), Lysine (K) and Histidine (H) and wherein “acidic” amino acids are selected from the group consisting of Aspartic acid (D), Asparagine (N), Glutamic acid (E) and Glutamine (Q).

Variants of the antibodies of the invention may have at least 80% sequence similarity or identity, often at least 85% sequence similarity or identity, 90% sequence similarity or identity, or at least 95%, 96%, 97%, 98%, or 99% sequence similarity or identity at the amino acid level, with the protein of interest, such as the antibodies of the invention.

In some embodiments, the invention relates to a biosimilar derived from the mAb-B7 antibody described above.

In some embodiments, the chip device of the present disclosure is usable for detecting the presence of a pathogen expressing at least one T3SS component in a sample. In some embodiments, such pathogen is a bacterial pathogen. In yet some further embodiments, the at least one bacteria is at least one Multiple Drug Resistant (MDR) bacteria.

In more specific embodiments, the MDR bacteria is at least one of Enteropathogenic Escherichia coli (EPEC) and Enterohemorrhagic Escherichia coli (EHEC).

In some embodiments, a sample that may be used for the chip device of the present disclosure may be a biological sample or an environmental sample, as will be described herein after.

A further aspect of the invention relates to a kit comprising:

First (a), at least one biosensor chip device usable for identifying and/or quantifying a target in a sample by electrochemical impedance spectroscopy (ETS) analysis, the chip device comprising: a plurality of electrodes connectable to at least one electronic device. At least one of these electrodes is a working electrode, the working electrode is connected directly or indirectly to at least one target binding site and/or moiety. In some embodiments, the target binding site and/or moiety specifically targets and binds the at least one target or any component thereof. Still further, the plurality of electrodes is configured for electrochemical impedance spectroscopy (EIS) analysis of the sample.

In yet some further embodiments, the biosensor chip device of the kits disclosed herein may comprise an arrangement of two or more electrodes configured to be in contact with a sample within a measurement chamber and to be connectable to an electronic device for enabling EIS measurement between the electrodes; wherein at least one of said electrodes is connected directly or indirectly to at least one target binding site and/or moiety.

In some embodiments the arrangement of two or more electrodes may be carried by a substrate, such as printed circuit.

In some embodiments, the measurement chamber may be formed by a packaging assembly configured to sealably enclose said electrodes portion of the substrate and define a measurement chamber encompassing said electrodes.

In some embodiments, the kit of the present disclosure optionally further comprises at least one of: (b) at least one control sample and/or control standard value, and (c) instructions for use.

In some embodiments, the kit disclosed herein may comprise at least one biosensor chip device as defined by the present disclosure.

A further aspect of the present disclosure relates to a method for identifying and/or quantifying at least one target in a sample, the method comprising:
providing (and/or contacting) an electrode arrangement comprising at least one reference electrode and at least one working electrode within connection with the sample, wherein said at least one working electrode carries at least one target binding site and/or moiety, or is connected directly or indirectly to the at least one target binding site and/or moiety; applying voltage signal between said at said least one working electrode and said at least one reference electrode, and determining current response on the working electrode (e.g., between the working electrode and a counter electrode) for a selected number of one or more signal frequencies;
utilizing a relation between current response and voltage signal and determining electrical impedance between the working electrode and counter electrode; impedance variation being indicative of presence and concentration of said at least one target in said sample.

It should be noted that in some embodiments, voltage between the electrodes may be determined in response to current signal driven therebetween in one or more selected frequencies.

In some embodiments, the method further comprises using one or more computer processor for processing electrical impedance determined based on one or more voltage signal frequencies for determining charge transfer electrical resistance between the working and counter electrodes, and determining presence of said at least one target in said sample based on said charge transfer electrical resistance. Generally, presence of the at least one target may be determined in accordance with a look-up table and/or predetermined threshold limits selected in accordance with data on said sample and said at least one target.

Generally, the voltage signal may be in the form of alternating voltage signals, e.g., sinusoidal wave, having one or more selected frequencies. Electrical impedance between the electrodes may be determined in accordance with magnitude of current response and phase shift between the current response and the voltage signal.

Generally, the charge transfer electrical resistance may be determined in accordance with a electrical circuit model representing charge transfer between the electrodes, such electrical circuit may comprise capacitance model connected in parallel to inductance model and charge transfer electrical resistance model, thereby allowing to determine charge transfer electrical resistance in accordance with total impedance of the circuit.

In some embodiments, the target detected and/or quantified by the methods of the present disclosure is at least one pathogen expressing at least one component of the Type III Secretion System (T3SS).

In yet some further embodiments, at least one target binding site and/or moiety used by the methods of the present disclosure is comprised within at least one antibody that recognizes and binds at least one component of the T3SS, or any combination or complex thereof. In more specific embodiments, the antibody or any functional fragments thereof is immobilized to at least one of the working electrode/s used by the methods of the present disclosure.

In more specific embodiments, the antibody used by the disclosed methods recognizes at least one component of T3SS, for example, at least one of the Enteropathogenic Escherichia coli (EPEC) secreted protein A (EspA), EPEC secreted protein B (EspB), and EPEC secreted protein D (EspD), and any combination or complex thereof.

In yet some further embodiments, the antibody used by the disclosed method may be at least one antibody recognizes and binds the EspB protein, or any complex thereof with EspD protein. In more specific embodiments, the method of the present disclosure may use at least one antibody that recognizes and binds the EspB protein. In more specific embodiments, such antibody comprises a heavy chain complementarity determining region (CDRH) 1 comprising the amino acid sequence GFTFSHYA, as denoted by SEQ ID NO. 6, CDRH2 comprising the amino acid sequence INSNGDST, as denoted by SEQ ID NO. 10, CDRH3 comprising the amino acid sequence ARDRRAGYFDYW, as denoted by SEQ ID NO. 14, and a light chain complementarity determining region (CDRL) 1 comprising the amino acid sequence RDNIGKNY as denoted by SEQ ID NO. 22, a CDRL2 comprising the amino acid sequence RNN as denoted by SEQ ID NO. 26, and a CDRL3 comprising the amino acid sequence SAWDTSLNA as denoted by SEQ ID NO. 30, or any derivative, variant and biosimilar thereof. In some embodiments, the method disclosed herein is intended for detecting at least one pathogen in a sample. In some embodiments, such pathogen is a bacterial pathogen. In yet some further embodiments, the bacteria is at least one Multiple Drug Resistant (MDR) bacterium. The present disclosure therefore provides diagnostic biosensor chip devices, kits and methods for detecting T3SS expressing bacteria in a sample. It should be noted that the term “bacterium” or “bacteria” as used herein refers to any of the prokaryotic microorganisms that exist as a single cell or in a cluster or aggregate of single cells. In more specific embodiments, the term “bacteria” specifically refers to Gram negative bacteria, or a Gram-positive bacteria, specifically, a Gram negative bacteria. In some embodiments, the at least one bacterium referred herein may be a gram-negative bacteria.

A yet further aspect of the present disclosure relates to a method for identifying and/or quantifying at least one target in a sample. More specifically, the method comprising the following steps:

The first step involves contacting at least one sample with a plurality of electrodes comprising at least one working electrode and at least one reference electrode or any biosensor chip or kit comprising the electrodes. In some embodiments, the plurality of electrodes used by the methods of the invention may further comprise at least one counter electrode. It should be noted that the at least one working electrode is connected directly or indirectly to at least one target binding site and/or moiety.

The next step involves measuring electrical voltages between the at least one working electrode and the at least one reference electrode in response to electric currents of different frequencies applied by the at least one counter electrode.

The next step involves determining electrical impedances based on the measured electrical voltage and the electric currents applied at the different frequencies.

In the next step, determining a charge transfer electrical resistance based on the determined impedances is performed.

The following step involves determining presence of the target in the sample whenever the charge transfer electrical resistance determined in the previous step is greater than a predetermined threshold value.

In some embodiments, determining a charge transfer electrical resistance by the method of the invention as indicated above, may comprise determining an electrical circuit model equivalent to a circuitry defined by the electrodes and the sample based on the determined electrical impedances. Such electrical circuit model may be associated with capacitance, inductance and resistance parameters, where at least a portion of total resistance model is associated with said charge transfer electrical resistance.

Still further, in some embodiments of the methods of the present disclosure, the determining of the equivalent electrical circuit model comprises correlating Nyquist presentation of the electrical impedances determined at the different frequencies to Nyquist presentation of electrical impedances of the equivalent electrical circuit model.

In yet some further embodiments, the measurement chamber used by the methods of the present disclosure comprises a plurality of working electrodes, each connected directly or indirectly to at least one target binding site and/or moiety. More specifically, the method comprising determining a respective plurality of electrical impedances associated with at least some of the plurality of working electrodes, and determining the charge transfer electrical resistance based of the determined respective plurality of electrical impedances.

In some embodiments, the measurement chamber comprises a plurality of working electrodes, each connected directly or indirectly to at least one target binding site and/or moiety, and a respective plurality of reference electrodes. Still further, according to these embodiments, the method comprising determining a respective plurality of electrical impedances associated pairs of said working and reference electrodes, and determining the charge transfer electrical resistance based of the determined respective plurality of electrical impedances.

In some embodiments, the target detected and/or quantified by the methods of the present disclosure is at least one pathogen expressing at least one component of the Type III Secretion System (T3SS).

In yet some further embodiments, at least one target binding site and/or moiety used by the methods of the present disclosure is comprised within at least one antibody that recognizes and binds at least one component of the T3SS, or any combination or complex thereof. In more specific embodiments, the antibody or any functional fragments thereof is immobilized to at least one of the working electrode/s used by the methods of the present disclosure.

In more specific embodiments, the antibody used by the disclosed methods recognizes at least one component of T3SS, for example, at least one of the Enteropathogenic Escherichia coli (EPEC) secreted protein A (EspA), EPEC secreted protein B (EspB), and EPEC secreted protein D (EspD), and any combination or complex thereof.

In yet some further embodiments, the antibody used by the disclosed method may be at least one antibody recognizes and binds the EspB protein, or any complex thereof with EspD protein.

In more specific embodiments, the method of the present disclosure may use at least one antibody that recognizes and binds the EspB protein. In more specific embodiments, such antibody comprises a heavy chain complementarity determining region (CDRH) 1 comprising the amino acid sequence GFTFSHYA, as denoted by SEQ ID NO. 6, CDRH2 comprising the amino acid sequence INSNGDST, as denoted by SEQ ID NO. 10, CDRH3 comprising the amino acid sequence ARDRRAGYFDYW, as denoted by SEQ ID NO. 14, and a light chain complementarity determining region (CDRL) 1 comprising the amino acid sequence RDNIGKNY as denoted by SEQ ID NO. 22, a CDRL2 comprising the amino acid sequence RNN as denoted by SEQ ID NO. 26, and a CDRL3 comprising the amino acid sequence SAWDTSLNA as denoted by SEQ ID NO. 30, or any derivative, variant and biosimilar thereof.

In some embodiments, the method disclosed herein is intended for detecting at least one pathogen in a sample. In some embodiments, such pathogen is a bacterial pathogen. In yet some further embodiments, the bacteria is at least one Multiple Drug Resistant (MDR) bacteria.

The present disclosure therefore provides diagnostic biosensor chip devices, kits and methods for detecting T3SS expressing bacteria in a sample. It should be noted that the term “bacterium” or “bacteria” as used herein refers to any of the prokaryotic microorganisms that exist as a single cell or in a cluster or aggregate of single cells. In more specific embodiments, the term “bacteria” specifically refers to Gram negative bacteria, or a Gram-positive bacteria, specifically, a Gram negative bacteria. In some embodiments, the at least one bacterium referred herein may be a gram-negative bacteria.

While the Gram-positive bacteria are recognized as retaining the crystal violet stain used in the Gram staining method of bacterial differentiation, and appear to be purple-colored under a microscope, the Gram-negative bacteria do not retain the crystal violet, making positive identification possible. In other words, the term bacteria apply herein to bacteria with a thin peptidoglycan layer of their cell wall that is sandwiched between an inner cytoplasmic cell membrane and a bacterial outer membrane (Gram-negative).

In yet some other embodiments, the bacteria relevant to the antibody of the invention may be at least one Multiple Drug Resistant (MDR) bacteria.

As used herein, the term “resistance” is not meant to imply that the bacterial cell population is 100% resistant to a specific antibiotic compound, but includes bacteria that are tolerant of the antibiotics or any derivative thereof. More specifically, the term “bacterial resistance gene/s” refers to gene/s conferring about 100%, 90%, 80%, 70%, 60%, 50%, 40%, 30%, 20%, or 10% protection from an antibiotic compound, thereby reversing susceptibility and sensitivity thereof to said antibiotic compound.

Bacteria of particular interest may be any bacteria involved in nosocomial infections or any mixture of such bacteria. The term “Nosocomial Infections” refers to Hospital-acquired infections, namely, an infection whose development is favored by a hospital environment, such as surfaces and/or medical personnel, and is acquired by a patient during hospitalization. Nosocomial infections are infections that are potentially caused by organisms resistant to antibiotics. Nosocomial infections have an impact on morbidity and mortality, and pose a significant economic burden. In view of the rising levels of antibiotic resistance and the increasing severity of illness of hospital in-patients, this problem needs an urgent solution. Common nosocomial organisms include Clostridium difficile, methicillin-resistant Staphylococcus aureus, coagulase-negative Staphylococci, vancomycin-resistant Enterococci, resistant Enterobacteriaceae, Pseudomonas aeruginosa, Acinetobacter and Stenotrophomonas maltophilia.

The nosocomial-infection pathogens may be Gram-negative rod-shaped organisms (Klebsiella pneumonia, Klebsiella oxytoca, Escherichia coli, Proteus aeruginosa, Serratia spp.), Gram-negative bacilli (Enterobacter aerogenes, Enterobacter cloacae), aerobic Gram-negative coccobacilli (Acinetobacter baumanii, Stenotrophomonas maltophilia) and Gram-negative aerobic bacillus (Stenotrophomonas maltophilia, previously known as Pseudomonas maltophilia). Among many others Pseudomonas aeruginosa is an extremely important nosocomial Gram-negative aerobic rod pathogen.

“ESKAPE” pathogens may also be of particular interest. As indicated herein, these pathogens include but are not limited to Enterococcus faecium, Staphylococcus aureus, Klebsiella pneumoniae, Acinetobacter baumanii, Pseudomonas aeruginosa, and Enterobacter.

In further embodiments, the bacteria as referred to herein by the invention may include Yersinia enterocolitica, Yersinia pseudotuberculosis, Salmonella typhi, Pseudomonas aeruginosa, Vibrio cholerae, Shigella sonnei, Bordetella Pertussis, Plasmodium falciparum, Chlamydia trachomatis, Bacillus anthracis, Helicobacter pylori and Listeria monocytogens.

In some other specific embodiments, the bacteria referred herein may be a gram negative. In other specific embodiments, the target cells of interest may be any E. coli strain, specifically, any one of 0157:H7, enteroaggregative (EAEC), enterohemorrhagic (EHEC), enteroinvasive (EIEC), enteropathogenic (EPEC), enterotoxigenic (ETEC) and diffuse adherent (DAEC) E. coli.

In some further embodiments, the MDR bacteria detected by the diagnostic biosensor chip devices, kits and methods of the present disclosure may be least one of Enteropathogenic Escherichia coli (EPEC) and Enterohemorrhagic Escherichia coli (EHEC). Enteropathogenic Escherichia coli and EHEC are the main bacterial agents associated with diarrhea among children under 5 years old, and both pathogens are able to induce the A/E lesion.

In some embodiments, the MDR bacteria may be Enteropathogenic Escherichia coli (EPEC).

In some other embodiments, the MDR bacteria may be C. rodentium.

In more specific embodiments, the MDR bacteria is at least one of Enteropathogenic Escherichia coli (EPEC) and Enterohemorrhagic Escherichia coli (EHEC).

In some further embodiments, the antibody of the biosensor chip device, kits and methods of the present disclosure recognizes and binds at least one component of the T3SS of at least one MDR bacteria, and therefore provides the diagnosis of an MDR bacteria in a sample, or in a subject.

In some further embodiments, the MDR bacteria may be at least one of EPEC and EHEC. Specifically concerning the EPEC and EHEC bacteria, the hallmark of EPEC and EHEC-induced intestinal pathology is the attaching and effacing (A/E) lesion, whose formation depends on a T3SS encoded within the loci of enterocyte effacement (LEE) and the interplay of many T3SS effectors. Following intimate attachment of the bacteria to the intestinal epithelium, the brush border microvilli are disrupted (effacement), and the bacteria promote formation of actin pedestals that elevate the pathogen above the intestinal epithelium. To attach to the enterocytes, EPEC and EHEC utilize their T3SSs to inject the Translocated Intimin Receptor (Tir) into the host cell, where it inserts into the host cell membrane and binds to the bacterial outer membrane protein intimin. Binding of intimin to Tir induces Tir clustering, initiating a cascade of signaling events that leads to actin polymerization and pedestal formation. This ultimately results in the formation of the A/E lesion. EPEC Tir is tyrosine phosphorylated to recruit the Arp2/3 complex and drive actin polymerization, whereas EHEC Tir is not phosphorylated but, rather, relies on an additional T3SS effector, TccP/EspFU, for Arp2/3 recruitment. Successful pedestal formation requires downregulation of filopodia, which form in response to EPEC/EHEC infection, as well as disruption of the host microtubule network. The T3SS effectors Map (mitochondrion-associated protein), Tir, EspH (153), EspG, and EspG2 mediate these processes. This multifaceted approach allows A/E pathogens to coordinate the formation of A/E lesions and actin pedestals, providing them with a unique niche in the intestine of the infected host.

In some more specific embodiments, the T3SS recognized by the antibody used by the methods of the invention may be an MDR bacteria, in some specific embodiments, such bacteria may be Enteropathogenic Escherichia coli (EPEC).

In yet another embodiment, the bacteria may induce attaching and effacing (A/E) lesion in the subject.

In some further embodiments, the bacteria referred herein may be C. rodentium.

In some further embodiments, the antibody used in the biosensor chip device, kits and methods of the invention may recognize the EspB expressed by the bacteria, as specified above.

In some embodiments, the methods of the invention may use, and thus may be applicable for identifying and/or quantifying of a target in a biological sample or an environmental sample. The terms “sample”, “test sample” and “specimen”, “biological sample” are used interchangeably in the present specification and claims and are used in its broadest sense. They are meant to include both biological and environmental samples and may include an exemplar of synthetic origin. This term refers to any media that may contain the T3SS expressing bacteria and may include body fluids (urine, blood, milk, cerebrospinal fluid, rinse fluid obtained from wash of body cavities, phlegm, pus), samples taken from various body regions (throat, vagina, ear, eye, skin, sores), food products (both solids and fluids) and swabs taken from medicinal instruments, apparatus, materials), as well as substances in which controlled chemical reactions are being carried out. More specifically, according to certain embodiments, the method of the invention uses any appropriate biological sample. The term “biological sample” in the present specification and claims is meant to include samples obtained from any subject or environmental sources, for example, a mammal subject. It should be recognized that in certain embodiments a biological sample may be for example, blood cells, blood, serum, plasma, bone marrow, lymph fluid, urine, sputum, saliva, feces, semen, spinal fluid or CSF, the external secretions of the skin, respiratory, intestinal, and genitourinary tracts, tears, milk, any human organ or tissue, any sample obtained by lavage, optionally of the breast ducal system, plural effusion, sample of in vitro or ex vivo cell culture and cell culture constituents. In certain embodiment, the biological sample suitable for the method of the invention may be any one of serum, whole blood sample, urine, saliva, or any fraction or preparation thereof. In some embodiments, the sample applicable in the biosensor chip device, kits and methods of the invention may be either as naturally obtained from the tested subject or manipulated and prepared. In some embodiments, the body fluid samples may be concentrated samples. In yet some further embodiments, the serum samples may be diluted and as such, different sera concentrations may be used. In some further embodiments the serum concentration may range between about 0.01% and 100%, More specifically, 0.01%, 0.02%, 0.03%, 0.04%, 0.05%, 0.06%, 0.07%, 0.08%, 0.09%, 0.1%, 0.2%, 0.2%, 0.4%, 0.5%, 0.6%, 0.7%, 0.8%, 0.9%, 1%, 2%, 3%, 4%, 5%, 6%, 7%, 8%, 9%, 10%, 11%, 12%, 13%, 14%, 15%, 16%, 17%, 18%, 19% or 20%, 25%, 30%, 35%, 40%, 45%, 50%, 55%, 60%, 65%, 70%, 75%, 80%, 85, 90%, 95%, 100% or more. In more specific embodiment, the sample concentration may range between about 1% to about 20%, in yet some further particular embodiments, the sample concentration of the sample may be 5%.

It should be further noted that in some embodiments, the diagnostic biosensor chip device, kits and methods of the invention may be also applicable for environmental samples. Environmental samples include environmental material such as surface matter, earth, soil, water, air and industrial samples, as well as samples obtained from food and dairy processing instruments, apparatus, equipment, utensils, disposable and non-disposable items. These examples are not to be construed as limiting the sample types applicable to the present invention. The sample may be any media, specifically, a liquid media that may contain the T3SS expressing bacteria. Typically, substances and samples or specimens that are a priori not liquid may be contacted with a liquid media which is contacted with the biosensor chip device of the invention.

More specifically, by the term “food”, it is referred to any substance consumed, usually of plant or animal origin. Some non-limiting examples of animals used for feeding are cows, pigs, poultry, etc. The term food also comprises products derived from animals, such as, but not limited to, milk and food products derived from milk, eggs, meat, etc.

In some specific embodiments, the present invention encompasses samples of a substance, which is used as a drink. A drink or beverage is a liquid which is specifically prepared for human consumption. Non limiting examples of drinks include, but are not limited to water, milk, alcoholic and non-alcoholic beverages, soft drinks, fruit extracts, etc.

In yet some further embodiments, the method of the invention, that detects, identify and/or quantify at least one pathogen in a sample, is used for the diagnosis of an infectious condition caused by or associated with at least one T3SS expressing pathogen, in a subject. According to these embodiments, the sample used by be disclosed method is at least one sample of the subject.

The present disclosure therefore provides a powerful diagnostic tool for rapid diagnosis of patients suffering from infectious condition caused by, or associated with, at least one bacteria expressing at least one T3SS.

The clinical spectrum of disease caused by T3SS-containing pathogens is remarkably broad. Infection with enteropathogenic and enterohemorrhagic E. coli (EPEC and EHEC, respectively), Shigella, Salmonella, and Yersinia species results in intestinal disease. Yersinia pestis is the causative agent of plague. Salmonella serovar Typhi causes enteric fever. Bordetella causes whooping cough, while the opportunistic pathogen Pseudomonas aeruginosa can cause a variety of problems, including pneumonia, urinary tract infection, wound infection, septicemia, and endocarditis. Chlamydia trachomatis is a common sexually transmitted organism, and Chlamydia pneumoniae causes pneumonia and has been implicated in atherosclerotic disease of blood vessels. Burkholderia pseudomallei causes community-acquired bacteremia and pneumonia. Whether by a direct toxic mechanism or through induction of self-damaging host responses, the virulence of all of these bacteria utilizes T3SSs. Clearly, T3SSs are not restricted to a specific pathogen, tissue, host environment, clinical disease spectrum, or patient population.

In some further embodiments, the diagnostic methods of the invention may be applicable for diagnosing any infection associated with at least one of transient enteritis or colitis, cholecystitis, bacteremia, cholangitis, urinary tract infection (UTI), traveler's diarrhea, neonatal meningitis and pneumonia, or any conditions, symptoms or effects associated therewith.

Thus, in some specific embodiments, the biosensor chip device, kits and methods of the invention may be applicable for transient enteritis. The term “transient enteritis or colitis” relates to an inflammation of the small intestine. It is most commonly caused by food or drink contaminated with pathogenic microbes. Duodenitis, jejunitis and ileitis are subtypes of enteritis which are only localized to a specific part of the small intestine. Inflammation of both the stomach and small intestine is referred to as gastroenteritis. Signs and symptoms of enteritis are highly variable and vary based on the specific cause and other factors such as individual variance and stage of disease. Symptoms may include abdominal pain, cramping, diarrhoea, dehydration, fever, nausea, vomiting and weight loss.

In yet some further embodiments, the biosensor chip device, kits and methods of the invention may be applicable for Cholecystitis. As used herein, Cholecystitis is inflammation of the gallbladder. Symptoms include right upper abdominal pain, nausea, vomiting, and occasionally fever. Often gallbladder attacks (biliary colic) precede acute cholecystitis. Complications of acute cholecystitis include gallstone pancreatitis, common bile duct stones, or inflammation of the common bile duct.

In some further embodiments, the biosensor chip device, kits and methods of the invention may be applicable for Bacteremia. Bacteremia (also bacteraemia) refers to the presence of bacteria in the blood. Bacteria can enter the bloodstream as a severe complication of infections (like pneumonia or meningitis), during surgery (especially when involving mucous membranes such as the gastrointestinal tract), or due to catheters and other foreign bodies entering the arteries or veins (including during intravenous drug abuse). Transient bacteremia can result after dental procedures or brushing of teeth.

Bacteremia can have several important health consequences. The immune response to the bacteria can cause sepsis and septic shock, which has a high mortality rate. Bacteria can also spread via the blood to other parts of the body (which is called hematogenous spread), causing infections away from the original site of infection, such as endocarditis or osteomyelitis. Still further, in some embodiments, the biosensor chip device, kits and methods of the invention may be applicable for cholangitis. Ascending cholangitis, also known as acute cholangitis or cholangitis, is inflammation of the bile duct, usually caused by bacteria ascending from its junction with the duodenum (first part of the small intestine). It tends to occur if the bile duct is already partially obstructed by gallstones. Characteristic symptoms include yellow discoloration of the skin or whites of the eyes, fever, abdominal pain, and in severe cases, low blood pressure and confusion.

In yet some further embodiments, the biosensor chip device, kits and methods of the invention may be applicable for urinary tract infection. A urinary tract infection (UTI) is an infection that affects part of the urinary tract. When it affects the lower urinary tract it is known as a bladder infection (cystitis) and when it affects the upper urinary tract it is known as kidney infection (pyelonephritis). Symptoms from a lower urinary tract include pain with urination, frequent urination, and feeling the need to urinate despite having an empty bladder. Symptoms of a kidney infection include fever and flank pain usually in addition to the symptoms of a lower UTI. In some cases, the urine may appear bloody.

In certain embodiments, the biosensor chip device, kits and methods of the invention may be applicable for Traveler's diarrhea. Traveler's diarrhea (TD) is a stomach and intestinal infection. TD is defined as the passage of unformed stool (one or more by some definitions, three or more by others) while traveling. It may be accompanied by abdominal cramps, nausea, fever, and bloating. Occasionally bloody diarrhea may occur. Most travelers recover within four days with little or no treatment. About 10% of people may have symptoms for a week. Bacteria are responsible for more than half of cases. The bacteria enterotoxigenic Escherichia coli (ETEC) are typically the most common except in Southeast Asia, where Campylobacter is more prominent.

In yet some more embodiments, the biosensor chip device, kits and methods of the invention may be applicable for Neonatal meningitis. Neonatal meningitis is a serious medical condition in infants. Meningitis is an inflammation of the meninges (the protective membranes of the central nervous system (CNS)) and is more common in the neonatal period (infants less than 44 days old) than any other time in life and is an important cause of morbidity and mortality globally. Symptoms seen with neonatal meningitis are often unspecific that may point to several conditions, such as sepsis (whole body inflammation). These can include fever, irritability, and dyspnea. The only method to determine if meningitis is the cause of these symptoms is lumbar puncture (LP; an examination of the cerebrospinal fluid). The most common causes of neonatal meningitis is bacterial infection of the blood, known as bacteremia (specifically Group B Streptococci (GBS; Streptococcus agalactiae), Escherichia coli, and Listeria monocytogenes). Delayed treatment of neonatal meningitis may cause include cerebral palsy, blindness, deafness, and learning deficiencies.

In some embodiments, the biosensor chip device, kits and methods of the invention may be applicable for Pneumonia. Pneumonia is an inflammatory condition of the lung affecting primarily the small air sacs known as alveoli. Typically, symptoms include some combination of productive or dry cough, chest pain, fever, and trouble breathing. Bacteria are the most-common cause of community-acquired pneumonia (CAP), with Streptococcus pneumoniae isolated in nearly 50% of cases. Other commonly-isolated bacteria include Haemophilus influenzae in 20%, Chlamydophila pneumoniae in 13%, and Mycoplasma pneumoniae in 3% of cases; Staphylococcus aureus; Moraxella catarrhalis; Legionella pneumophila; and Gram-negative bacilli. A number of drug-resistant versions of the above infections are becoming more common, including drug-resistant Streptococcus pneumoniae (DRSP) and methicillin-resistant Staphylococcus aureus (MRSA).

It should be understood that the diagnostic methods disclosed by the invention may be further used for monitoring subjects treated with any therapeutic compound.

More specifically, the diagnostic methods of the invention may be further used form monitoring the extent of infection (or bacterial load) in the treated subject. For such monitoring purpose, the steps of the methods of the invention may be repeated at least one further time for at least one further sample obtained from the subject. In some embodiments, the sample is obtained in another time point and is therefore considered herein as a temporally separated sample. As indicated above, in accordance with some embodiments of the invention, in order to assess the patient condition, or monitor the disease progression, as well as responsiveness to a certain treatment, at least two “temporally-separated” test samples must be collected from the examined patient and compared thereafter in order to obtain the rate of change in the amount of bacteria between said samples, as reflected by the amount of T3SS component (e.g., the EspB protein) measured and determined by the biosensor chip device, kits and methods of the invention. In practice, to detect a change in at least one of these parameters between said samples, at least two “temporally-separated” test samples and preferably more must be collected from the patient.

This period of time, also referred to as “time interval”, or the difference between time points (wherein each time point is the time when a specific sample was collected) may be any period deemed appropriate by medical staff and modified as needed according to the specific requirements of the patient and the clinical state he or she may be in. For example, this interval may be at least one day, at least three days, at least three days, at least one week, at least two weeks, at least three weeks, at least one month, at least two months, at least three months, at least four months, at least five months, at least one year, or even more.

When calculating the rate of change in the amount of the detected T3SS component (e.g., EspB), one may use any two samples collected at different time points from the patient. In some embodiments, at least one of the samples may be obtained before the initiation of an anti-bacterial therapy, and at least one of the samples may be obtained after the initiation of such therapy. To ensure more reliable results and reduce statistical deviations to a minimum, averaging the calculated rates of several sample pairs is preferable. A calculated or average value of a negative rate of change in bacterial load, as reflected by the amount of the T3SS component (e.g., EspB) in the sample, indicates that the subject exhibits a beneficial response to the treatment; thereby monitoring the efficacy of a treatment.

The number of samples collected and used for evaluation of the subject may change according to the frequency with which they are collected. For example, the samples may be collected at least every day, every two days, every four days, every week, every two weeks, every three weeks, every month, every two months, every three months every four months, every 5 months, every 6 months, every 7 months, every 8 months, every 9 months, every 10 months, every 11 months, every year or even more.

Thus, by providing a diagnostic tool, the present disclosure further provides therapeutic methods involving a diagnostic step. The diagnostic steps therefore provide tailor made methods allowing monitoring the patient for the presence of the T3S pathogen, during the treatment.

Thus, a further aspect of the present disclosure relates to a method of treating, preventing, ameliorating, reducing or delaying the onset of an infection by at least one bacteria expressing at least one T3SS in a subject in need thereof. In more specific embodiments, the method comprising:

In step (a), classifying a subject as infected by the bacteria if the presence of at least one T3SS component is determined in at least one sample of the subject. In some embodiments, determination of the presence of the at least one T3SS component in the sample is performed by, and/or comprising the following steps:
contacting the at least one sample of the subject with a plurality of electrodes comprising at least one working electrode and at least one reference electrode. In some embodiments, the sample is also contacted with a third electrode, being at least one counter electrode, or any biosensor chip device or kit comprising these electrodes. In some embodiments, at least one working electrode is connected directly or indirectly to at least one target binding site and/or moiety;
measuring and/or determining electrical voltages between the at least one working electrode and the at least one reference electrode in response to electric currents of selected one or more different frequencies applied by the at least one counter electrode;
   determining electrical impedances based on a relation between the measured electrical voltage and the electric currents applied at the different frequencies;
   determining a charge transfer electrical resistance based on the determined impedances; and
   determining presence of the bacteria expressing at least one T3SS in said sample whenever said charge transfer electrical resistance is greater than a predetermined threshold value, thereby classifying said subject as infected by the bacteria.

The next step (b), involves administering to a subject classified as an infected subject in step (a), a therapeutically effective amount of at least one anti-bacterial agent.

In some embodiments, the determination of the presence of the at least one T3SS component in the sample is performed by the method as defined by the present invention.

In yet some other embodiments, the method of the invention may further comprise the step of administering to the subject detected as infected by at least one T3S expressing bacteria, a therapeutically effect amount of at least one anti-bacterial agent.

In some embodiments, the antibacterial agent may be at least one antibiotic agent or any combinations thereof.

In some specific embodiments, the combined diagnostic and therapeutic methods of the invention may be applicable for infections caused by MDR bacteria.

In some more specific embodiments, the bacteria referred to herein may be a gram-negative bacteria.

In yet some other embodiments, the bacteria may be at least one of EPEC and EHEC.

In other specific embodiments, the methods of the invention are applicable for infectious caused by Enteropathogenic Escherichia coli (EPEC).

In some embodiments, the infections relevant to the method of the invention may be associated with at least one of transient enteritis or colitis, cholecystitis, bacteremia, cholangitis, UTI, traveler's diarrhea, neonatal meningitis and pneumonia, or any condition, symptoms or effects associated therewith, as disclosed herein above.

As indicated herein, the invention provides therapeutic methods involving a diagnostic step using the diagnostic biosensor chip device, kits and methods of the present disclosure. A subject diagnosed as infected with at least one T3SS expressing bacteria is treated according to some embodiments of the invention with at least one anti-bacterial agent. It should be further noted that the present disclosure further provides kits comprising the diagnostic biosensor chip device and any associated reagents and kits thereof, and in addition, at least one therapeutic agent, for example, an anti-bacterial compound.

Such agents may include anti-bacterial agent, anti-fungal agent, growth factors, anti-inflammatory agents, vasopressor agents including but not limited to nitric oxide and calcium channel blockers, collagenase inhibitors, topical steroids, matrix metalloproteinase inhibitors, ascorbates, angiotensin II, angiotensin III, calreticulin, tetracyclines, fibronectin, collagen, thrombospondin, transforming growth factors (TGF), keratinocyte growth factor (KGF), fibroblast growth factor (FGF), insulin-like growth factors (IGFs), IGF binding proteins (IGFBPs), epidermal growth factor (EGF), platelet derived growth factor (PDGF), neu differentiation factor (NDF), hepatocyte growth factor (HGF), vascular endothelial growth factor (VEGF), heparin-binding EGF (HBEGF), thrombospondins, von Willebrand Factor-C, heparin and heparin sulfates, and hyaluronic acid.

The term “antimicrobial agent” as used herein refers to any entity with antimicrobial activity (either bactericidal or bacteriostatic), i.e. the ability to inhibit the growth and/or kill bacteria, for example Gram negative bacteria. An antimicrobial agent may be any agent which results in inhibition of growth or reduction of viability of a bacteria by at least about 10%, 20%, 30% or at least about 40%, or at least about 50% or at least about 60% or at least about 70% or more than 70%, for example, 75%, 80%, 85%, 90%, 95%, 100% or any integer between 30% and 70% or more, as compared to in the absence of the antimicrobial agent. Stated another way, an antimicrobial agent is any agent which reduces a population of microbial cells, such as bacteria by at least about 30% or at least about 40%, or at least about 50% or at least about 60% or at least about 70% or more than 70%, or any integer between 30% and 70% as compared to in the absence of the antimicrobial agent. In one embodiment, an antimicrobial agent is an agent which specifically targets a bacteria cell. In another embodiment, an antimicrobial agent modifies (i.e. inhibits or activates or increases) a pathway which is specifically expressed in bacterial cells.

An antimicrobial agent can include any chemical, peptide (i.e. an antimicrobial peptide), peptidomimetic, entity or moiety, or analogues of hybrids thereof, including without limitation synthetic and naturally occurring non-proteinaceous entities. In some embodiments, an antimicrobial agent is a small molecule having a chemical moiety. For example, chemical moieties include unsubstituted or substituted alkyl, aromatic or heterocyclyl moieties including macrolides, leptomycins and related natural products or analogues thereof. Antimicrobial agents can be any entity known to have a desired activity and/or property, or can be selected from a library of diverse compounds.

In yet some further embodiments, such antibacterial agents may be antibiotic agents. Still further, in some embodiments such antibiotic agent may be at least one beta-lactam antibiotic agent.

The term “β-lactam” or “β-lactam antibiotics” as used herein refers to any antibiotic agent which contains a β-lactam ring in its molecular structure. β-lactam antibiotics are a broad group of antibiotics that include different classes such as natural and semi-synthetic penicillins, clavulanic acid, carbapenems, penicillin derivatives (penams), cephalosporins (cephems), cephamycins and monobactams, that is, any antibiotic agent that contains a β-lactam ring in its molecular structure. They are the most widely-used group of antibiotics. While not true antibiotics, the β-lactamase inhibitors are often included in this group. β-lactam antibiotics are analogues of D-alanyl-D-alanine the terminal amino acid residues on the precursor NAM/NAG-peptide subunits of the nascent peptidoglycan layer. The structural similarity between β-lactam antibiotics and D-alanyl-D-alanine prevents the final crosslinking (transpeptidation) of the nascent peptidoglycan layer, disrupting cell wall synthesis. Under normal circumstances peptidoglycan precursors signal a reorganization of the bacterial cell wall and, as a consequence, trigger the activation of autolytic cell wall hydrolases. Inhibition of cross-linkage by β-lactams causes a buildup of peptidoglycan precursors, which triggers the digestion of existing peptidoglycan by autolytic hydrolases without the production of new peptidoglycan. As a result, the bactericidal action of β-lactam antibiotics is further enhanced. Generally, β-lactams are classified and grouped according to their core ring structures, where each group may be divided to different categories. The term “penam” is used to describe the core skeleton of a member of a penicillin antibiotic. i.e. a β-lactam containing a thiazolidine rings. Penicillins contain a β-lactam ring fused to a 5-membered ring, where one of the atoms in the ring is sulfur and the ring is fully saturated. Penicillins may include narrow spectrum penicillins, such as benzathine penicillin, benzylpenicillin (penicillin G), phenoxymethylpenicillin (penicillin V), procaine penicillin and oxacillin. Narrow spectrum penicillinase-resistant penicillins include methicillin, dicloxacillin and flucloxacillin. The narrow spectrum β-lactamase-resistant penicillins may include temocillin. The moderate spectrum penicillins include for example, amoxicillin and ampicillin. The broad spectrum penicillins include the co-amoxiclav (amoxicillin+clavulanic acid). Finally, the penicillin group also includes the extended spectrum penicillins, for example, azlocillin, carbenicillin, ticarcillin, mezlocillin and piperacillin. Other members of this class include pivampicillin, hetacillin, bacampicillin, methampicillin, talampicillin, epicillin, carbenicillin, carindacillin, tie ancillin, azlocillin, piperacillin, mezlocillin, mecillinam, pivmecillinam, sulbenicillin, clometocillin, procaine benzylpenicillin, azidocillin, penamecillin, propicillin, pheneticillin, cloxacillin and nafcillin.

β-lactams containing pyrrolidine rings are named carbapenams. A carbapenam is a β-lactam compound that is a saturated carbapenem. They exist primarily as biosynthetic intermediates on the way to the carbapenem antibiotics. Carbapenems have a structure that renders them highly resistant to β-lactamases and therefore are considered as the broadest spectrum of β-lactam antibiotics. The carbapenems are structurally very similar to the penicillins, but the sulfur atom in position 1 of the structure has been replaced with a carbon atom, and hence the name of the group, the carbapenems. Carbapenem antibiotics were originally developed from thienamycin, a naturally-derived product of Streptomyces cattleya. The carbapenems group includes: biapenem, doripenem, ertapenem, imipenem, meropenem, panipenem and PZ-601. β-lactams containing 2,3-dihydrothiazole rings are named penems. Penems are similar in structure to carbapenems. However, where penems have a sulfur, carbapenems have another carbon. There are no naturally occurring penems; all of them are synthetically made. An example for penems is faropenem.

β-lactams containing 3,6-dihydro-2H-1,3-thiazine rings are named cephems. Cephems are a subgroup of β-lactam antibiotics and include cephalosporins and cephamycins. The cephalosporins are broad-spectrum, semisynthetic antibiotics, which share a nucleus of 7-aminocephalosporanic acid. First generation cephalosporins, also considered as the moderate spectrum includes cephalexin, cephalothin and cefazolin. Second generation cephalosporins that are considered as having moderate spectrum with anti-Haemophilus activity may include cefaclor, cefuroxime and cefamandole. Second generation cephamycins that exhibit moderate spectrum with anti-anaerobic activity include cefotetan and cefoxitin. Third generation cephalosporins considered as having broad spectrum of activity includes cefotaxime and cefpodoxime. Finally, the fourth generation cephalosporins considered as broad spectrum with enhanced activity against Gram positive bacteria and β-lactamase stability include the cefepime and cefpirome. The cephalosporin class may further include: cefadroxil, cefixime, cefprozil, cephalexin, cephalothin, cefuroxime, cefamandole, cefepime and cefpirome. Cephamycins are very similar to cephalosporins and are sometimes classified as cephalosporins. Like cephalosporins, cephamycins are based upon the cephem nucleus. Cephamycins were originally produced by Streptomyces, but synthetic ones have been produced as well. Cephamycins possess a methoxy group at the 7-alpha position and include: cefoxitin, cefotetan, cefmetazole and flomoxef.

β-lactams containing 1, 2, 3, 4-tetrahydropyridine rings are named carbacephems. Carbacephems are synthetically made antibiotics, based on the structure of cephalosporin, a cephem. Carbacephems are similar to cephems but with a carbon substituted for the sulfur. An example of carbacephems is loracarbef. Monobactams are b-lactam compounds wherein the β-lactam ring is alone and not fused to another ring (in contrast to most other β-lactams, which have two rings). They work only against Gram negative bacteria. Other examples of monobactams are tigemonam, nocardicin A and tabtoxin.

β-Iactams containing 3,6-dihydro-2H—I, 3-oxazine rings are named oxacephems or clavams. Oxacephems are molecules similar to cephems, but with oxygen substituting for the sulfur. Thus, they are also known as oxapenams. An example for oxapenams is clavulanic acid. They are synthetically made compounds and have not been discovered in nature. Other examples of oxacephems include moxalactam and flomoxef. Another group of β-lactam antibiotics is the β-lactamase inhibitors, for example, clavulanic acid. Although they exhibit negligible antimicrobial activity, they contain the β-lactam ring. Their sole purpose is to prevent the inactivation of β-lactam antibiotics by binding the β-lactamases, and, as such, they are co-administered with β-lactam antibiotics. β-lactamase inhibitors in clinical use include clavulanic acid and its potassium salt (usually combined with amoxicillin or ticarcillin), sulbactam and tazobactam.

It should be appreciated that the present disclosure may further provides a diagnostic-therapeutic kit. Thus, in some embodiments, the biosensor device of the present invention may be provided in a kit together with at least one anti-bacterial agent (e.g. an antibiotic agent) that may provide means for the combined diagnostic and therapeutic method encompassed by the invention. The kit of the present invention may, if desired, be presented in a pack which may contain one or more units of the kit of the present invention.

The terms “treat, treating, treatment” as used herein and in the claims mean ameliorating one or more clinical indicia of disease activity by administering a pharmaceutical composition of the invention in a patient having a pathologic disorder.

The term “treatment” as used herein refers to the administering of a therapeutic amount of the composition of the present invention which is effective to ameliorate undesired symptoms associated with a disease, to prevent the manifestation of such symptoms before they occur, to slow down the progression of the disease, slow down the deterioration of symptoms, to enhance the onset of remission period, slow down the irreversible damage caused in the progressive chronic stage of the disease, to delay the onset of said progressive stage, to lessen the severity or cure the disease, to improve survival rate or more rapid recovery, or to prevent the disease form occurring or a combination of two or more of the above.

The term “prevention” as used herein, includes the prevention or postponement of development of the disease, prevention or postponement of development of symptoms and/or a reduction in the severity of such symptoms that will or are expected to develop, preventing the occurrence or reoccurrence of the acute disease attacks. These further include ameliorating existing symptoms, preventing—additional symptoms and ameliorating or preventing the underlying metabolic causes of symptoms.

The term “amelioration” as referred to herein, relates to a decrease in the symptoms, and improvement in a subject's condition brought about by the compositions and methods according to the invention, wherein said improvement may be manifested in the forms of inhibition of pathologic processes associated with the infectious disease caused by a T3SS expressing MDR bacteria described herein, a significant reduction in their magnitude, or an improvement in a diseased subject physiological state.

The term “inhibit” and all variations of this term is intended to encompass the restriction or prohibition of the progress and exacerbation of pathologic symptoms or a pathologic process progress, said pathologic process symptoms or process are associated with.

The term “eliminate” relates to the substantial eradication or removal of the pathologic symptoms and possibly pathologic etiology, optionally, according to the methods of the invention described below.

The terms “delay”, “delaying the onset”, “retard” and all variations thereof are intended to encompass the slowing of the progress and/or exacerbation of a pathologic disorder or an infectious disease and their symptoms slowing their progress, further exacerbation or development, so as to appear later than in the absence of the treatment according to the invention.

More specifically, treatment or prevention include the prevention or postponement of development of the disease, prevention or postponement of development of symptoms and/or a reduction in the severity of such symptoms that will or are expected to develop. These further include ameliorating existing symptoms, preventing—additional symptoms and ameliorating or preventing the underlying metabolic causes of symptoms. It should be appreciated that the terms “inhibition”, “moderation”, “reduction” or “attenuation” as referred to herein, relate to the retardation, restraining or reduction of a process by any one of about 1% to 99.9%, specifically, about 1% to about 5%, about 5% to 10%, about 10% to 15%, about 15% to 20%, about 20% to 25%, about 25% to 30%, about 30% to 35%, about 35% to 40%, about 40% to 45%, about 45% to 50%, about 50% to 55%, about 55% to 60%, about 60% to 65%, about 65% to 70%, about 75% to 80%, about 80% to 85% about 85% to 90%, about 90% to 95%, about 95% to 99%, or about 99% to 99.9%.

With regards to the above, it is to be understood that, where provided, percentage values such as, for example, 10%, 50%, 120%, 500%, etc., are interchangeable with “fold change” values, i.e., 0.1, 0.5, 1.2, 5, etc., respectively.

It should be understood that the therapeutic methods of the invention involve any applicable mode of administration. The phrases “systemic administration”, “administered systemically” as used herein mean the administration of a compound, drug or other material other than directly into the central blood system, such that it enters the patient's system and, thus, is subject to metabolism and other like processes. The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal and intrasternal injection and infusion.

Systemic administration includes parenteral injection by intravenous bolus injection, by intravenous infusion, by sub-cutaneous, intramuscular, intraperitoneal injections or by suppositories, by patches, or by any other clinically accepted method, including tablets, pills, lozenges, pastilles, capsules, drinkable preparations, ointment, cream, paste, encapsulated gel, patches, boluses, or sprayable aerosol or vapors containing these complexes and combinations thereof, when applied in an acceptable carrier. Alternatively, to any pulmonary delivery as by oral inhalation such as by using liquid nebulizers, aerosol-based metered dose inhalers (MDI's), or dry powder dispersion devices.

By “topical administration” it is meant that the therapeutic methods disclosed herein may be adapted to any mode of topical administration including: epicutaneous, transdermal, oral, bronchoalveolar lavage, ophthalmic administration, enema, nasal administration, administration to the ear, administration by inhalation.

The invention provides methods for treating infectious diseases caused by bacterial infections. As used herein, “disease”, “disorder”, “condition” and the like, as they relate to a subject's health, are used interchangeably and have meanings ascribed to each and all of such terms.

It is understood that the interchangeably used terms “associated” and “related”, when referring to pathologies herein, mean diseases, disorders, conditions, or any pathologies which at least one of: share causalities, co-exist at a higher than coincidental frequency, or where at least one disease, disorder, condition or pathology causes a second disease, disorder, condition or pathology.

By “patient”, “individual” or “subject” it is meant any organism who may be affected by the above-mentioned conditions, and to whom the prognostic methods herein described are desired, including humans. More specifically, in some embodiments, the biosensor chip device, kits and methods disclosed herein, are applicable for any mammalian subject. By “mammalian subject” is meant any mammal for which the proposed therapy is desired, including human, equine, canine, and feline subjects, most specifically humans.

As used in this specification and the appended claims, the singular forms “a”, “an”, and “the” include plural references unless the context clearly dictates otherwise. Thus for example, references to “a method” includes one or more methods, and/or steps of the type described herein and/or which will become apparent to those persons skilled in the art upon reading this disclosure and so forth.

In yet some further aspect thereof, the present disclosure further provides at least one system comprising at least one biosensor chip device usable for identifying and/or quantifying a target in a sample by electrochemical impedance spectroscopy (EIS) analysis. More specifically, the at least one chip device of the disclosed system comprises: a plurality of electrodes connectable to at least one electronic device; wherein at least one of said electrodes is a working electrode, said working electrode is connected directly or indirectly to at least one target binding site and/or moiety. The target binding site and/or moiety specifically targets and binds the at least one target or any component thereof. The plurality of electrodes is configured for electrochemical impedance spectroscopy (EIS) analysis of said sample. In some embodiments, the system of the present disclosure may comprise any of the biosensor chip device disclosed by the present disclosure or any combinations thereof.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are now described.

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps. More specifically, the terms “comprises”, “comprising”, “includes”, “including”, “having” and their conjugates mean “including but not limited to”. This term encompasses the terms “consisting of” and “consisting essentially of”. The phrase “consisting essentially of” means that the composition or method may include additional ingredients and/or steps, but only if the additional ingredients and/or steps do not materially alter the basic and novel characteristics of the claimed composition or method.

The term “about” as used herein indicates values that may deviate up to 1%, more specifically 5%, more specifically 10%, more specifically 15%, and in some cases up to 20% higher or lower than the value referred to, the deviation range including integer values, and, if applicable, non-integer values as well, constituting a continuous range. As used herein the term “about” refers to ±10%.

It should be noted that various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible sub ranges as well as individual numerical values within that range. For example, description of a range such as from 1 to 6 should be considered to have specifically disclosed sub ranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range. Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals there between.

The following examples are put forth so as to provide those of ordinary skill in the art with a complete disclosure and description of how to make and use the methods and compositions of the invention, and are not intended to limit the scope of what the inventors regard as their invention. Efforts have been made to ensure accuracy with respect to numbers used (e.g., amounts, temperature, etc.) but some experimental errors and deviations should be accounted for. Unless indicated otherwise, parts are parts by weight, molecular weight is average molecular weight, temperature is in degrees Centigrade, and pressure is at or near atmospheric.

The examples are representative of techniques employed by the inventors in carrying out aspects of the present invention. It should be appreciated that while these techniques are exemplary of preferred embodiments for the practice of the invention, those of skill in the art, in light of the present disclosure, will recognize that numerous modifications can be made without departing from the spirit and intended scope of the invention.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub combination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

Disclosed and described, it is to be understood that this invention is not limited to the particular examples, methods steps, and compositions disclosed herein as such methods steps and compositions may vary somewhat. It is also to be understood that the terminology used herein is used for the purpose of describing particular embodiments only and not intended to be limiting since the scope of the present invention will be limited only by the appended claims and equivalents thereof.

### EXAMPLES

### Experimental Procedures

### Bacterial Strains

Wild-type (WT) EPEC O127:H6 strain E2348/69 [streptomycin-resistant] and EPEC null mutants (ΔescN, ΔespB, ΔespD) were used to purify EspB, to evaluate mAb-EspB-B7 binding, and to assess T3SS and translocation activities[8]. WT and T3SS-mutant strains of Citrobacter rodentium DBS100, enterohemorrhagic E. coli (EHEC), and Salmonella enterica serovar Typhimurium were used to assess antibody specificity. Antibiotics were used at the following concentrations: streptomycin (50 μg/mL), ampicillin (100 μg/mL), chloramphenicol (30 μg/mL), and nalidixic acid (50 μg/mL).

Expression and Purification of Recombinant EspB EPEC O127:H6 strain E2348/69 deleted for the espB gene (ΔespB) [8] was transformed with a bacterial expression vector encoding His-tagged EspB (EspB-His) and grown overnight in Luria-Bertani (LB) broth supplemented with the appropriate antibiotics. The following steps of the expression and purification of EspB are described herein. More specifically, the overnight culture was diluted 1:50 and grown for 3 hr under T3SS-inducing conditions (pre-heated Dulbecco's modified Eagle's medium [DMEM] in a tissue culture incubator with 5% CO2, statically). These conditions induce the secretion of EspB into the extracellular environment. Next, 0.5 mM isopropyl-β-d-thiogalactopyranoside (IPTG) was added, and the culture was grown for an additional 4 hr. The culture was centrifuged for 30 min at 12000×g, and the supernatant containing the secreted EspB-His was collected and supplemented with protease inhibitor cocktail of 200 mM phenylmethylsulfonyl fluoride (PMSF) and 1 μM benzamidine. The supernatant was then loaded on a His-Trap HP 1-mL column (GE Healthcare), washed with 50 mM imidazole, and eluted with 500 mM imidazole, according to the manufacturer's protocol. The elution fractions were analyzed by SDS-PAGE and Coomassie staining to identify the fractions that contain the purified protein. The recovered protein was further purified by gel filtration chromatography using a Superose 12 10/300 GL column (GE Healthcare). The peak fractions were collected, frozen in liquid nitrogen and stored at −80° C.
Phage Panning, mAb-EspB-B7 Expression and Purification

A human synthetic-phage library, displaying single-chain variable fragment (scFv), was used to isolate antibodies targeting EspB as described previously [9]. mAb-EspB-B7 V_{H} and V_{L} were cloned in mammalian expression vectors (pcDNA3.4H and pcDNA3.4 L encoding the IgG1 heavy and lambda light chain constant regions) by Gibson cloning. The cloned vectors were transformed into E. coli competent cells (XL-1 blue) and were purified using plasmid purification kit (Invitrogen). The vectors were co-transfected into Expi293 expression system (Gibco) according to the manufacturer's instructions. Transfected Expi293 cells were harvested by centrifugation at 2000×g for 10 min at 4° C. and conditioned medium was applied to MabSelect affinity column (GE Healthcare) according to the manufacturer's instructions.

### Enzyme-Linked Immunosorbent Assay (ELISA)

For all ELISA experiments, 96-well ELISA plates were coated with 5 μg/mL of target antigen in PBS and incubated overnight at 4° C. Blocking, washing and detection steps were carried out as described [10] previously. More specifically, EspB coated 96-well plates were blocked with 300 μL/well of 3% [w/v] skim milk in PBS for 1 hr at 37° C. and washed with PBS. mAb-EspB-B7 in blocking solution was added to the first line of the plate and serially diluted throughout the plate. The plate was incubated for 1 hr at room temperature, washed, and incubated with goat anti-human H+L HRP-conjugated secondary antibody in 0.05% PBST (Jackson ImmunoResearch) for 1 hr at room temperature. Plates were then washed and signal was developed using 3,3′,5,5′-tetramethylbenzidine (TMB). The reactions were quenched by 1 M H₂SO₄ and absorbance was measured at optical density (OD) of 450 nm (Epoch, BioTek). ELISA assays to test mAb-EspB-B7 binding in various conditions were carried out using similar protocol as described above with the following modifications: (i) for binding under various pH conditions, mAb-EspB-B7 was incubated in 0.1 M citric acid buffer pH 7.4, 7.0, 6.6, 5.6, and 4.6 during the binding step; (ii) for binding at various salt concentrations, mAb-EspB-B7 was incubated in 45.6 nM, 68.5 nM, 137 nM, 274 nM, and 411 nM NaCl; and (iii) for assessment of the serum effect on mAb-EspB-B7 binding, the antibody was incubated in 10% goat or horse serum with 1% Tween 20 and 1% human serum during the binding step. Competitive ELISA with peptides was carried out as follows: A 96-well ELISA plate (I) and a 96-well inert Bradford plate (II) were used for each of the peptides examined. The respective scrambled peptides (carrying the same amino acid compositions in a scrambled order), full-length EspB and peptide #78 (SEQ ID NO. 38) were used as positive and negative controls, respectively. Plate I was coated with 3 μg/ml EspB or PBS and incubated overnight at 4° C. Blocking of plate I was performed as described above. mAb-EspB-B7 (15 nM) was pre-incubated with serially diluted concentrations of peptides, starting at 15 μg/mL for 1 hr at room temperature, transferred to plate I, and incubated for 1 hr at room temperature. The remaining steps were performed as described above for regular ELISA.

### Surface Plasmon Resonance (SPR)

Association and dissociation of the EspB-mAb-EspB-B7 complex was monitored by SPR with a Biacore 200 apparatus (GE Healthcare Life Sciences) with EspB immobilized on a CM5 chip (GE Healthcare Life Sciences). SPR experiments were conducted according to the manufacturer protocols. Immobilization of EspB on CM5 chip was carried out by amine coupling chemistry using the following protocol at a flow rate of 10 μL/min and with 20 mM phosphate buffer with 0.15 M NaCl, and 0.005% Tween 20 at pH 5.91 as a running buffer. The chip was first activated by injecting a freshly prepared mixture of 50 mM N-hydroxysuccinimide and 195 mM 1-ethyl-3-(3-dimethylaminopropyl) carbodiimide for 7.5 min, then EspB (2.5 μg/mL in PBS buffer containing surfactant P20, 10 mM HEPES pH 7.4, 150 mM NaCl, and 3 mM EDTA) was injected for 5 min to reach 120 resonance units (RU), and finally the remaining activated carboxylic groups were blocked by injecting 1 M ethanolamine hydrochloride, pH 8.6, for 5 min. The association of mAb-EspB-B7 with EspB was monitored by injecting different concentrations of mAb-EspB-B7 for 4 min at a flow rate of 30 μL/min, and the dissociation was monitored at the end of the antibody injection. To regenerate the chip, 5 mM NaOH solution was used. Data analysis was carried out by fitting the sensorgrams to the steady state model (T200 evaluation software).

### In Vitro Type 3 Secretion Assay

In vitro T3SS assay was carried out as described previously for EPEC and Salmonella.

EPEC strains were grown overnight in LB supplemented with the appropriate antibiotics in a shaker at 37° C. The cultures were diluted 1:40 into pre-heated DMEM (Biological Industries) and grown statically for 6 hr in a tissue culture incubator (with 5% CO2), to an OD of 0.7 at 600 nm (OD600). These conditions simulate host environment and induce T3SS expression. The cultures were then centrifuged at 20,000×g for 5 min to separate the bacterial pellets from the supernatants; the pellets were dissolved in SDS-PAGE sample buffer, and the supernatants were collected and filtered through a 0.22-μm filter (Millipore). The supernatants were then precipitated with 10% (v/v) trichloroacetic acid (TCA) overnight at 4° C. to concentrate proteins secreted into the culture medium. The volume of the supernatants was normalized to the bacterial cultures at OD600 to ensure equal loading of the samples. The samples were then centrifuged at 18000×g for 30 min at 4° C., the precipitates of the secreted proteins were dissolved in SDS-PAGE sample buffer, and the residual TCA was neutralized with saturated Tris. The T3SS activity of C. rodentium was determined similarly to that described for EPEC. For EHEC, the inventors cultured double the amount of EPEC (8 mL cultures instead of 4 mL) due to lower amounts of secreted proteins of EHEC relative to EPEC.

### Immunoblotting

Samples were subject to immunoblotting as described previously [8]. Samples were subjected to SDS-PAGE and transferred to nitrocellulose membranes (pore size: 0.45 μm, Bio-Rad) or polyvinylidene difluoride (PVDF, Mercury, Millipore). The blots were blocked for 1 hr with 5% (w/v) skim milk-PBST (0.1% Tween in phosphate-buffered saline), incubated with the primary antibody (diluted in 5% skim milk-PBST for 1 hr at room temperature or overnight at 4° C.), washed, and then incubated with the secondary antibody (diluted in 5% skim milk-PBST, for 1 hr at room temperature). Chemi-luminescence was detected with EZ-ECL reagents (Biological Industries). The following primary antibodies were used: mAb-EspB-B7, diluted 1:1000; mouse anti-EspB (a gift from Prof. Finlay, University of British Columbia), diluted 1:1000; mouse anti-His (Pierce), diluted 1:2000; mouse anti-JNK (BD Pharmingen), diluted 1:1000 in TBS; and mouse anti-actin (MPBio), diluted 1:10,000. The following secondary antibodies were used: horseradish peroxidase-conjugated (HRP)-goat anti-mouse (Abcam Inc.) and HRP-conjugated goat anti-human (Abcam Inc) antibodies.

### Flow Cytometry

EPEC bacteria were grown overnight in LB with the appropriate antibiotics. The cultures were diluted 1:40 and grown under T3SS-inducing conditions for 3 hr. Thereafter, 1×10⁷ bacteria were plated in a 96-U shape well plate and centrifuged at 800×g for 5 min, and the supernatants were removed. Bacteria were incubated with primary antibody (mAb-EspB-B7, 1:100) for 1 hr at room temperature, washed with PBS, and stained using Alexa Fluor 488 goat anti-human IgG secondary antibody (Jackson ImmunoResearch) for 30 min. Samples were washed and resuspended in PBS for analysis. Flow cytometry analysis was performed on Gallios (Beckman Coulter) equipped with 488 nm, 405 nm and 638 nm lasers and a switchable 561 nm laser. Data analysis was performed with Kaluza software (Beckman Coulter).

### Co-Elution of EspB and EspD35-his by Nickel Affinity Chromatography

Co-elution assays were performed as previously described [8]. EPEC ΔespD in the presence or the absence of an EspD-³⁵His expression vector, was grown under T3SS-inducing conditions for 7 hr (0.5 mM IPTG was added after 3 hr to induce protein expression). To evaluate the ability of mAb-EspB-B7 to inhibit the interaction between EspB and EspD, 100 or 200 nM of mAb-EspB-B7 were added to EPEC ΔespD expressing EspD-35His sample. The supernatants, containing secreted EspD-³⁵His and EspB, were collected by centrifugation (20,000×g for 5 min) and were passed through a 0.45-μm-pore-size filter. Protease inhibitor solution was added to the samples (200 mM PMSF and 1 μM benzamidine), and they were incubated with Ni-NTA resin while being rotated overnight at 4° C. The samples were then loaded on gravity columns, and the flow-through was collected. The columns were washed three times with 5 mL of washing buffer (30 mM phosphate buffer pH 7.5, 500 mM NaCl, 50 mM imidazole), and proteins were eluted using elution buffer (30 mM phosphate buffer pH 7.5, 500 mM NaCl, 500 mM imidazole). Equal volumes of the supernatant and the eluate samples were precipitated with 10% (v/v) TCA for 1 hr at 4° C., centrifuged (30 min, 16000×g, 4° C.), air dried, and dissolved in SDS-PAGE sample buffer. Supernatants and eluted samples were analyzed by SDS-PAGE and western blotting using mouse anti-His and mouse anti-EspB antibodies, to avoid detection of the human mAB-EspB-B7 antibody.

### Epitope Mapping Using Peptide Array

Peptide microarrays of 15-residues cyclic peptides, derived from the EspB sequence and containing an overlap of 11 residues, were obtained from JPT Peptide Technologies GmbH. Peptide array analysis was carried out according to the manufacturer protocols. Each microarray included three identical subarrays as technical triplicates. Full-length EspB protein was spotted on the array and used as a positive control, while bovine serum albumin (BSA) served as a negative control. The binding of mAb-EspB-B7 to the peptide array was carried out according to the manufacturer's instructions (www.jpt.com), with minor modifications. Briefly, 20 μg/mL mAb-EspB-B7 (0.1% TBST v/v) were incubated on the peptide microarray for 2 hr at room temperature. The peptide microarray slides were then washed (five times with TBST), incubated with Alexa Fluor 647-affinipure mouse anti-human IgG (Jackson ImmunoResearch) for 45 min at room temperature, washed (five times with TBST and then five times with doubly distilled H2O), and dried. Fluorescence was detected with a GenePix 4000B scanner (Molecular Devices) at a resolution of 10 μm pixel size and analyzed by the Genepix Pro 6.0 analysis software (Molecular Devices). Signals were normalized and plotted to reflect the relative intensities of the fluorescence signals.

### Effector Translocation Activity

Translocation assays were performed as previously described. More specifically, HeLa cells (8×10⁵ cells per well) were infected for 3 hr with EPEC strains that were pre-induced for 3 hr for T3SS activity (pre-heated DMEM, statically, in a CO₂ tissue culture incubator). Cells were then washed with PBS, collected, and lysed with RIPA buffer. Samples were centrifuged at 18000×g for 5 min to remove non-lysed cells, and supernatants were collected, mixed with SDS-PAGE sample buffer, and subjected to western blot analysis with anti-JNK and anti-actin antibodies (loading control). Uninfected samples and the ΔescN mutant strain-infected samples were used as negative controls. To evaluate the ability of mAb-EspB-B7 to inhibit EPEC translocation activity, 400 nM of mAb-EspB-B7 were added to a sample infected with WT EPEC.

### Electrochemical Biosensor Fabrication

Electrochemical biochips were fabricated and biofunctionalized as previously reported [11, 12, 13]. More specifically, as shown by FIG. 10, electrochemical biochips were designed as electrochemical cells (cᵢ) with a three-electrode configuration (working electrode e_{w}, counter electrode e_{c} and a reference electrode eᵣ) and microfabricated on a p-doped Si/SiO₂ substrate (13, with 285 nm thermally grown oxide) by a combination of photolithography (to define the electrodes pattern) and sputtering (gold deposition, Ti/Au 10 nm/90 nm). The process flow showing the step-by-step fabrication of electrochemical chips is shown in FIG. 11A. The wafer-scale fabrication yielded 31 chips each comprising three gold electrodes (100 nm Au) as well as contact pads (13w,13r,13c). The working electrode diameter was 0.6 mm. On-chip Ag/AgCl reference electrodes (eᵣ) were prepared by electroplating (in an electroplating bath), as shown in FIG. 11B, and the individual chips were finally diced. The generated chips were characterized electrochemically and by scanning electron microscopy, as shown in FIG. 12A. The mAbs were thiolated by its incubation with Traut's reagent at a molar ratio of 1:15 for 1 hr at room temperature followed by washing with 0.1 M phosphate buffer pH 5 to remove the unreacted reagent. Thiolated mAbs were then covalently immobilized onto the gold working electrodes (e_{w}) of the chips by drop-casting after thoroughly cleaning the electrodes by immersing 20 min in a solution of 50 mM KOH and 25% H₂O₂ followed by thorough rinsing with Milli-Q water.

### Electrochemical Characterization of Chips

The quality of the electroplated Ag/AgCl quasi reference electrode (RE), and of the whole cell were electrochemically characterized. The RE potential demonstrated a linear dependence on the log of the electrolyte (KCl) concentration, as expected, following the Nernst equation (FIG. 12B).

An example of cyclic voltammetry (CV) for the EC biochip is presented in FIG. 13A, where four different scan rates were used consecutively. The peak heights increased with increasing scan rates and were linearly proportional to the square root of the scan rates (FIG. 13B), as expected, following the Randles-Sevick equation. In addition, the peak separation was not significantly affected by the scan rate (FIG. 13C).

### Biofunctionalization

Impedimetric immunosensors are based on immobilized antibodies to detect antigens using EIS on a solid-state electrode. The immobilization strategy of antibodies is of critical significance in the development because it determines the orientation of the antibody on the electrode's surface. The immobilization approach used in the present disclosure is based on the direct covalent attachment of thiolated antibodies to a gold electrode surface. The thiolation reaction was optimized to obtain an average of ˜6—SH group per antibody by tuning the ratio of reagent to antibody. This fine-tuning enables control of the level of thiolation and ensures that antibody molecules are introduced with a sufficient number of thiols allowing their immobilization. Estimation of introduced sulfhydryl groups was performed by Ellman assay that is used to quantify the number or concentration of thiol groups in a sample. Antibodies were thiolated in order to obtain a firm immobilization via gold-sulfur covalent bond. Traut's reagent (2-Iminothiolane, 2-IT) reacted with antibody primary amines to yield sulfhydryl groups, according to the mechanism shown in FIG. 14A. Estimation of introduced sulfhydryl groups was performed by Ellman assay. Ellman's Reagent (5,5′-dithiobis-(2-nitrobenzoic acid, or DTNB) is used to quantify the number or concentration of thiol groups in a sample. It is very useful as a sulfhydryl assay reagent because of its specificity for —SH groups at neutral pH, high molar extinction coefficient and short reaction time. DTNB reacts with a free sulfhydryl group to yield a mixed disulfide and 2-nitro-5-thiobenzoic acid (TNB). The target of DTNB in this reaction is the conjugate base (R—S⁻) of a free sulfhydryl group. TNB is the “colored” species produced in this reaction and has a high molar extinction coefficient with a value of 14,150 M⁻¹ cm⁻¹ at 412 nm. The DTNB reduction reaction and its structure are shown in FIG. 14B. Introduced —SH groups were quantified by reference to the extinction coefficient of TNB following:

C=A/bE; where A=absorbance, b=optical path length (cm), E=molar extinction coefficient, and C=concentration (molar). Antibodies incubated with Traut's reagent at a ratio of 1:10 and 1:15, yielded an average —SH groups per antibody of 3.63 and 6.7, respectively.

Gold surfaces can be readily reacted with the sulfur head of thiolated molecules enabling the immobilization of biorecognition molecules. An assessment of the immobilization efficiency was carried out by fluorescence microscopy analysis, using a fluorescently (Cy3)-labeled thiolated antibody compared with non-thiolated antibody. Fluorescence microscopy images shown in FIGS. 15A to 15D, confirm the immobilization of antibodies to the gold electrode. Electrode surface characterization by AFM, as shown in FIG. 15E to 15F, provides further indication for the immobilization of antibodies.

Finally, this direct approach to electrode functionalization is advantageous compared to well-established self-assembled monolayer (SAM) generation methods since it involves a straightforward preparation and avoids complete electrode passivation often attained with SAM. Furthermore, this functionalization procedure can be readily scaled up as it is compatible with microarray printing technology.

### Biosensor Measurements

Biosensor measurements were based on Electrochemical Impedance Spectroscopy (EIS) recorded by a commercial potentiostat device (BioLogic). The faradaic current response of a routinely employed redox couple (10 mM K₃Fe(CN)₆) found within the measurement buffer, was monitored both by cyclic voltammetry (CV) and EIS. The impedance spectra of the freshly cleaned electrodes were obtained prior and post antibody immobilization, with a potential amplitude of 5 mV at a frequency range of 100 kHz to 10 Hz. The CV was collected within a potential range of −200-600 mV vs. Ag/AgCl at a scan rate of 100 mV/sec. Purified EspB protein solutions (at the concentration of 0, 1, 4, 10, and 250 μg/mL in PBS) were incubated for 10 min on the working electrode and then measured by the EIS method. The specificity of the obtained signals was verified by two control experiments. A negative control that included an unrelated antigen (2 μg/ml of the toxin Microcystin-LR, PubChem CID: 445434) and additionally, a purified EspB antigen (2 μg/mL) without the immobilized mAb (on a nonfunctionalized bare electrode). All Measurements of soluble EspB were repeated 3-6 times for each protein concentration. The charge transfer resistance (R_{ct}) values were obtained by fitting the generated Nyquist plots to a Randles equivalent circuit. The percent change in R_{c}, ratios between the biofunctionalized electrodes and varying EspB concentrations was calculated and averaged from: ΣR_{ct}(EspB)/R_{ct}(mAb)−1.

In order to detect whole bacterial cell suspensions, EPEC WT and ΔespB mutant strains were cultured as described herein above, gently centrifuged (500×g, 5 min) and resuspended in PBS to a concentration of 3×10⁷ cells/mL. Five microliters of bacteria-containing samples were incubated on the biochip electrode for 10 min, the electrode was then rinsed and CV and EIS measurements were taken. The percent change in R_{ct} ratios measured for EPEC WT and ΔespB was calculated and averaged from 20 repeats (five measurements each containing four samples) for each strain. The mean of the averaged ratios and the standard error of the mean were calculated. Differences between the means were statistically significant as indicated by a t-test using an alpha level of 0.05. In order to compare the means of R_{ct} ratios of both strains, standard errors were combined in quadrature.

### Nano Differential Scanning Fluorimetry (NanoDSF)

To assess the thermal stability of mAb-B7, 20 μM mAb-B7 samples were loaded into UV capillaries (NanoTemper Technologies) and analyzed using the nanoDSF Prometheus NT.48. The temperature gradient was set to 1° C./min increase between 15° C. and 95° C. The melting temperatures (Tm) that was derived from protein unfolding was presented by plotting the tryptophan fluorescence at λ=330 nm and λ=350 nm over temperature. The melting temperatures were determined by calculating the maximum of the first derivative and the peak position (at Tₘ) was determined.

### Electrochemical Impedance Spectroscopy (EIS) Measurements

Biosensor measurements were based on Electrochemical Impedance Spectroscopy (EIS) recorded by a commercial potentiostat device (BioLogic, Seyssinet-Pariset, France). EIS was employed to examine the gold electrode before and after modification with the thiolated EspB-specific monoclonal antibody. In a faradaic impedance measurement, a small sinusoidal AC voltage probe is applied (S1 in FIG. 16) while monitoring the current response (S2) at different frequencies. The real (resistive) component of the impedance (determined by the in-phase current response) is plotted against the imaginary (capacitive) component (determined by the out-of-phase current response) with respect to frequency (S3). Both are described by

$${Z}^{′} = {R}_{s} + \frac{{R}_{c t}}{1 + {ω}^{2} {R}_{c t}^{2} {C}_{d l}^{2}} and {Z}^{″} = \frac{{R}_{c t}^{2} {C}_{d l} ω}{1 + {ω}^{2} {R}_{c t}^{2} {C}_{d l}^{2}} ,$$

where Rₛ is the solution resistance, R_{ct} the charge transfer resistance, Cd is the double-layer capacitance, and ω the angular frequency, which is commonly represented in a Nyquist plot. The impedance results of the thus-obtained Nyquist plot are fitted (S4) to an equivalent circuit (Randles circuit) used to interpret the electrochemical system, as shown in the inset of FIG. 20A. An increase in charge transfer resistance (R_{ct}) is attributed to surface adsorption of bound biomolecules (S6).

### The Randles Circuit

To extract the parameter of interest, a generated Nyquist plot is commonly fitted to a model equivalent electronic circuit, Randles circuit. If an analyte affects one of these circuit parameters, then impedance methods can be used for analyte detection. The R_{ct} depicts the opposition experienced to electron movement and it increases in the presence of bound biomolecules. For a one-electron process the R_{ct}, which controls the electron transfer kinetics of Fe(CN)₆^{4-/3-} at the interface of the electrode, can be described by:

$${R}_{c t} = \frac{R T}{{F}^{2} {κ}^{2} C}$$

where R denotes the gas constant, T is temperature, F is Faraday constant, κ⁰ is the electron transfer rate constant and C is the concentration of the electroactive species. The semi-circular region represents a slower charge transfer at higher frequencies whereas the straight line describes a faster mass transfer at lower frequencies. Also, a change in Warburg impedance, Z_{w}, which is dominated by mass transfer can occur when the diffusional transport of electroactive species from the bulk solution to the electrode surface is impeded due to the binding of biomolecules and targets onto the electrode [J. Yeh, B. et al., Sensors Actuators, B Chem. 237 (2016) 329-340]. However, both R_{ct} and Z_{w} depend on the concentration of electroactive species and the applied potential [A. Lasia, Electrochemical Impedance Spectroscopy and its Applications—Andrzej Lasia—Google Books, (2014)https://books.google.com/books?id=1WEgBAAAQBAJ&printsec=frontcover&source=gbs_ge_summary_r&cad=0 #v=onepage&q&f=false].

The Nyquist plots arising from EIS measurements were fitted to the following Randles circuit from which the parameter of interest, R_{ct}, values were calculated.

By using an equivalent circuit, the impedance spectra obtained in our measurements were accurately fitted, as seen by the fitting result of a typical mAb-modified electrode (fitting parameters shown in FIG. 17A).

### Measurement Platform

A PTFE (Teflon™) measurement platform that enables simultaneous interrogation of multiple chips, provides electrical contacts for the chips and connects to a potentiostat device was designed using Solidworks™ software and machined using CNC milling. The resulted platform contains defined slots for 12 chips with liquid chambers of 300 μl, and also enables hydrodynamic measurements. An image of the platform is shown in FIG. 18.

### Example 1

Binding Affinity and Specificity o mAb-EspB-B7

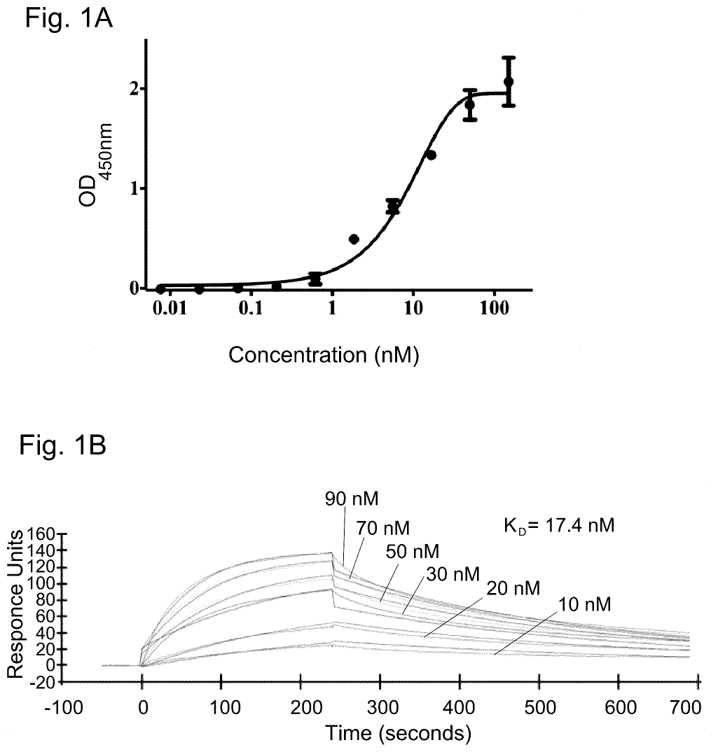

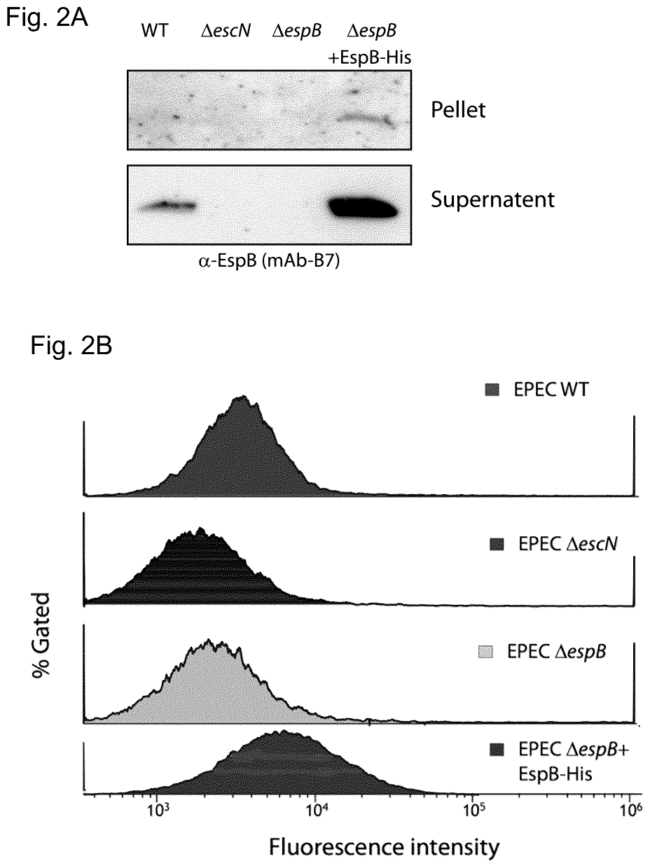

To evaluate the binding affinity of mAb-EspB-B7 to EspB, the inventors performed ELISA (FIG. 1A) and SPR (FIG. 1B) binding assays. It was found that mAb-EspB-B7 binds EspB with high affinity, with a K_{D} value of 17.4 nM. To determine whether mAb-EspB-B7 binds specifically to EspB under native conditions, the binding of mAb-EspB-B7 to WT EPEC, ΔescN (a mutant lacking a functional T3SS), ΔespB (a mutant that does not express and secrete EspB), and ΔespB+EspB-His (an espB null strain that overexpresses plasmid-encoded EspB-His), was examined. The different strains grown under T3SS-inducing conditions were separated into bacterial pellets and supernatants, which were then analyzed by SDS-PAGE and western blot using mAb-EspB-B7 in the detection phase. In agreement with a previous study, a significant secretion of EspB into the bacterial supernatant of WT EPEC was determined, however, not into the supernatants of the ΔescN or ΔespB mutants (FIG. 2A) [8]. Moreover, the inventors observed that overexpression of EspB (ΔespB+EspB-His) resulted in a higher expression of the protein within the bacteria (pellet) as well as a higher secretion into the extracellular medium (FIG. 2A).

### Example 2

### Mab-EspB-B7 Binding to EspB in the Assembled T3SS

To examine whether mAb-EspB-B7 can bind to the native protein in the assembled T3SS, flow cytometry was used. For this purpose, the bacterial strains grown under T3SS-inducing conditions were incubated first with mAb-EspB-B7 and then with a secondary antibody conjugated to a fluorophore. As expected, mAb-EspB-B7 binding was detected in WT EPEC and in the ΔespB strain overexpressing EspB, whereas no or minimal binding was detected in the ΔescN and ΔespB mutant strains (FIG. 2B).

### Example 3

Stability of mAb-EspB-B7 Binding Under Various Physiochemical Conditions

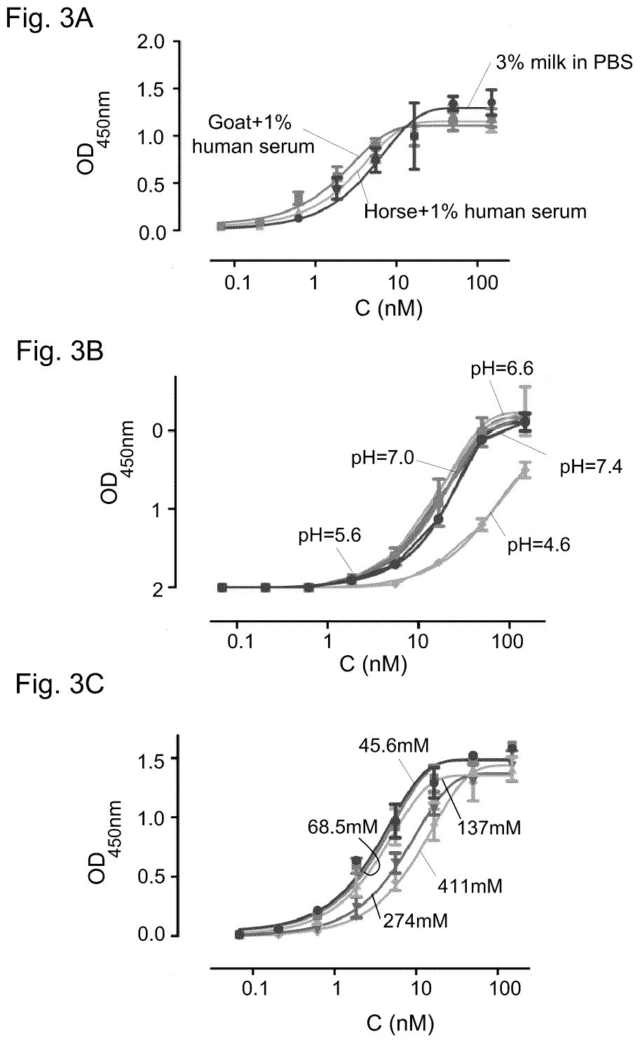

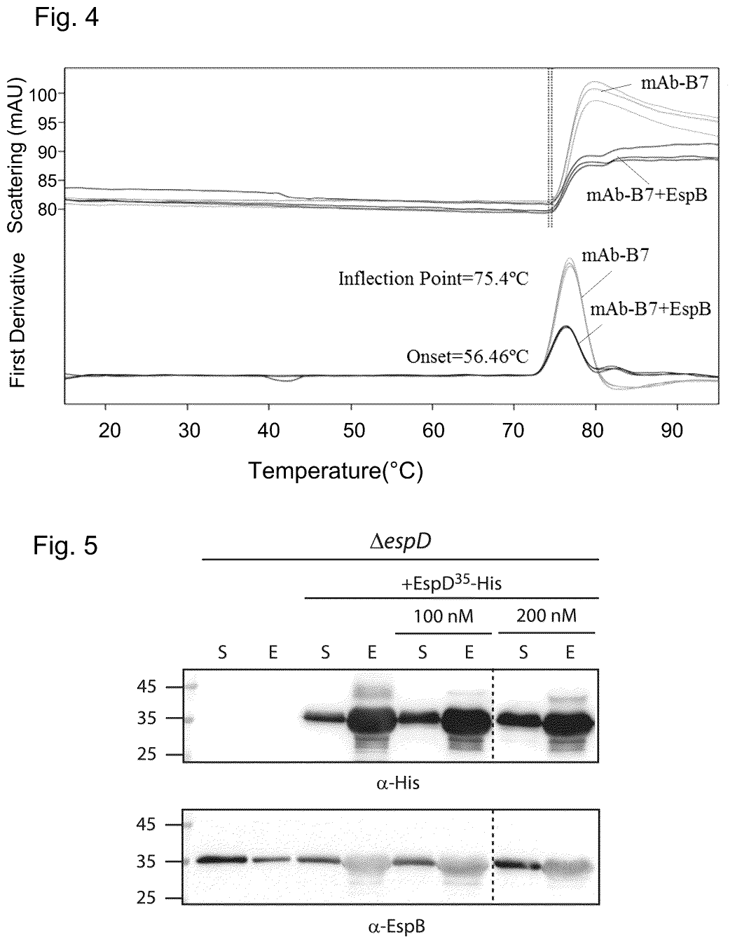

Next, the ability of mAb-EspB-B7 to bind EspB was evaluated under various conditions by ELISA. It was found that incubation of mAb-EspB-B7 in human serum did not compromise the ability of the antibody to bind EspB compared to its binding in a 3% milk solution (FIG. 3A). Examination of mAb-EspB-B7 binding under different pH conditions demonstrated that the binding was essentially not altered under a wide range of pH values (5.6-7.4), with the exception of pH 4.6, at which (as expected) there was a reduction in binding capacity (FIG. 3B). Interestingly, testing mAb-EspB-B7 across a wide range of NaCl concentrations demonstrated that only increased salt concentrations (>250 mM) affected the binding capacity of the antibody (FIG. 3C). Finally, to assess the thermal stability of mAb-EspB-B7, the melting temperature of the antibody was determined, both alone and in complex with purified EspB, by using nanoDSF. The melting temperatures of 75.4° C. and 82° C. for mAb-EspB-B7 alone and in complex with EspB, respectively (FIG. 4), indicated high mAb stability.

### Example 4

Non-Interference of mAb-EspB-B7 with the EspB-EspD Interaction

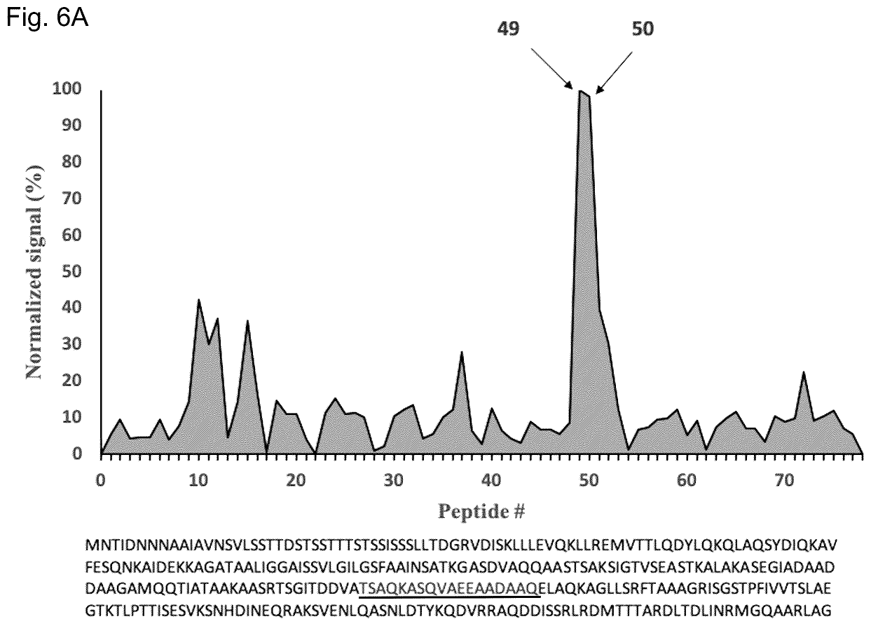

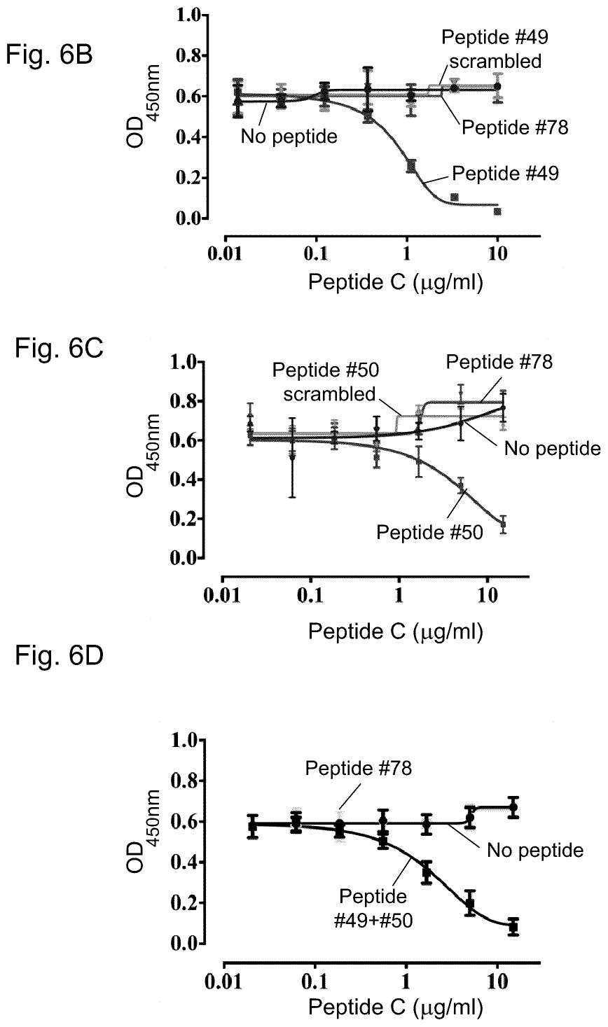

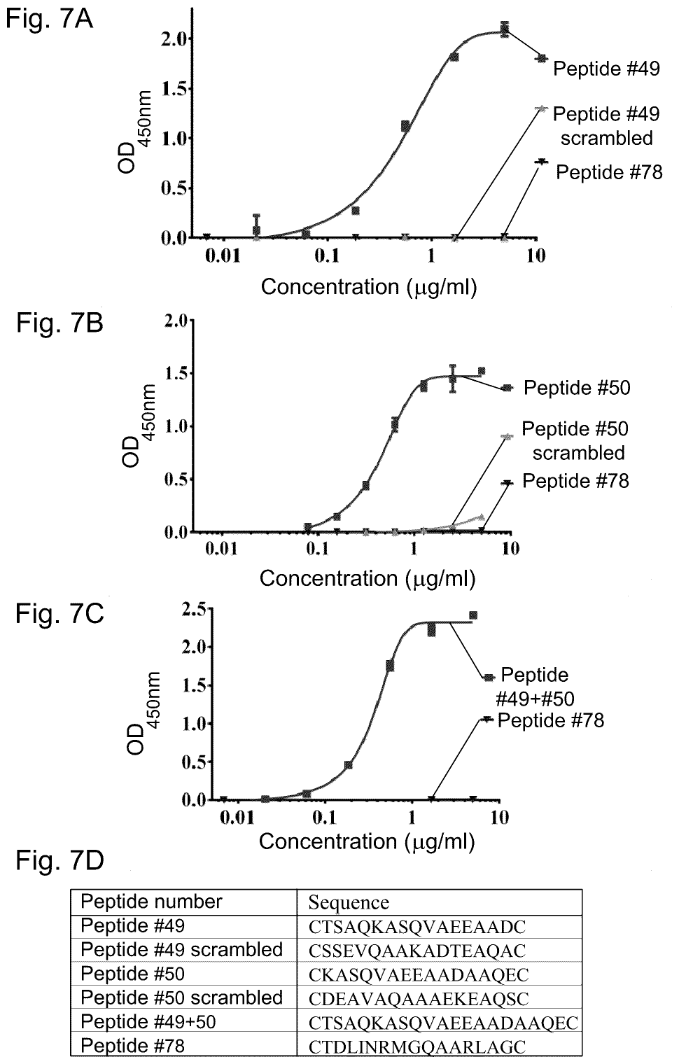

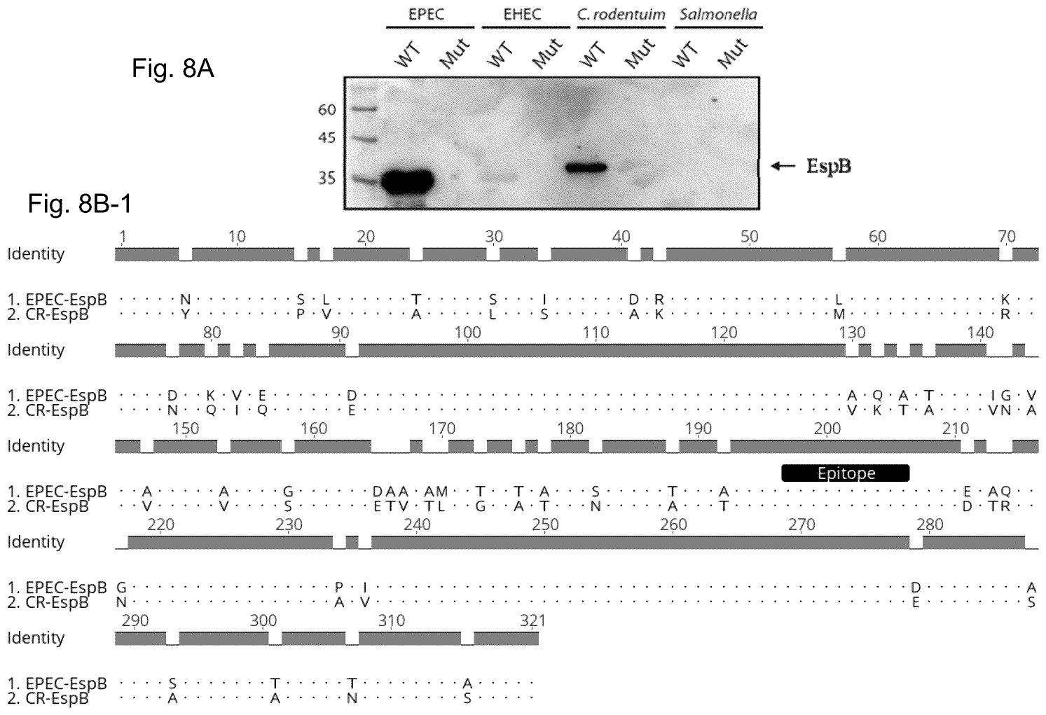

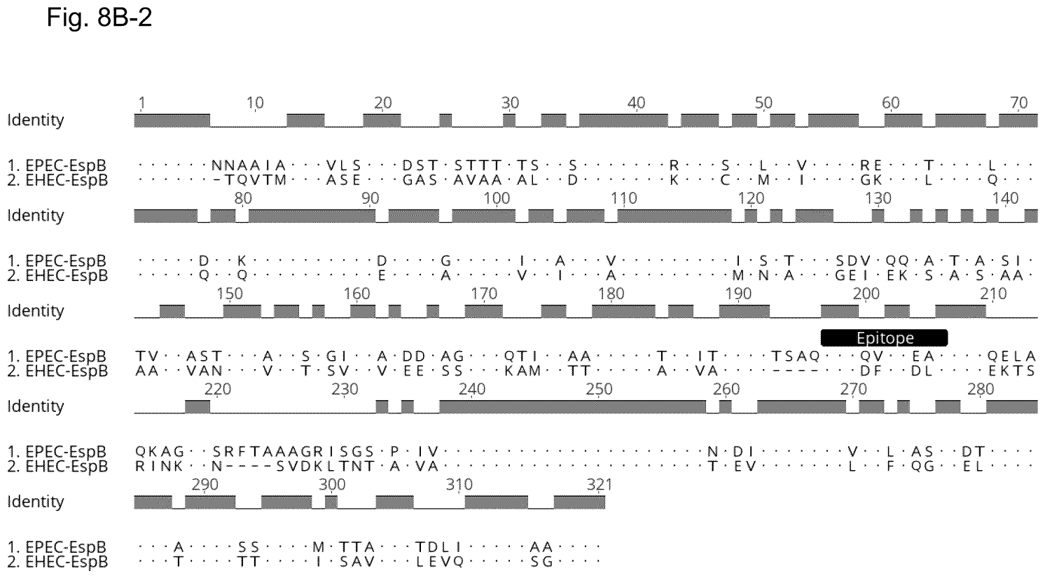

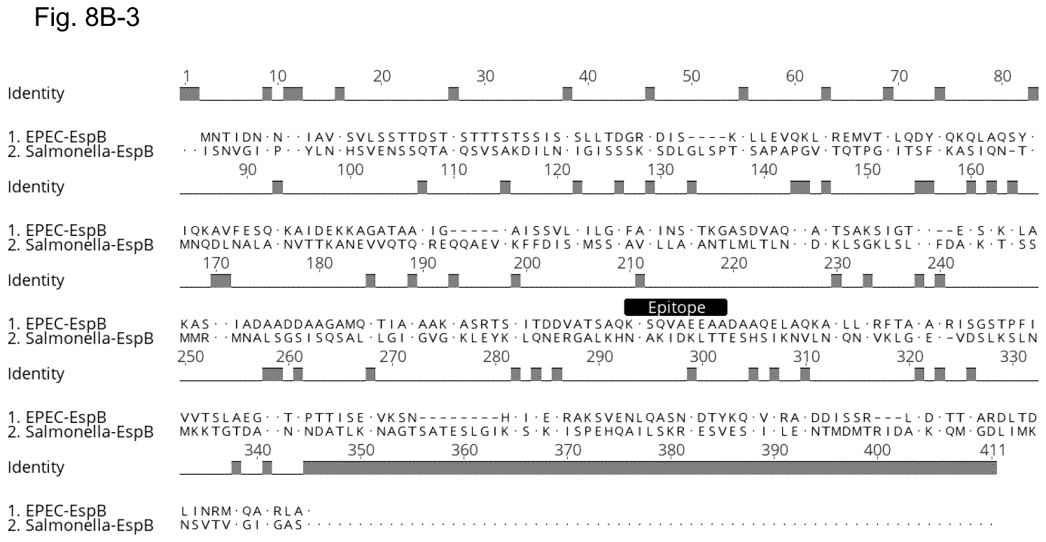

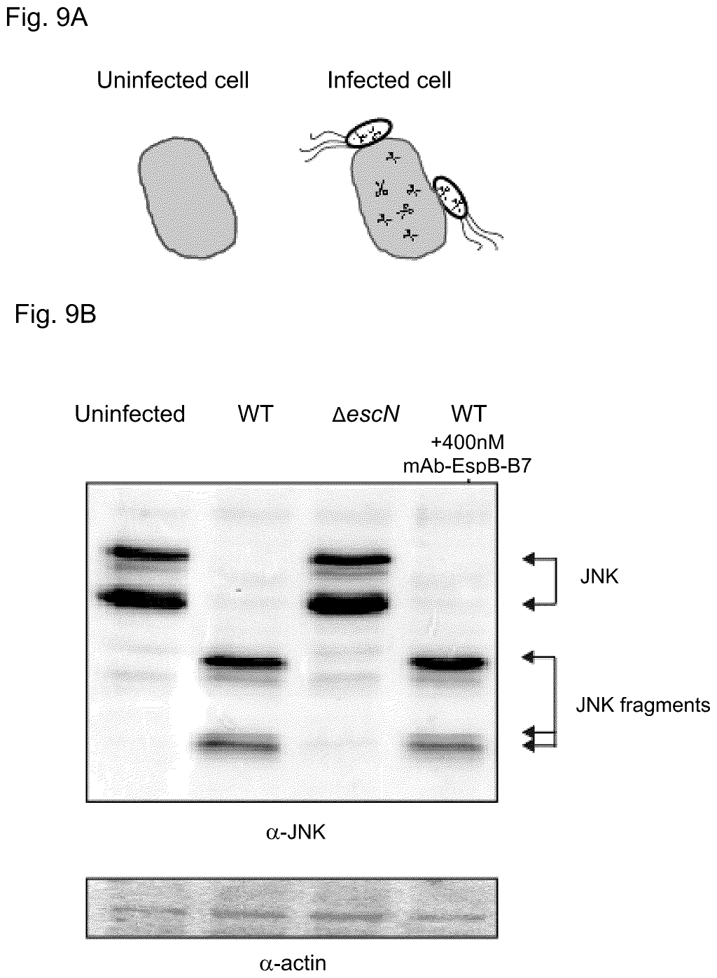

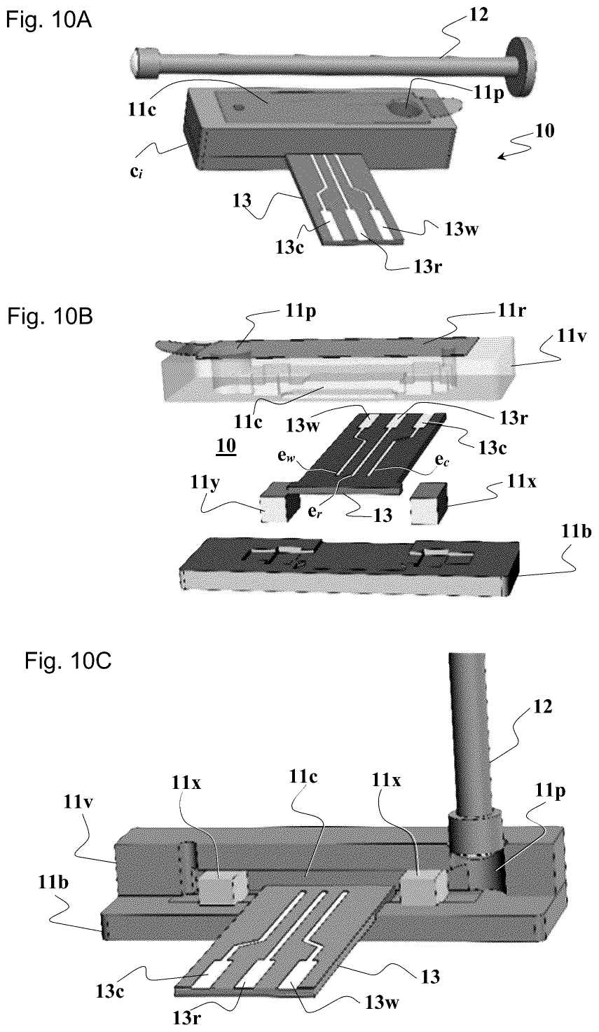

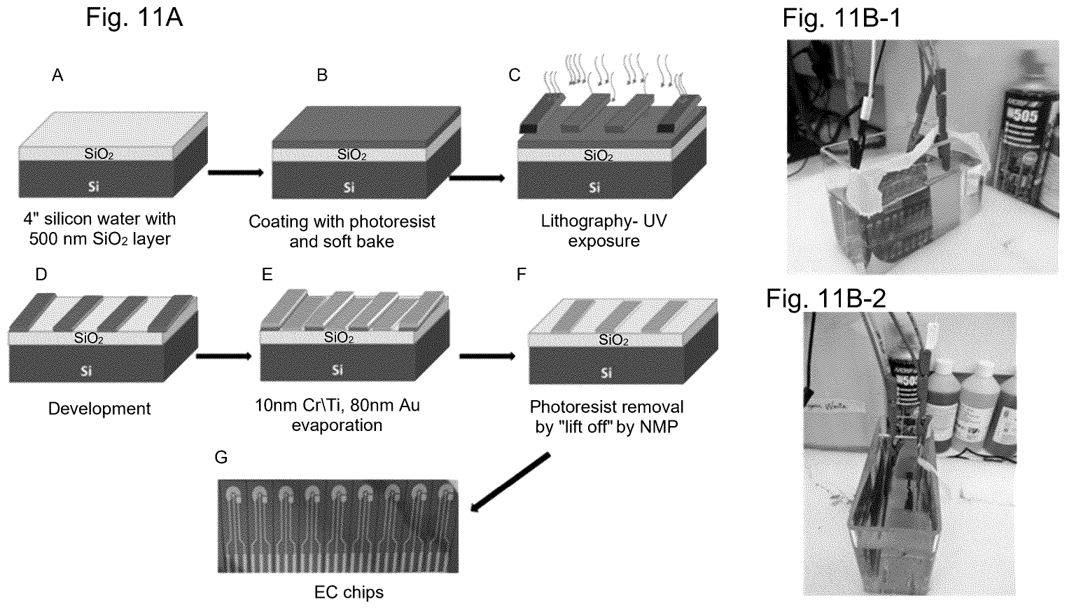

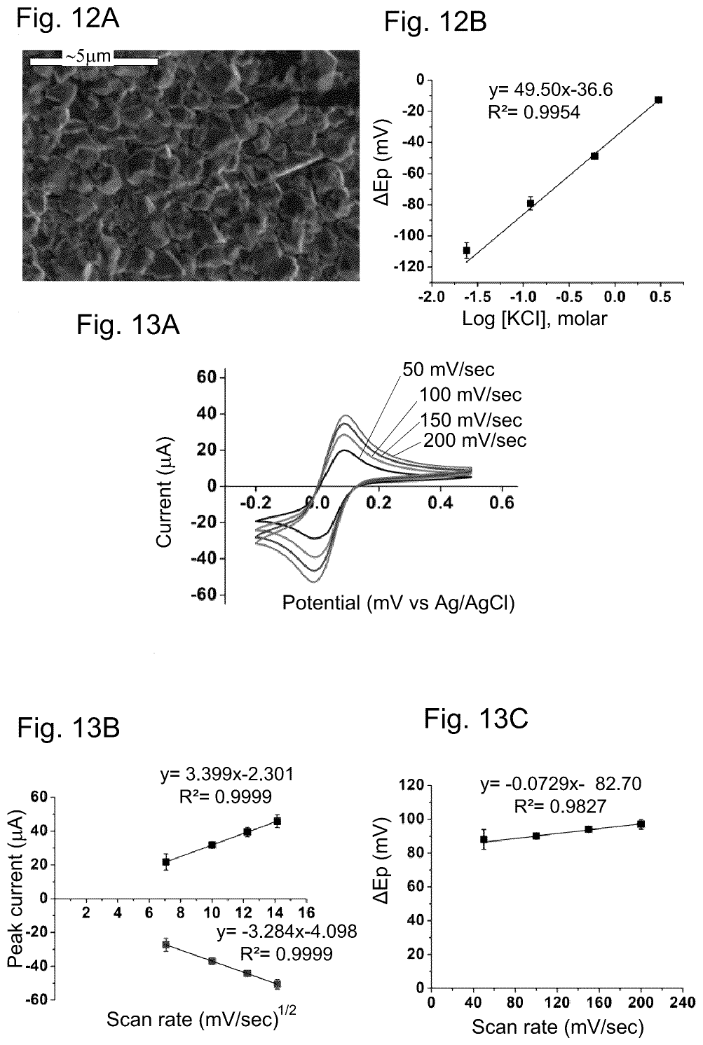

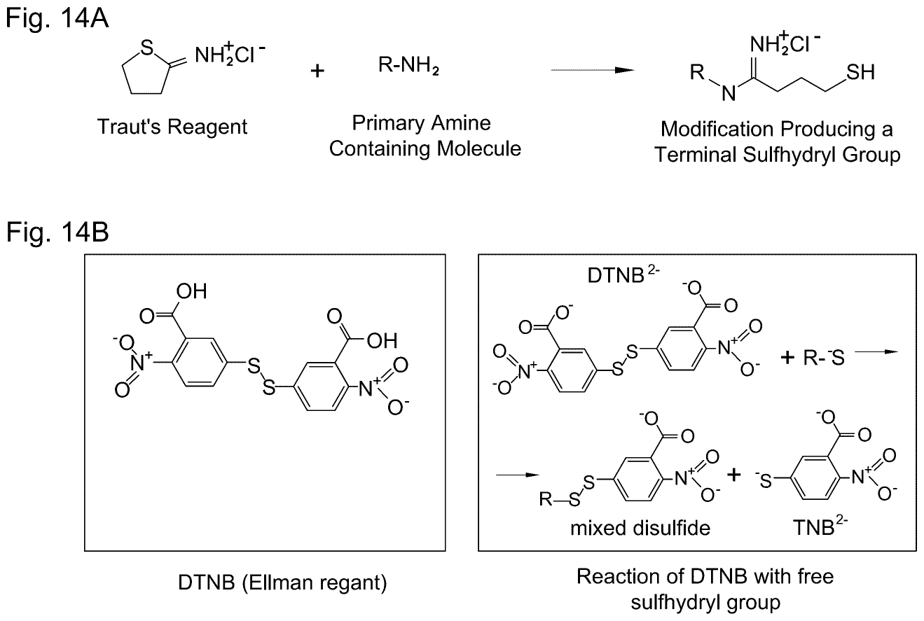

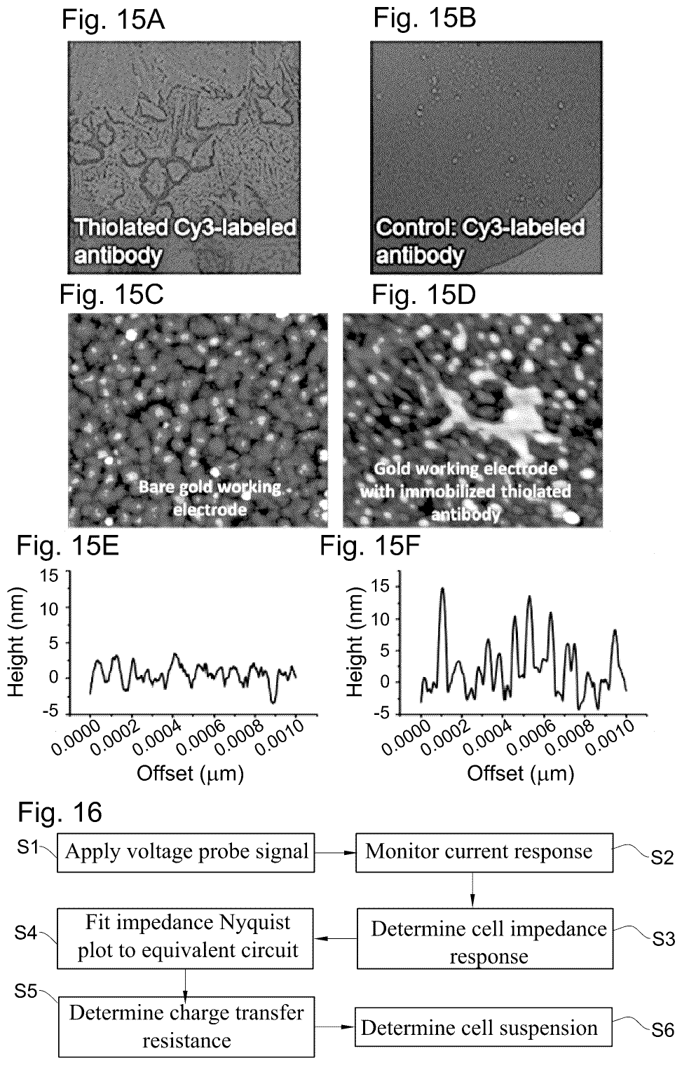

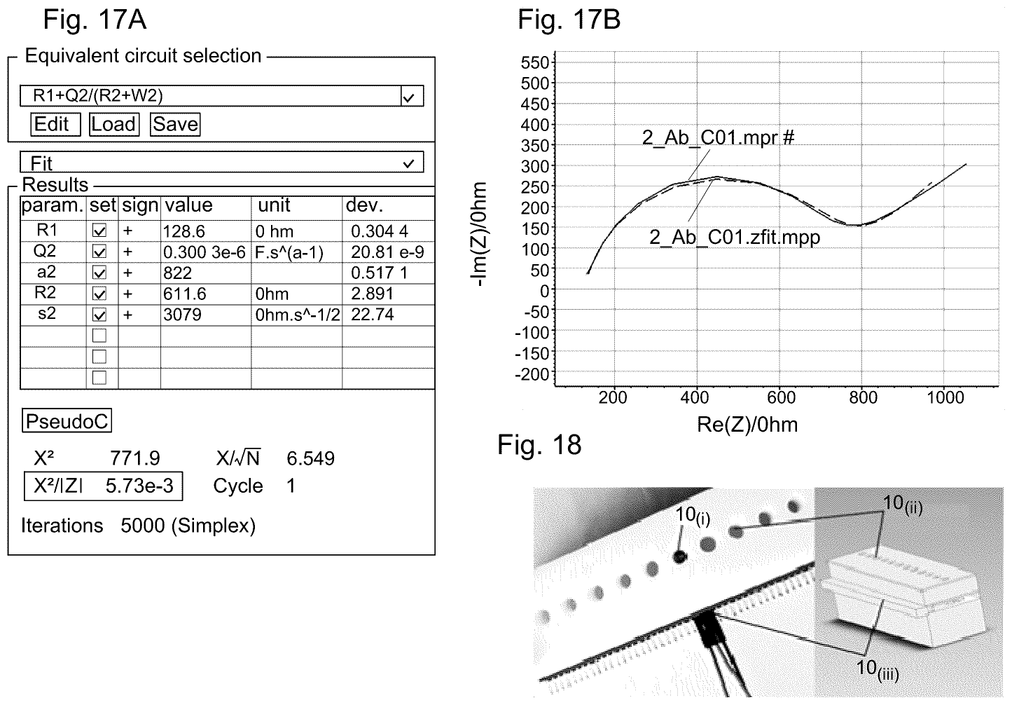

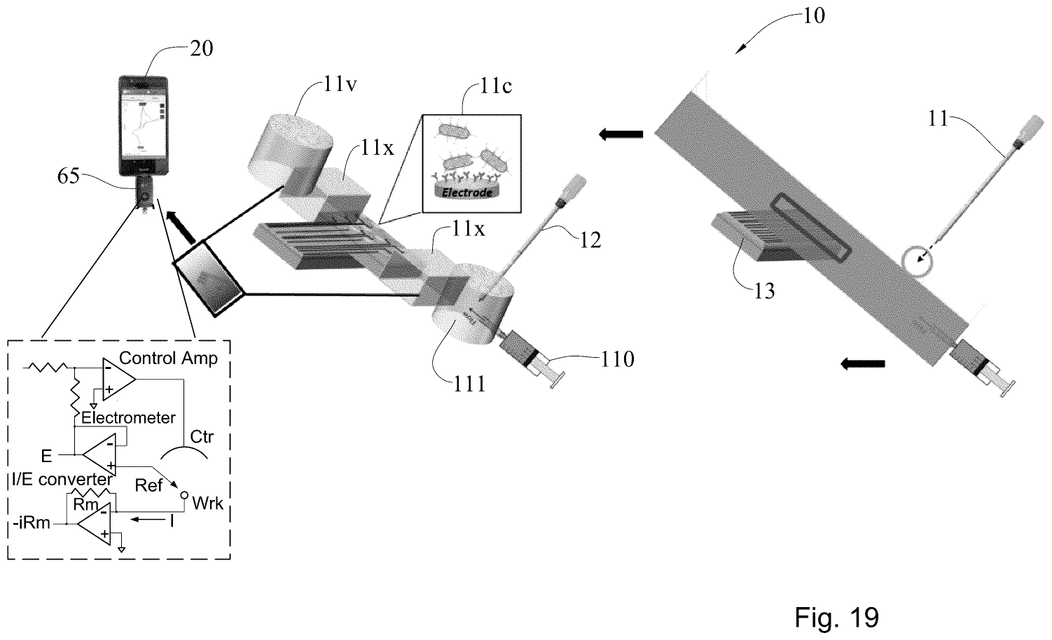

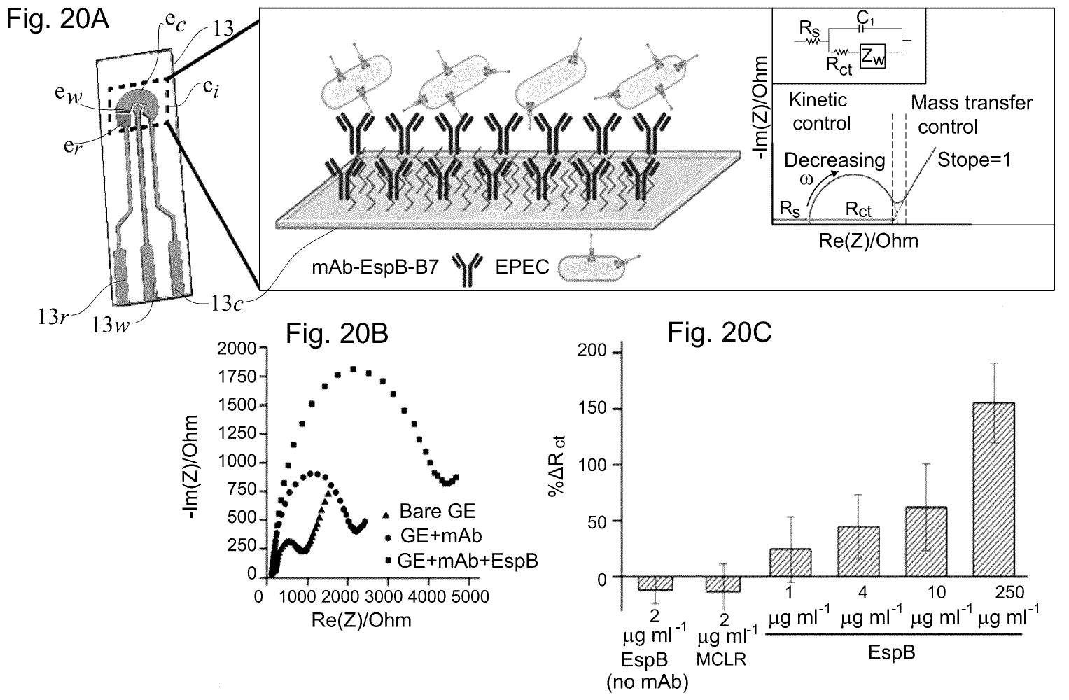

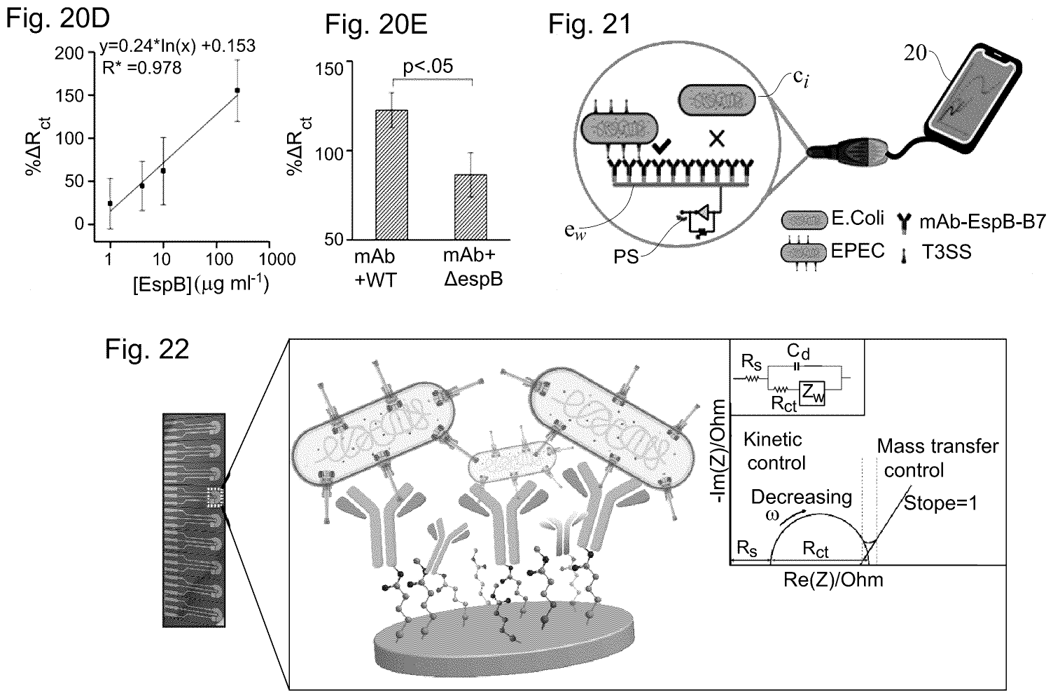

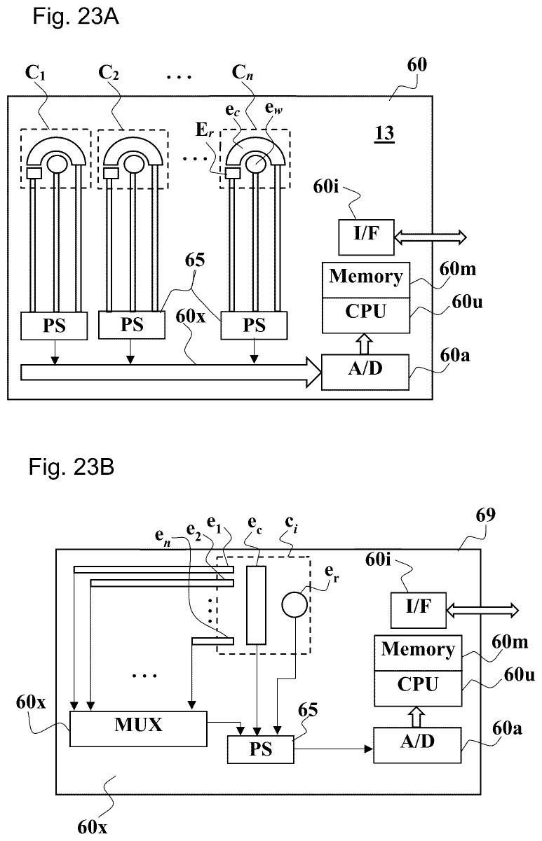

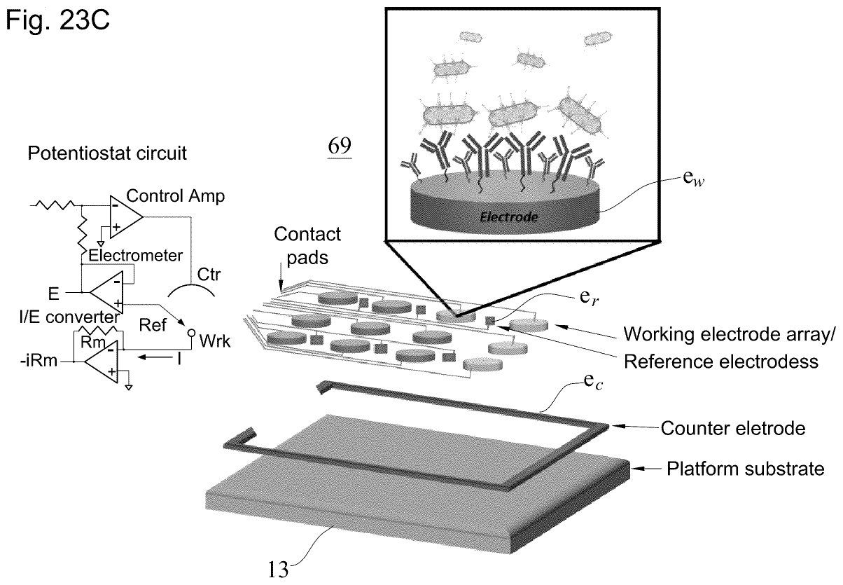

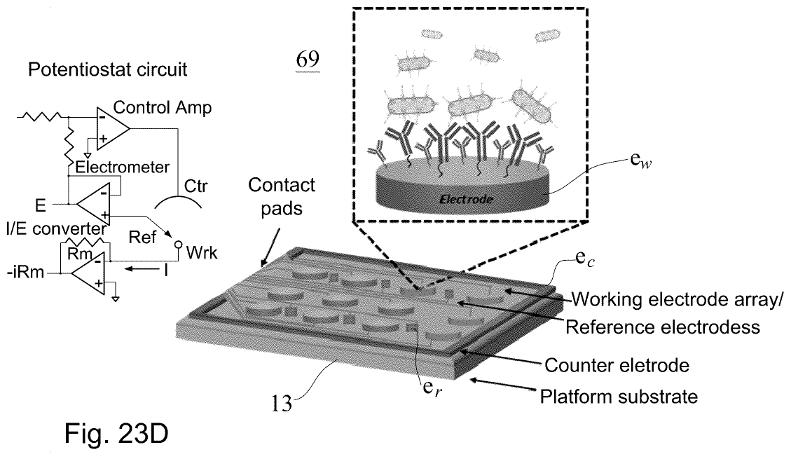

The EspB protein is found in a complex with another T3SS protein, called EspD, within the assembled T3SS. To confirm that the mAb-EspB-B7 epitope is exposed following EspB-EspD interaction, the ability of EspB to co-elute with EspD in the absence or the presence of mAb-EspB-B7 (100 or 200 nM), was evaluated. As observed in FIG. 5, the presence of mAb-EspB-B7 did not affect the co-elution of EspB with EspD, suggesting that mAb-EspB-B7 does not interfere with the EspB-EspD interaction. Low non-specific binding of EspB to the Ni-NTA beads was observed in the negative control (a sample that did not express EspD-³⁵His).

### Example 5

### Mab-EspB-B7 Epitope Mapping

To identify the exact epitope of mAb-EspB-B7 within the EspB protein, a peptide array of 78 cyclic peptides that covers the full sequence of EspB (321 residues long), was designed. Each peptide was 15 residues long, with an overlap of 11 residues between the peptides. Recombinant EspB (full-length) served as a positive control, while BSA served as a negative control. Incubation of mAb-EspB-B7 with the peptide array revealed that mAb-EspB-B7 bound mostly to two cyclic peptides within the array, namely, to peptides #49 (positions 193-207) and #50 (positions 197-211), which have the sequences TSAQKASQVAEEAAD (SEQ ID NO. 33) and KASQVAEEAADAAQE (SEQ ID NO. 35) of the EspB protein, respectively (FIG. 6A). To confirm that this epitope is indeed recognized by mAb-EspB-B7, the following peptides were synthesized: peptide #49; peptide #50; a peptide that comprises the combined sequences of peptides #49 and #50 (TSAQKASQVAEEAADAAQE) (SEQ ID NO. 37); peptide #78 (SEQ ID NO. 38), which was not detected by the mAb-EspB-B7 and was therefore suitable as a negative control; and two peptides with scrambled sequences of peptides #49 and #50. Competitive ELISA between full-length EspB and the cyclic peptides revealed that pre-incubation of peptides #49, #50 or #49+#50 (1 μg/ml) (SEQ ID NOs. 33, 35, 37, respectively), with mAb-EspB-B7 completely abolished the ability of the antibody to bind full-length EspB (FIG. 6B to 6D). To determine whether the competitive effect is derived directly from the binding of mAb-EspB-B7 to the peptides, the ability of mAb-EspB-B7 to recognize and bind these peptides was assessed. As observed in FIG. 7, mAb-EspB-B7 can bind peptides #49, #50 (SEQ ID NOs. 33, 35) and the combined peptide (#49+#50) (SEQ ID NOs. 37), while no binding was detected for the scrambled peptides or for peptide #78 (FIGS. 7A to 7C). These results confirm that the main mAb-EspB-B7 epitope is the KASQVAEEAAD sequence of the EspB protein (peptide sequences are presented in FIG. 7D) (SEQ ID NO. 39).

### Example 6

### Mab-EspB-B7 Specificity Towards EPEC EspB Homologs

To assess the specificity of mAb-EspB-B7 toward EspB homologs in other bacterial pathogens and its potential to be used for detection of bacteria related to other infectious diseases, bacterial cultures grown under T3SS-inducing conditions were centrifuged, and supernatants and pellets were analyzed by SDS-PAGE and western blotting using mAb-EspB-B7. The following WT bacteria and T3SS-mutant strains were cultured: EPEC; enterohemorrhagic E. coli (EHEC), which causes a more severe disease than EPEC in humans; C. rodentium, an EPEC-related mouse pathogen; and Salmonella enterica serovar Typhimurium, which utilizes two T3SSs for virulence. The strongest signal was observed for the WT EPEC supernatant; a significant, but less strong, signal was also detected for C. rodentium, and an even less strong signal, for EHEC (FIG. 8A). However, no signals were detected in the supernatants of Salmonella or any of the T3SS mutant strains. Sequence alignment of EPEC and C. rodentium EspB proteins revealed high conservation between the proteins and full conservation of the mAb-EspB-B7 epitope sequence. Sequence alignment between EPEC and EHEC EspB proteins revealed 80% similarity at the mAb-EspB-B7 epitope region (FIG. 8B).

### Example 7

Effect of mAb-EspB-B7 on Bacterial-Host Cell Interaction

To examine the ability of mAb-EspB-B7 to directly interfere with the bacterial infection of host cells, the translocation activity of WT EPEC was examined in the presence or absence of mAb-EspB-B7. For that purpose, HeLa cells were infected with EPEC strains (WT and ΔescN) and the cleavage pattern of JNK was examined, a host protein that is cleaved by a translocated EPEC effector known as NleD (FIG. 9A). WT EPEC caused extensive degradation of JNK, relative to the uninfected sample and the samples infected with the ΔescN mutant strain (FIG. 9B). The inventors found that, HeLa cells infected by WT EPEC in the presence of a high concentration of mAb-EspB-B7 (400 nM) showed translocation activity similar to that of WT EPEC infection with no addition of antibody. These results suggest that while mAb-EspB-B7 was capable of binding EspB with high affinity and specificity, this binding did not interfere with the protein function and did not inhibit the T3SS translocation activity in an ex vivo model.

### Example 8

Development of mAb-EspB-B7-Based Biosensor and its Application in Detecting Purified EspB Protein and EspB-Presenting Bacteria

The potential of the developed mAb-EspB-B7 in diagnostic applications may be exploited by integrating it with an electrochemical biosensor. In particular, impedimetric immunosensors show great promise in rapidly detecting low concentrations of target antigens within a highly simplified testing setup. A general scheme of the disclosed device is shown in FIG. 19. FIG. 19 shows an electrochemical chip device providing an electrode arrangement within contact with a sample. The sample is provided by a sample collector and may be pushed into a measurement chamber using a syringe/plunger. The inventors sought to demonstrate this potential by constructing a miniature electrochemical biochip functionalized with mAb-EspB-B7, as illustrated in FIG. 20A working electrode of the electrode arrangement is pre-treated by biofunctionalization with mAb-EspB-B7. Impedance measurement between the electrodes provides data indicative of agents bound to the binding site. The impedance measurements may be represented by Nyquist plots that were fitted to an equivalent model circuit from which charge transfer resistance (R_{ct}) values were obtained. The EIS was recorded for the electrodes before and after antibody immobilization and these were compared with measurements taken after a 10 min incubation of purified EspB protein at varying concentrations. Using the Nyquist plot (FIG. 20B) the effect of mAb-EspB-B7 immobilization on the R_{ct} was observed. In a bare electrode, the resistance to charge transfer is small and impedance is dominated by mass transfer (diffusion of the electroactive species), the so-called Warburg impedance, which is evident in low frequencies [17]. The Warburg impedance is considerably decreased following mAb immobilization as mass transfer is no longer a significant factor. Instead, the contribution of R_{ct} to the impedance is now largely dominant, as an insulating layer of biomolecules is attached to the surface. Following a brief incubation of 250 μg/mL purified EspB solution, a significant increase in R_{ct} was observed, which is directly correlated with the bound antigen concentration further adding to the resistive component of the impedance. It was found that the addition of purified EspB protein affects the R_{ct} in a dose-dependent manner, enabling the distinction between different concentrations (FIG. 20C). In addition, incubation of a non-specific antigen (microcystin toxin) showed no effect on the measured R_{ct}. Similarly, no effect was observed from purified EspB protein directly incubated on a bare electrode (FIG. 10C), indicating that the change in R, reflects the specific binding of EspB to the mAB-EspB-B7 antibody. The relative change in R_{ct} exhibits an exponential dependence on concentration, as seen in FIG. 20D.

To examine the applicability of mAb-EspB-B7-based electrochemical bio-sensing in detecting whole bacteria harboring T3SS (and present EspB), WT EPEC and ΔespB mutant strains were grew under T3SS-inducing conditions and incubated bacterial samples on the biochip. As shown in FIG. 20E, higher R_{ct} values were consistently recorded for EPEC WT (mean R_{ct} change=1.22±0.09) compared to the ΔespB mutant strain (mean R_{ct} change=0.86±0.12). The minimal binding of the ΔespB strain was likely due to nonspecific adsorption. It should be noted that following centrifugation and lack of T3SS-inducing conditions, shedding of the complex is likely to occur, consistent with the western blot analysis shown in FIG. 2A. Nevertheless, the obtained signals were shown to be significantly higher (36%±15) in response to EPEC WT compared to the ΔespB strain (p=0.03). Overall, these results demonstrated the potential of the biosensor of the present disclosure, to detect EspB, both in its secreted form as well as an integral component of the assembled T3SS complex in the context of whole bacteria.

1-48. (canceled)

49. A biosensor chip device usable for identifying and/or quantifying a target in a sample by electrochemical impedance spectroscopy (EIS) analysis, the chip device comprising:
a plurality of electrodes connectable to at least one electronic device;
wherein at least one of said electrodes is a working electrode, said working electrode is connected directly or indirectly to at least one target binding site and/or moiety, wherein said target binding site and/or moiety specifically binds said at least one target or any component thereof, and wherein said plurality of electrodes is configured for electrochemical impedance spectroscopy (EIS) analysis of said sample.

50. The biosensor chip device of claim 49, wherein at least one of:
(a) the biosensor chip device further comprises a packaging assembly configured to sealably enclose said electrodes portion of the substrate and define said measurement chamber encompassing said electrodes;
(b) the plurality of electrodes comprising at least one working electrode, at least one counter electrode configured to introduce electrical currents into said measurement chamber, and at least one reference electrode for measuring electrical voltage between said at least one working electrode and said at least one reference electrode, wherein said at least one working electrode is connected directly or indirectly to at least one said target binding site and/or moiety;
(c) the biosensor chip device further comprises at least one inlet for introducing said sample into said measurement chamber; and at least one inlet filter for selectively passing said sample from said inlet into said measurement chamber, optionally, comprising an outlet formed in the packaging assembly and at least one outlet filter for selectively passing sample material from the measurement chamber to said outlet; and
(d) wherein the packaging assembly comprises a base portion configured to receive the electrodes portion of the substrate, and a cover portion having an open cavity and configured to sealably attach to said base portion over said electrodes portion of the substrate and define the measurement chamber by its open cavity.

51. The chip device of claim 49, wherein at least one of:
(a) said at least one electronic device comprises a plurality of potentiostat circuitries, said device comprising a plurality of measurement chambers, each comprising at least three of the plurality of electrodes defining a working electrode, a reference electrode, and a counter electrode, and a respective plurality of potentiostat circuitries each of which are electrically connected to the at least three electrodes of its respective measurement chamber; and
(b) the plurality of electrodes comprises a plurality of working electrodes, at least one reference electrode, and at least one counter electrode, and wherein the device comprises a potentiostat circuitry and a multiplexer device configured to selective transfer signals measured by said plurality of working electrodes to said potentiostat circuitry.

52. The chip device of claim 49, wherein at least one of:
(a) said target is at least one pathogen expressing at least one component of the Type III Secretion System (T3SS);
(b) said at least one target binding site and/or moiety is comprised within at least one antibody that recognizes and binds at least one component of the T3SS, or any combination or complex thereof, said antibody or any functional fragments thereof is immobilized to said at least one working electrodes;
(c) said component of said T3SS is at least one of the Enteropathogenic Escherichia coli (EPEC) secreted protein A (EspA), EPEC secreted protein B (EspB), and EPEC secreted protein D (EspD), or any fragments or peptides thereof, and any combination or complex thereof; and
(d) wherein said at least one antibody recognizes and binds the EspB protein, or any complex thereof with EspD protein.

53. The chip device of claim 52, wherein said at least one antibody recognizes and binds the EspB protein, said antibody comprises a heavy chain complementarity determining region (CDRH) 1 comprising the amino acid sequence GFTFSHYA, as denoted by SEQ ID NO. 6, CDRH2 comprising the amino acid sequence INSNGDST, as denoted by SEQ ID NO. 10, CDRH3 comprising the amino acid sequence ARDRRAGYFDYW, as denoted by SEQ ID NO. 14, and a light chain complementarity determining region (CDRL) 1 comprising the amino acid sequence RDNIGKNY as denoted by SEQ ID NO. 22, a CDRL2 comprising the amino acid sequence RNN as denoted by SEQ ID NO. 26, and a CDRL3 comprising the amino acid sequence SAWDTSLNA as denoted by SEQ ID NO. 30, or any derivative, variant and biosimilar thereof.

54. The chip device of claim 49, wherein at least one of:
(a) said pathogen is a bacterial pathogen, said bacteria is at least one Multiple Drug Resistant (MDR) bacteria, optionally, said MDR bacteria is at least one of Enteropathogenic Escherichia coli (EPEC) and Enterohemorrhagic Escherichia coli (EHEC); and
(b) said sample is a biological sample or an environmental sample.

55. A kit comprising:
(a) at least one biosensor chip device according to claim 49, usable for identifying and/or quantifying a target in a sample by electrochemical impedance spectroscopy (EIS) analysis, the chip device comprising:
a plurality of electrodes connectable to at least one electronic device;
wherein at least one of said electrodes is a working electrode, said working electrode is connected directly or indirectly to at least one target binding site and/or moiety, wherein said target binding site and/or moiety specifically binds said at least one target or any component thereof, and wherein said plurality of electrodes is configured for electrochemical impedance spectroscopy (EIS) analysis of said sample;
optionally, said kit comprises at least one of:
(b) at least one control sample and/or control standard value; and
(c) instructions for use.

56. A method for identifying and/or quantifying at least one target in a sample, the method comprising:
contacting a plurality of electrodes comprising at least one working electrode, at least one reference electrode, and at least one counter electrode, with said sample, wherein said at least one working electrode is connected directly or indirectly to at least one target binding site and/or moiety;
applying voltage signal between said at said least one working electrode and said at least one reference electrode, and determining electrical current through said at least one working electrode in response to said voltage signals for a selected number of one or more signal frequencies;
determining relations between electrical current response and voltage signal for said one or more signal frequencies; and determining electrical impedance between the at least one working electrode and the at least one counter electrode;
wherein impedance variation being indicative of presence and/or quantity of said at least one target in said sample.

57. The method of claim 56, further comprising processing electrical impedance determined based on one or more voltage signal frequencies for determining charge transfer electrical resistance between the at least one working electrode and the at least one reference electrode, and determining presence of said at least one target in said sample whenever said charge transfer electrical resistance is greater than a predetermined threshold value, optionally, wherein determining the charge transfer electrical resistance comprises determining an electrical circuit model representing charge transfer between the electrodes, said electrical circuit may comprise capacitance model connected in parallel to inductance model and charge transfer electrical resistance model, thereby allowing to determine charge transfer electrical resistance in accordance with total impedance of the circuit.

58. The method of claim 56, wherein at least one of:
(a) said target is at least one pathogen expressing at least one component of the Type III Secretion System (T3SS);
(b) said at least one target binding site and/or moiety is comprised within at least one antibody that recognizes and binds at least one component of the T3SS, or any combination or complex thereof, said antibody or any functional fragments thereof is immobilized to at least one of said working electrodes;
(c) said component of said T3SS is at least one of the Enteropathogenic Escherichia coli (EPEC) secreted protein A (EspA), EPEC secreted protein B (EspB), and EPEC secreted protein D (EspD), and any combination or complex thereof;
(d) said at least one antibody recognizes and binds the EspB protein, or any complex thereof with EspD protein.

59. The method of claim 58, wherein said at least one antibody recognizes and binds the EspB protein, said antibody comprises a heavy chain complementarity determining region (CDRH) 1 comprising the amino acid sequence GFTFSHYA, as denoted by SEQ ID NO. 6, CDRH2 comprising the amino acid sequence INSNGDST, as denoted by SEQ ID NO. 10, CDRH3 comprising the amino acid sequence ARDRRAGYFDYW, as denoted by SEQ ID NO. 14, and a light chain complementarity determining region (CDRL) 1 comprising the amino acid sequence RDNIGKNY as denoted by SEQ ID NO. 22, a CDRL2 comprising the amino acid sequence RNN as denoted by SEQ ID NO. 26, and a CDRL3 comprising the amino acid sequence SAWDTSLNA as denoted by SEQ ID NO. 30, or any derivative, variant and biosimilar thereof.

60. The method of claim 56, wherein at least one of:
(a) said pathogen is a bacterial pathogen, said bacteria is at least one Multiple Drug Resistant (MDR) bacteria;
(b) said MDR bacteria is at least one of Enteropathogenic Escherichia coli (EPEC) and Enterohemorrhagic Escherichia coli (EHEC);
(c) said sample is biological sample or an environmental sample.

61. The method of claim 56, for the diagnosis of an infectious condition caused by or associated with at least one T3SS expressing pathogen, in a subject.

62. A method for identifying and/or quantifying at least one target in a sample, the method comprising:
contacting said sample with at least one working electrode, at least one reference electrode, and at least one counter electrode, or any biosensor chip or kit comprising said electrodes, wherein said at least one working electrode is connected directly or indirectly to at least one target binding site and/or moiety;
measuring electrical voltages between said at least one working electrode and said at least one reference electrode in response to electric currents of different frequencies applied between said at least one working electrode and said at least one counter electrode;
determining electrical impedances based on the measured electrical voltage and the electric currents applied at the different frequencies;
determining a charge transfer electrical resistance based on the determined impedances; and
determining presence of said at least one target in said sample whenever said charge transfer electrical resistance is greater than a predetermined threshold value.

63. The method of claim 62, wherein at least one of:
(a) the determining a charge transfer electrical resistance comprises determining an electrical circuit model equivalent to a circuitry defined by the electrodes and the sample based on the determined electrical impedances, optionally, the determining of the equivalent electrical circuit model comprises correlating Nyquist presentation of the electrical impedances determined at the different frequencies to Nyquist presentation of electrical impedances of the equivalent electrical circuit model;
(b) the measurement chamber comprises a plurality of working electrodes, each connected directly or indirectly to at least one target binding site and/or moiety, and wherein the method comprising determining a respective plurality of electrical impedances associated with at least some of said plurality of working electrodes, and determining the charge transfer electrical resistance based of the determined respective plurality of electrical impedances; and
(c) the measurement chamber comprises a plurality of working electrodes, each connected directly or indirectly to at least one target binding site and/or moiety, and a respective plurality of reference electrodes, and wherein the method comprising determining a respective plurality of electrical impedances associated pairs of said working and reference electrodes, and determining the charge transfer electrical resistance based of the determined respective plurality of electrical impedances.

64. The method of claim 62, wherein at least one of:
(a) said target is at least one pathogen expressing at least one component of the Type III Secretion System (T3SS);
(b) said at least one target binding site and/or moiety is comprised within at least one antibody that recognizes and binds at least one component of the T3SS, or any combination or complex thereof, said antibody or any functional fragments thereof is immobilized to at least one of said working electrodes;
(c) said component of said T3SS is at least one of the Enteropathogenic Escherichia coli (EPEC) secreted protein A (EspA), EPEC secreted protein B (EspB), and EPEC secreted protein D (EspD), and any combination or complex thereof; and
(d) said at least one antibody recognizes and binds the EspB protein, or any complex thereof with EspD protein.

65. The method of claim 64, wherein said at least one antibody recognizes and binds the EspB protein, said antibody comprises a heavy chain complementarity determining region (CDRH) 1 comprising the amino acid sequence GFTFSHYA, as denoted by SEQ ID NO. 6, CDRH2 comprising the amino acid sequence INSNGDST, as denoted by SEQ ID NO. 10, CDRH3 comprising the amino acid sequence ARDRRAGYFDYW, as denoted by SEQ ID NO. 14, and a light chain complementarity determining region (CDRL) 1 comprising the amino acid sequence RDNIGKNY as denoted by SEQ ID NO. 22, a CDRL2 comprising the amino acid sequence RNN as denoted by SEQ ID NO. 26, and a CDRL3 comprising the amino acid sequence SAWDTSLNA as denoted by SEQ ID NO. 30, or any derivative, variant and biosimilar thereof.

66. The method of claim 62, wherein at least one of:
(a) said pathogen is a bacterial pathogen, said bacteria is at least one Multiple Drug Resistant (MDR) bacteria, optionally, said MDR bacteria is at least one of Enteropathogenic Escherichia coli (EPEC) and Enterohemorrhagic Escherichia coli (EHEC);
(b) said sample is biological sample or an environmental sample; and
(c). the method is for the diagnosis of an infectious condition caused by or associated with at least one T3SS expressing pathogen, in a subject.

67. A method of treating, preventing, ameliorating, reducing or delaying the onset of an infection by at least one bacteria expressing at least one T3SS in a subject in need thereof, the method comprising:
(a) classifying a subject as infected by said bacteria if the presence of at least one T3SS component is determined in at least one sample of said subject, wherein determination of the presence of said at least one T3SS component in said sample is performed by the method according to claim 62, the method comprising steps of:
contacting said at least one sample of said subject with a plurality of electrodes comprising at least one working electrode and at least one reference electrode, or any biosensor chip or kit comprising said electrodes, wherein said at least one working electrode is connected directly or indirectly to at least one target binding site and/or moiety;
measuring electrical voltages between said at least one working electrode and said at least one reference electrode in response to electric currents of different frequencies applied by said at least one counter electrode;
determining electrical impedances based on the measured electrical voltage and the electric currents applied at the different frequencies;
determining a charge transfer electrical resistance based on the determined impedances; and
determining presence of said bacteria expressing at least one T3SS in said sample whenever said charge transfer electrical resistance is greater than a predetermined threshold value, thereby classifying said subject as infected by said bacteria;
(b) administering to a subject classified as an infected subject in step (a), a therapeutically effective amount of at least one anti-bacterial agent.

68. At least one system comprising at least one biosensor chip device according to claim 49, usable for identifying and/or quantifying a target in a sample by electrochemical impedance spectroscopy (EIS) analysis, the chip device comprising:
a plurality of electrodes connectable to at least one electronic device;
wherein at least one of said electrodes is a working electrode, said working electrode is connected directly or indirectly to at least one target binding site and/or moiety, wherein said target binding site and/or moiety specifically binds said at least one target or any component thereof, and wherein said plurality of electrodes is configured for electrochemical impedance spectroscopy (EIS) analysis of said sample.
